SEQUENCE LISTING (2 sequences)

SEQ ID NO: 1 - DNA, 21 bp, synthetic construct
  source 1..21: mol_type = other DNA
atacctcatggtaatcctgtt

SEQ ID NO: 2 - RNA, 23 nt, synthetic construct
  source 1..23: mol_type = other RNA
tggactccgataacggagtatgt

(19) United States
(12) Patent Application Publication
(10) Pub. No.: US 2025/0049031 A1
(43) Pub. Date: Feb. 13, 2025

(54) METHOD FOR PATHOGENS, MICROORGANISMS, AND PARASITES INACTIVATION

(71) Applicant: New York Blood Center, Inc., New York, NY (US)

(72) Inventors: David R. Tabatadze, Worcester, MA (US); Ivan B. Yanachkov, Shrewsbury, MA (US); Boris V. Zavizion, Brookline, MA (US); Bruce S. Sachais, Wilmington, DE (US)

(21) Appl. No.: 18/659,817

(22) Filed: May 9, 2024

Related U.S. Application Data
(63) Continuation of application No. 17/262,790, filed on Jan. 25, 2021, now abandoned.
(60) Provisional application No. 62/711,241, filed on Jul. 27, 2018.

(51) Int. Cl. A01N 43/44; A01N 25/10; A01N 25/32

(52) U.S. Cl. CPC A01N 43/44; A01N 25/10; A01N 25/32

(57) ABSTRACT

The invention provides a method for inactivation or reduction of pathogens, microorganisms or parasites in a sample, media, composition, utility, device, surface or organism by treatment with an alkylating compound of Structure I, followed by elimination or reduction of the residual compound with Structure I by treatment with a neutralizing agent, which eliminates or reduces the toxicity or other undesirable properties of the alkylating compound with Structure I. The neutralizing agent may be present in a treatment solution or be part of a solid-phase agent, and preferably acts by eliminating the alkylating properties of the compound of Structure I.

### CROSS REFERENCE TO RELATED APPLICATIONS

The present application is a continuation of U.S. patent application Ser. No. 17/262,790, filed Jan. 25, 2021, which is a 371 of international application serial no. PCT/US2019/043675, fled Jul. 26, 2019, which claims the benefit of U.S. provisional patent application Ser. No. 62/711,241, fled Jul. 27, 2018, the entire contents each of which are incorporated herein by reference.

### SEQUENCE LISTING

This application contains a Sequence Listing that has been submitted electronically as an XML file named “58434-0004002 SL_ST26.” The XML file, created on Oct. 10, 2024, is 2,830 bytes in size. The material in the XML file is hereby incorporated by reference in its entirety.

### FIELD OF THE INVENTION

The present invention relates to compositions and methods for use in the inactivation or reduction of pathogens, microorganisms or parasites in medicine, biologics, medical devices, and cosmetics, in industry and in research. More particularly, the invention provides compositions and methods for the inactivation and/or reduction of pathogens, microorganisms or parasites (e.g. contaminants) in a sample, media, composition, utility, device, surface or organism by treatment with an alkylating compound, followed by the elimination or reduction of the residual alkylating compound and/or its by-products.

### BACKGROUND

Existing pathogens and infectious disease organisms, as well as new and emerging ones, and other undesired organisms (e.g. contaminant) in general, including structures such as biofilms or biofouling create significant problems in a wide range of fields, including medicine, manufacturing, production of pharmaceuticals, biologics, cosmetics, food, medical devices, research, and in other industries. It is therefore important to inactivate pathogens or undesired organisms in a broad range of samples, including organisms, or products and compositions, including food, drugs, plants, blood or blood products, bodily fluids, medium originated from eukaryotes or prokaryotes, vaccines or vaccine preparation compositions, cosmetics, biologics and pharmaceutical compositions, or in or on the surface of utensils, devices, or utilities of household, industrial or medical use, including fluid conduits, heat exchangers or aquatic vessels.

Currently, there is no universal pathogen, undesired microorganism, or parasite reduction technique that are broadly applicable for inactivating organisms in samples and compositions or utilities. Some amphiphilic quaternary ammonium salts are quite universal disinfectants, especially at higher concentration, yet they are inactive against non-enveloped viruses. Small, reactive molecules, such as chlorine gas, sodium hypochlorite, ethylene oxide, methyl bromide, formaldehyde, or ozone are broad antimicrobials and toxic to all life, yet their high reactivity, especially toward proteins preclude their broad use for biologics, transfusion products and in vivo. At the same time, their chemical reactivity makes them often inappropriate for many uses.

Targeting and inactivation of pathogens' nucleic acids is a universal approach to prevent pathogen replication and infectivity and can be applied to all classes of pathogens—viruses, bacteria, fungi, prions, protozoa and other parasites or undesirable organisms. Some existing methods utilize this approach by using intercalators, such as methylene blue, psoralen derivatives (U.S. Pat. Nos. 6,455,286 and 6,133,460) and riboflavin (U.S. Pat. No. 7,985,588), which selectively bind to the nucleic acids and, when photo-activated, damage them, thus exerting broad anti-pathogen activity. For instance, Estcourt et al., Jory et al., Magron et al. and Yonemura et al. describe pathogen inactivation in translucent blood components such as plasma and platelets by using photosensitizing compounds (Estcourt L J, Malouf R, Hopewell S, Trivella M, Doree C, Stanworth S J, Murphy M F), Pathogen-reduced platelets for the prevention of bleeding. Cochrane Database Syst Rev. 2017; 7:CD009072, doi: 10.1002/14651858.CD009072.pub3, PubMed PMID: 28756627; Joni G, Brown S B. Photosensitized inactivation of microorganisms. Photochem. Photobiol. Sci. 2004; 3(5):403-5, doi: 10.1039/b311904c. PubMed PMID: 15122355; Magron A, Laugier J, Provost P, Boilard E. Pathogen reduction technologies: The pros and cons for platelet transfusion. Platelets. 2018; 29(1):2-8, doi: 10.1080/09537104.2017.1306046, PubMed PMID: 28523956; Yonemura S, Doane S, Keil S, Goodrich R, Pidcoke H, Cardoso M. Improving the safety of whole blood-derived transfusion products with a riboflavin-based pathogen reduction technology. Blood Transfus. 2017; 15(4):357-64, doi: 10.2450/2017.0320-16, PubMed PMID: 28665269). A significant disadvantage of these methods is the need of photoactivation, which restricts their use to translucent compositions, only and precludes their use for such important biologics as whole blood or red blood cell preparations.

Alkylating compounds that inactivate pathogens, or other contaminants, by the alkylation of nucleic acids can be used to inactivate pathogens without the need of photoactivation. The challenge with this approach is to develop compounds which effectively penetrate the pathogen's cell walls, membranes and envelopes, and which possess enough selectivity in order to avoid modification of biologics proteins. Even the most selective representatives of alkylating pathogen inactivators, such as PEN110 (N-(2-aminoethyl)aziridine) and the alkylating intercalator 5303, have shown insufficient specificity toward nucleic acids and have residual reactivity toward other biological compounds (proteins for instance). This may result in the formation of neo-antigens when such alkylating agents are used for treatment of transfusable blood products (Sobral P M et al., Viral inactivation in hemotherapy: systematic review on inactivators with action on nucleic acids. Rev Bras Hematol. Hemoter. 2012; 34(3): 231-235, doi: 10.5581/1516-8484.20120056, PubMed PMID: 23049426; Conlan M G et al., Antibody formation to 5-303-treated RBCS in the setting of chronic RBC transfusion. Blood 2004; 104(11):382). Other monoaziridine-polyamine conjugates as antibacterials were disclosed in U.S. Pat. No. 6,617,157 and intercalating agents modified with alkylating moieties for selective targeting of pathogen's nucleic acids were disclosed in U.S. Pat. Nos. 6,410,219 and 5,691,132. The disadvantages of the disclosed structures and methods is that they do not achieve the necessary selectivity of nucleic acid targeting and do not avoid protein modifications.

U.S. Pat. No. 10,173,976, the disclosures of which are hereby incorporated by reference, describes compositions and compounds having two or more aziridinyl groups, interconnected through polyamine constructs, that have high and selective affinity to nucleic acids, low propensity to modify proteins, and can inactivate with a high selectivity the nucleic acids (e.g. DNA and/or RNA) of pathogens, pro-, or eukaryotes, or prion associated nucleic acids in a sample.

A drawback of this, and of other alkylating agents generally that target nucleic acids for use as pathogen inactivators, is that the residual alkylating compound (for example, in or on the organism, composition, sample, device, utensil, or utility) can be toxic, and cause harm either immediately after pathogen inactivation, or during subsequent use. This drawback can be addressed by removal of the anti-pathogen agent after the pathogen inactivation, or by its inactivation (quenching), i.e. conversion to less harmful or non-harmful substances.

U.S. Pat. No. 7,293,985, the disclosure of which are hereby incorporated by reference, describes the use of thiols, preferably glutathione, a dipeptide containing a cysteine residue, to quench in vitro a pathogen inactivating compound comprising a nucleic acids intercalator connected to a mustard type alkylating group, wherein the mustard group is capable of reacting in situ to form an electrophilic group. A disadvantage of this method is that it does not provide for sufficient inactivation of this type of nucleic acids targeting alkylation agent which results in neo-antigens and autoimmunity side effects when blood, treated by this method is infused in humans (Conlan M G et al., Antibody formation to S-303-treated RBCS in the setting of chronic RBC transfusion. Blood 2004; 104(11):382).

U.S. Pat. Appl. No. 20040137419, the disclosures of which are hereby incorporated by reference, describes a method for the removing of positively charged microbicidal compounds, and in particular PEN 110, N-(2-aminoethyl)aziridine, from treated compositions by using cation exchange resins.

U.S. Pat. No. 6,544,727, the disclosures of which are hereby incorporated by reference, describes methods and devices for the removal of psoralens and psoralen photoproducts formed after light irradiation from blood products. The methods include contacting a psoralen- and irradiation-treated blood product with a resin capable of adsorbing psoralens and psoralen photoproducts.

There is a need in the art for improved methods of pathogen inactivation that can be applied across a wide range of fields and applications, and particularly, methods of pathogen inactivation that spare proteins and other materials in the treatment sample; and for methods that leave little or no toxic compounds in the treated sample.

### SUMMARY OF THE INVENTION

In one aspect, the invention provides compositions and methods for the inactivation and/or reduction of pathogens, microorganisms, infectants such as prions, or parasites (e.g. contaminants) in a sample (including biological samples, media, compositions, utility, devices, surfaces, organisms, or the like) by treatment with an alkylating compound, followed by the elimination or reduction of the residual alkylating compound and/or its by-products. The elimination or reduction of the residual alkylating compound may be performed by treatment with a solid-phase agent, which reacts with, or otherwise sequesters the alkylating compound, or alternatively by treatment with a solution of a neutralizing compound, which eliminates or reduces the toxicity or other undesirable properties of the alkylating compound, preferably by eliminating its alkylating properties followed, in some instances, by removal of the products of neutralization of the alkylating compound and/or the excess of the neutralizing compounds by means of a solid phase agent that sequester them.

In one embodiment, the invention provides a method for inactivation or reduction of pathogens, microorganisms, infectants, or parasites (e.g. contaminants) in a sample comprising: (i) treatment of the sample with compound or compounds with Structure I:

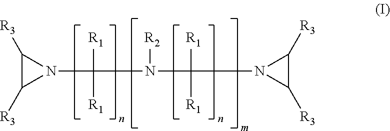

wherein:
each Ri is independently selected for each occurrence from H, Cl, F, an alkyl group, CH₃, CH₂CH₃, CH(CH₃)₂, an alkenyl group, a phenyl group, an alkyloxy group, an acyloxy group, or other substituted alkyl group,
each R₂ is independently selected for each occurrence from H, an alkyl group, CH₃, CH₂CH₃, CH(CH₃)₂, an alkenyl group, a phenyl group, a cycloalkyl group, an alkyloxy group, or substituted alkyl, alkenyl, cycloalkyl or phenyl group, or a moiety of Structure II:

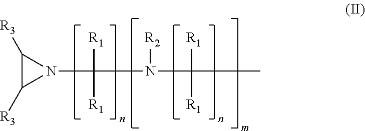

each R3 is independently selected for each occurrence from H, Cl, F, an alkyl group, CH₃, CH₂CH₃, CH(CH₃)₂, an alkenyl group, a phenyl group, an alkyloxy group, an acyloxy group, or other substituted alkyl group;
n is independently for each occurrence 3, 4, or 5;
m is independently for each occurrence 1, 2, 3, 4, 5, 6, 7, 8, 9 or 10;
or a chemically acceptable salt, hydrate, or solvate thereof, and
   (ii) elimination or reduction of the residual compound(s) having Structure I by treatment with a solid phase agent which reacts with, or otherwise sequesters the compound, or alternatively by treatment with a solution of a neutralizing compound which eliminates or reduces the toxicity or other undesirable properties of the compound with Structure I, preferably by eliminating its alkylating properties, followed, in some instances, by removal of the products of neutralization of the compound with Structure I and/or the excess of the neutralizing compounds by means of a solid phase agent that sequester them.

The compounds of Structure I contain at least two aziridine groups connected by polyamine constructs that binds with high affinity to nucleic acids and inactivate them by alkylation with high efficiency. In addition, the compounds penetrate with high efficiency viral envelopes and/or capsids, and are actively taken up by bacterial and eukaryotic polyamine transporters, and show low propensity for binding to and modifying proteins. Since the compounds of Structure I are cytotoxic to eukaryotic cells, they need to be rendered non-toxic or removed from the treated sample, composition, utility or organism.

In one embodiment, the method of the invention describes conversion of the residual compounds of Structure I to less toxic or non-toxic compounds by reaction with a neutralizing compound, which eliminates the alkylating properties of compounds of Structure I, for example, by opening the aziridine rings. The neutralizing compounds are nucleophilic compounds, such as thiosulfates, thiophosphates, thioureas, thiocarboxylic acids, dithiocarboxylic acids, thiocarbonate O-esters, dithiocarbonate O-esters, or mercaptans or thiols (preferably mercaptans or thiols that have pKa between 6 and 8, or in which the mercapto or thiol group is attached to a carbon atom in sp2, or partial sp2 hybridization).

In some instances, the products of neutralization (also called quenching) of compounds of Structure I or the residual neutralizing (quenching) compounds may themselves have undesired effect on the treated sample, or its future use. In another embodiment, the method involves the removal or reduction of the products of neutralization, and/or the neutralizing compound(s), by use of a solid phase agent which is insoluble in the treated media, and which either chemically reacts with, and covalently binds, absorbs, or otherwise sequesters the products of neutralization and/or the excess of the neutralizing compound(s), followed by removal of the solid phase agent. The solid phase agent may be functionalized with thiosulfate groups (—S—SO₃-Na+), or with epoxy groups, which react with and sequester mercaptan or thiol type of neutralizing compounds; or a solid phase agent that is a cationite or an anionite, which sequester through an ion-exchange the cationic type products of neutralization or anionic type of neutralizing compounds, or an absorbing solid phase agent, such as activated carbon that absorbs with high affinity polyamines or sulfur containing organic moiety.

In another embodiment of the method, after treatment of pathogen-containing samples with compounds of Structure I, the residual compounds are removed by treatment with a solid phase agent that contains reactive groups which react with and covalently bind the compound(s) of Structure I, followed by removal of the solid phase agent by filtration or other means. Examples of such reactive groups are thiosulfate, —OS(O)(O⁻)S⁻, thiosufonate —S(O)(O⁻)S⁻, mercapto or thiol groups, substituted mercapto or thiol groups, thioureas, thiocarboxylic or dithiocarboxylic acids, thiocarbonate or dithiocarbonate O-esters, thiophosphonate, or thiophosphates. The thiol groups may have a pKₐ less than 9 or, more preferably, less than 8. In another embodiment, the solid phase agent contains not only the reactive groups, but other groups, which without reacting with the compounds of Structure I, enhance their reactivity by protonating them, or non-covalently binding them, increasing their local concentration, or enhancing the reactivity of the reactive groups. In yet another embodiment, the solid phase agent contains non-reactive hydrophilic groups, such as polyethylene glycol, which improve its wettability in aqueous media and reduce its undesired effects on the components of the treated media.

Another embodiment describes the solid phase agent as a cationite, which forms multiple ion pairs with the residual compounds of Structure I thus retaining it in a highly efficient manner.

Some embodiments provide a method for inactivation of pathogens in animals or humans in vivo, where the compounds of Structure I, preferably formulated, are applied to the animal or human, and the neutralization or removal of the compounds of Structure I is done ex vivo on the bodily fluids, such as plasma or blood, which are then returned (transfused) back to the animal or human. In another embodiment, both the treatment with compound of Structure I and its removal, or its neutralization and possible removal of the neutralization products and the neutralizing compounds is done ex vivo on the bodily fluids of the animal or human, such as blood or plasma, preferably collected by apheresis, which are then returned to the animal or the human.

Also described herein are closed systems to be used according to the method for pathogen inactivation of whole blood, red blood cell or other blood products intended for transfusion.

### BRIEF DESCRIPTION OF THE FIGURES

FIG. 1 shows the interaction of a compound of Structure I with a solid phase agent having nucleophilic thiol groups attached through a linker L, and in which accessory anionic sulfo-groups are directly attached to the polymer P matrix.

FIG. 2 shows a whole blood unit processing closed-system for the collection of whole blood, in which pathogen inactivation is accomplished with a compound of Structure I formulated together with the anticoagulant solution in the blood collection bag, and removal of the residual compound of Structure I by passing of the treated blood through a cartridge containing a solid phase agent.

FIG. 3 shows a whole blood unit processing closed-system for the collection of whole blood, in which pathogen inactivation is accomplished with a solid formulation of compound of Structure I pre-loaded in a treatment bag and removal of the residual compound of Structure I by passing of the treated blood through a cartridge containing a solid phase agent.

FIG. 4 shows a whole blood unit processing closed-system for the collection of whole blood, in which pathogen inactivation is accomplished with a liquid formulation of a compound of Structure I and neutralization of residual compound with a liquid formulation of the inactivator.

FIG. 5 shows a whole blood unit processing closed-system for the collection of whole blood, in which pathogen inactivation is accomplished with liquid a formulation of a compound of Structure I and removal of the residual compound of Structure I by passing of the treated blood through a cartridge containing a solid phase agent.

FIG. 6 shows a whole blood unit processing closed-system for the collection of whole blood, in which pathogen inactivation is accomplished with a liquid formulation of a compound of Structure I, neutralization of the residual compound with a liquid formulation of the inactivator, and removal of the products of neutralization of the compound of Structure I with a solid phase agent.

FIG. 7 shows a whole blood unit processing closed-system for the collection of whole blood, in which pathogen inactivation is accomplished with a liquid formulation of a compound of Structure I, removal of the residual compound of Structure I with a solid phase agent, leukofiltration, and separation of the leukodepleated blood to red blood cells concentrate (RBCC) and plasma.

FIG. 8 shows a whole blood unit processing closed-system for the collection of whole blood, and leukofiltration, in which pathogen inactivation is accomplished with a liquid formulation of a compound of Structure I of the leukodepleted whole blood, removal of the residual compound of Structure I with a solid phase agent, and separation of the treated blood to red blood cells concentrate (RBCC) and plasma.

FIG. 9 shows a whole blood unit processing closed-system for the collection of whole blood, pathogen inactivation with liquid formulation of a compound of Structure I, two-stage removal of the residual compound of Structure I with a solid phase agent as free beads or prepacked in a semi-permeable material, leukofiltration, and separation of the leukodepleated blood to red blood cells concentrate (RBCC) and plasma.

FIG. 10 shows a whole blood unit processing closed-system for the collection of whole blood, pathogen inactivation with a solid formulation of a compound of Structure I, and neutralization of residual compound with a liquid formulation of the inactivator.

FIG. 11 shows a container containing a solid formulation of a compound of Structure I connected through a breakable seal to a container of the solvent for dissolving of the formulation and through another breakable seal to a container with the sample to be treated.

FIG. 12 shows a closed system for sterile pre-wetting of the solid phase agent packed in a cartridge.

FIG. 13 shows a closed system for rinsing of the solid phase agent before its use. The system is integrated in a closed system for treatment of a sample according the method under sterile conditions.

FIG. 14 shows the HPLC analysis of 10 μM 21-mer oligodeoxyribonucleotide (5′ ATA CCT CAT GGT AAT CCT GTT 3′ (SEQ ID NO:1)) incubated with 200 μM Compound X in PBS (pH 6.7) at 37° C. for 0 h (top), and 6 h (bottom).

FIG. 15 shows the mass-spectrometric analysis of the 23-mer oligonucleotide, 100 μM in PBS, before (top spectrum) and 6 min after (bottom spectrum) the addition of compound X (100 μM). The observed ions (m/z 1845.22 and 1933.54) are with charge state of minus 4, what corresponds to neutral molecules with masses of 7384.9 Da (oligonucleotide, calc. mass, 7384.0 Da) and 7738.2 Da (covalent mono-adduct of oligonucleotide with compound X, calc. mass, 7737.3 Da).

FIG. 16 shows the ESI+ mass-spectrometric analysis of cytochrome C, 8 μM, after incubation with compound X (top, 1 mM; middle, 100 μM; bottom, no compound X, control) for 30 hours at 40° C. The MS peaks from right to left correspond to 7×, 8×, 9×, 10× positively charged molecular ions of Cytochrome C.

FIG. 17A shows binding of mAb to non-treated (Ctr) and inactivated with 100 μM of compound VI or compound X respiratory syncytial virus (all were incubated for 4 hours at 40° C.).

FIG. 17B shows binding of mAb D25 to non-treated (Ctr) and inactivated with 100 or 500 μM compound VI respiratory syncytial virus (all were incubated for 6 hours at RT).

FIG. 18 shows the kinetics of neutralization of Compound X by ethyl 2-mercaptoacetate in PBS at RT. The concentration of compound X diminishes with a first order rate constant of 0.022 min⁻¹, and the concentration of intermediate Q1 XXI diminishes with a first order rate constant of 0.026 min⁻¹.

FIG. 19 shows the log plot of the concentration of compound VI during incubation with 1 mM sodium thiosulfate.

FIG. 20A shows a plot of the rate of neutralization of compound X and the rates of formation of compounds XXIV and XXV. FIG. 20B shows a logarithmic plot of compound X concentration, which reveals a liner dependence, indicating a first order reaction kinetics with a first order rate constant K=−0.0416 min⁻¹, corresponding to compound X half-life of T_{1/2}=16.6 min.

FIG. 21 shows the mass chromatogram of the LCMS analyzes of the neutralization of compound X with thiophenol after 100 second incubation (left panel). Mass spectra of the peaks corresponding to compound X and its neutralization products XXVI and XXVII are shown in the right panel. The analysis reveals that after 100 seconds, compound X is neutralized by a significant degree.

FIG. 22A shows the effect of mock-treated and Compound VI-treated serum on the growth of porcine PT cells in 48-well plates measured over a 6-7-day period. The TO column indicates cell numbers in time of plating; First columns in array of three (day 1 to 7) is the number of cells in wells containing medium supplemented with control, non-treated serum; Second columns in array of three (day 1 to 7) is the number of cells in wells containing medium supplemented with mock-treated serum; Third columns in array of three (day 1 to 7) columns is the number of cells in wells containing medium supplemented with Compound VI-treated serum. Each time point represents the mean of three wells. Error bars indicate the SD.

FIG. 22B shows the effect of mock-treated and Compound VI-treated serum on the growth of human A172 cells in 48-well plates measured over a 6-7-day period. The TO column indicates cell numbers in time of plating; First columns in array of three (day 1 to 7) is the number of cells in wells containing medium supplemented with control, non-treated serum; Second columns in array of three (day 1 to 7) is the number of cells in wells containing medium supplemented with mock-treated serum; Third columns in array of three (day 1 to 7) columns is the number of cells in wells containing medium supplemented with Compound VI-treated serum. Each time point represents the mean of three wells. Error bars indicate the SD.

FIG. 22C shows the effect of mock-treated and Compound VI-treated serum on the growth of human MCF-7 cells in 48-well plates measured over a 6-7-day period. The TO column indicates cell numbers in time of plating; First columns in array of three (day 1 to 7) is the number of cells in wells containing medium supplemented with control, non-treated serum; Second columns in array of three (day 1 to 7) is the number of cells in wells containing medium supplemented with mock-treated serum; Third columns in array of three (day 1 to 7) columns is the number of cells in wells containing medium supplemented with Compound VI-treated serum. Each time point represents the mean of three wells. Error bars indicate the SD.

FIG. 22D shows the effect of mock-treated and Compound VI-treated serum on the growth of bovine BTT cells grown in medium with FBS in 48-well plates measured over a 6-7-day period. The TO column indicates cell numbers in time of plating; First columns in array of three (day 1 to 7) is the number of cells in wells containing medium supplemented with control, non-treated serum; Second columns in array of three (day 1 to 7) is the number of cells in wells containing medium supplemented with mock-treated serum; Third columns in array of three (day 1 to 7) columns is the number of cells in wells containing medium supplemented with Compound VI-treated serum. Each time point represents the mean of three wells. Error bars indicate the SD.

FIG. 22E shows the effect of mock-treated and Compound VI-treated serum on the growth of bovine BTT cells grown in medium with HS in 48-well plates measured over a 6-7-day period. The TO column indicates cell numbers in time of plating; First columns in array of three (day 1 to 7) is the number of cells in wells containing medium supplemented with control, non-treated serum; Second columns in array of three (day 1 to 7) is the number of cells in wells containing medium supplemented with mock-treated serum; Third columns in array of three (day 1 to 7) columns is the number of cells in wells containing medium supplemented with Compound VI-treated serum. Each time point represents the mean of three wells. Error bars indicate the SD.

### DETAILED DESCRIPTION OF THE INVENTION

The term “sample” as used herein refers to a media, composition, product, device, utility or organism that can be prokaryotic, single or multicellular eukaryotic, plants, animal, blood or blood products, bodily fluids, medium originated from eukaryotes or prokaryotes, vaccine preparation compositions, biologics or biologic preparations, clinical sample, biopsy, research sample, cosmetics, pharmaceutical compositions, disposables, instrument, aquatic fluid conduits, pipes, hoses, heat exchanges, or aquatic vessels and their surfaces.

The terms neutralizer, neutralizer compound or neutralizer agent, when used in the context of compound(s) of structure I, designate molecules that, in general, can react and open aziridinyl groups of the compounds of Structure I in a sample.

The term “solid phase agent” used in the context of the methods described herein is defined as a solid that is insoluble in the media of the sample, and that is used to remove the compound of structure I, or the products of inactivation of compound of structure I, or the products of chemical transformation or degradation of the compounds of structure I or the neutralizing agent from the sample.

The term “contaminant” as used herein refers to pathogens, including viruses, bacteria, or any other microorganisms, prions, or eukaryote, single-, or multicellular eukaryote, including, but not limited to fungi, protozoa, single- or multicellular parasite including helminths, schistosomes or nematodes or their eggs, single or multicellular algae and of crustacean, or any other undesirable organisms or infectants. The term “contaminant” as used herein can also refer to undesirable biological structures, including without limitation, bacterial biofilms or other microorganism biofilms, lichens, encrustations or biofouling accumulations.

The invention provides a method for contaminant inactivation/reduction in a sample by treatment with compound of Structure I followed by removal or neutralization (quenching) of the residual compound of Structure I:

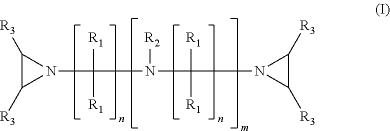

wherein:
each R₁ is independently selected for each occurrence from H, CH₃, CH₂CH₃, CH(CH₃)₂, Cl, F, an alkyl group, an alkenyl group, a phenyl group, an alkyloxy group, an acyloxy group, or substituted alkyl group,
each R2 is independently selected for each occurrence from H, CH₃, CH₂CH₃, CH(CH₃)₂, an alkyl group, an alkenyl group, a phenyl group, a cycloalkyl group, an alkyloxy group, or substituted alkyl, substituted alkenyl, substituted cycloalkyl or substituted phenyl group, or a moiety of Structure II:

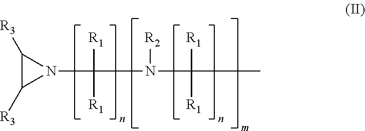

each R3 is independently selected for each occurrence from H, CH₃, CH₂CH₃, CH(CH₃)₂, Cl, F, an alkyl group, an alkenyl group, a phenyl group, an alkyloxy group, an acyloxy group, or other substituted alkyl group;
each n is independently for each occurrence 3, 4, or 5;
each m is independently for each occurrence 1, 2, 3, 4, 5, 6, 7, 8, 9 or 10;
   or a chemically acceptable salt, hydrate, or solvate thereof.

In some embodiments, the compound of Structure I may have the Structure IA:

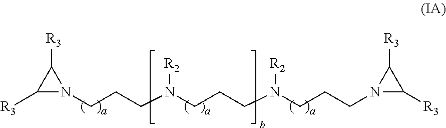

wherein:
each R₂ is independently selected for each occurrence from H, an alkyl group, CH₃, CH₂CH₃, CH(CH₃)₂, an alkenyl group, a phenyl group, a cycloalkyl group, an alkyloxy group, or substituted alkyl, alkenyl, cycloalkyl, phenyl group, or a moiety of Structure IIA:

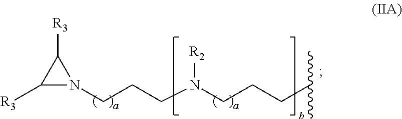

each R₃ is independently selected for each occurrence from H, Cl, F, an alkyl group, CH₃, CH₂CH₃, CH(CH₃)₂, an alkenyl group, a phenyl group, an alkyloxy group, an acyloxy group, or a substituted alkyl group;
each a is independently selected for each occurrence from 1, 2 or 3; and
each b is independently selected for each occurrence from 0, 1, 2, 3, 4, 5 or 6.

In some embodiments, the compound of Structure I may have the Structure IB:

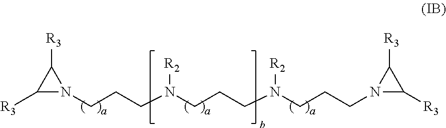

wherein
each R₂ is independently selected for each occurrence from H, CH₃, CH₂CH₃, or CH(CH₃)₂;
each R₃ is independently selected for each occurrence from H, CH₃, CH₂CH₃, or CH(CH₃)₂;
each a is independently selected for each occurrence from 1, 2 or 3; and
b is selected from 0, 1, 2, 3, 4, 5 or 6.

The term “alkyl” refers to the radical of saturated aliphatic groups, including straight-chain alkyl groups and branched alkyl groups. In preferred embodiments, a straight chain or branched chain alkyl has 6 or fewer carbon atoms in its backbone (e.g., C₁-C₆ for straight chain, C₃-C₆ for branched). Preferred alkyl groups include CH₃, CH₂CH₃, CH₂CH₂CH₃ and CH(CH₃)₂.

The term “substituted alkyl” refers to an alkyl group as provided above which is substituted by 1 to 3 substituents which are independently selected from the group consisting of F, Cl, OH, OCH₃, OCH₂CH₃, OCH(CH₃)₂, OC(CH₃)₃, 006H₅, OCOCH₃.

The term “cycloalkyl” refers to saturated, carbocyclic groups having from 3 to 6 carbons in the ring. Preferred cycloalkyl groups include cyclopropyl, cyclobutyl, cyclopentyl and cyclohexyl.

The term “alkenyl group” refers to a radical of unsaturated aliphatic groups, including straight-chain alkenyl groups and branched alkenyl groups, and having 1 to 3 double bonds. In preferred embodiments, a straight chain or branched alkenyl has 6 or fewer carbon atoms in its backbone (e.g., C₂-C₆ for straight chain, C₃-C₆ for branched).

The term “substituted alkenyl” refers to an alkenyl group as provided above which is substituted by 1 to 3 substituents which are independently selected from the group consisting of F, Cl, OH, OCH₃, OCH₂CH₃, OCH(CH₃)₂, OC(CH₃)₃, 006H₅, OCOCH₃.

The term “substituted phenyl” refers to a phenyl group which is substituted by 1 to 3 substituents which are independently selected from the group consisting of F, Cl, OH, OCH₃, OCH₂CH₃, OCH(CH₃)₂, OC(CH₃)₃, 006H5, OCOCH₃.

The term “alkyloxy group” refers to an alkyl group, as defined above, which is attached through an oxygen atom. Representative alkyloxy groups include methoxy, ethoxy, propyloxy, tert-butoxy and the like.

The term “acyloxy group” refers to a group having the structure —O—(C═O)—R, in which R is an alkyl group or a substituted alkyl group as provided above.

As used herein, the definition of each expression, e.g. alkyl, m, n, R₁, R₂, R₃, etc., when it occurs more than once in any structure, is intended to be independent of its definition elsewhere in the same structure.

It will be understood that “substituted” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As set out above, in certain embodiments the compounds of Structure I are present as salts. Preferred salts are relatively non-toxic, inorganic and organic acid addition salts of compounds of Structure I. These salts can be prepared in situ in the administration vehicle, or by separately reacting a purified compound of Structure I in its free base form with a suitable organic or inorganic acid, and isolating the salt thus formed during subsequent purification. Representative salts include the hydrobromide, hydrochloride, sulfate, bisulfate, phosphate, perchlorate, tetrafluoroborate, nitrate, acetate, valerate, oleate, palmitate, stearate, laurate, benzoate, lactate, phosphate, tosylate, citrate, maleate, fumarate, succinate, tartrate, napthylate, methansulfonate, glucoheptonate, lactobionate, and laurylsulphonate salts and the like (see, e.g., Berge et al. (1977) “Pharmaceutical Salts”, J. Pharm. Sci. 66:1-19). Preferably the anion has a low nucleophilicity, such as sulfate, perchlorate, methansulfonate or tetrafluoroborate.

The compounds of Structure I are of polyamine nature, having two or more aziridinyl groups on their termini. These compounds have multiple aliphatic nitrogen atoms that can each be positively charged in vitro or in vivo. Due to their polycationic nature and the appropriate spacing between the positive charges, the compounds selectively bind to the polyanionic nucleic acids and alkylate them, preferably on guanine N7 positions. This results in cross linking, effectively inactivating the pathogen's genome, eliminating pathogen's infectivity or killing the organism.

The compounds having Structure I can be synthesized by the methods disclosed herein. The following schemes, such as the synthesis of the compositions and compounds, are provided for illustrative purposes and are in no way intended to limit the scope of the present invention. One of ordinary skill in the art can readily appreciate different chemical approaches and synthetic schemes of the compounds of Structure I.

Methods of the synthesis of compounds of Structure I are provided in the following schemes.

Scheme 1 shows a method for the preparation of compound IV:

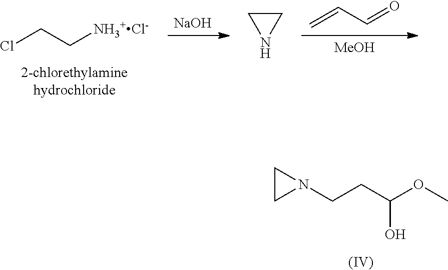

Scheme 2 shows a method for the preparation of compound VI:

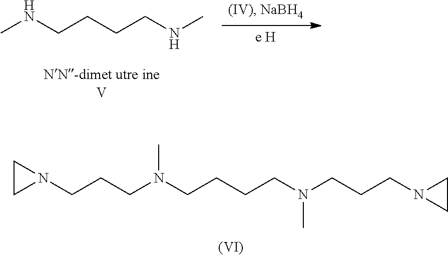

Scheme 3 shows a method for the preparation of compound X:

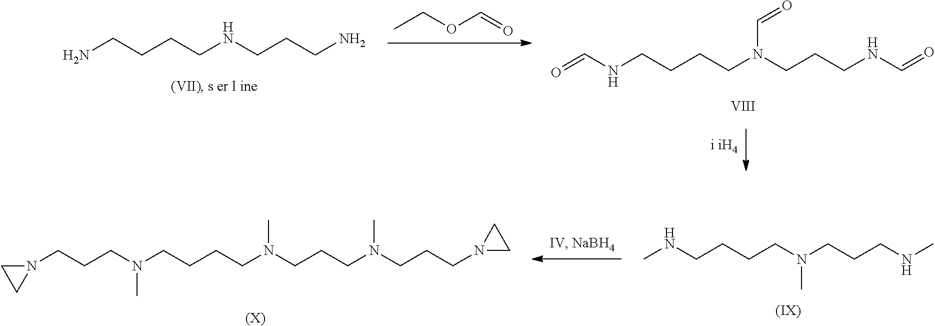

Scheme 4 shows a method for the preparation of compound XIV:

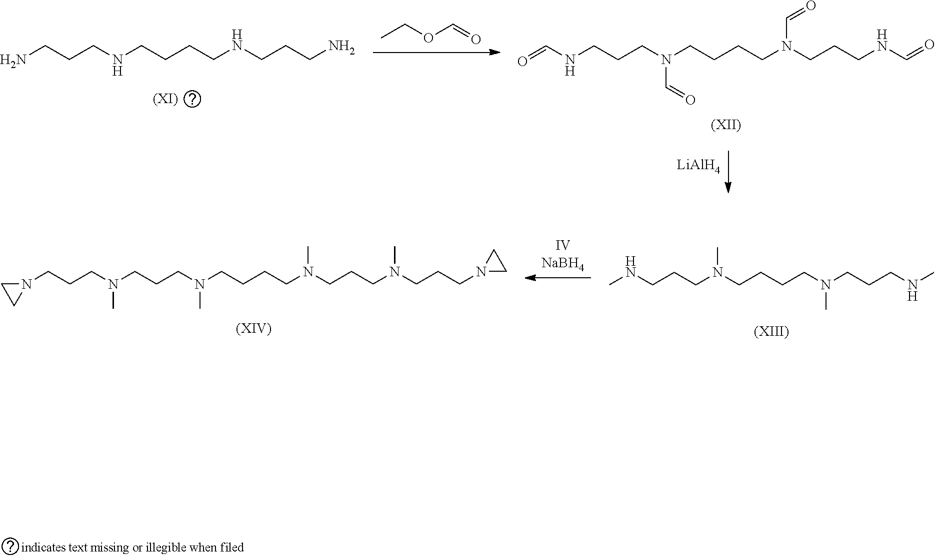

Scheme 5 shows a method for the preparation of compound XVI:

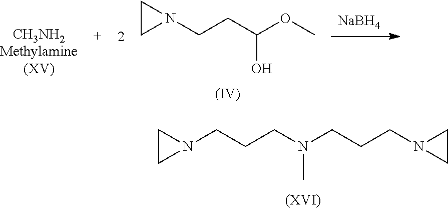

Generally, the compounds of Structure I are viscous oils, which are well soluble in water, aqueous buffers and organic solvent. They can be converted to the salt form if treated with acids. If their solutions in non-polar aprotic solvents, such as ether, are treated with a stoichiometric amount of anhydrous acid, preferably at low temperatures, their salts may be precipitated and may be isolated by filtration. In some embodiments of the present invention, the salt forms are used for long term storage instead of the free base, oil forms.

The solutions of the free bases of the compound of Structure I are alkaline, and can absorb atmospheric carbon dioxide, which can compromise the stability of the solutions and accelerate their hydrolysis or other degradation. The free bases of the compounds of Structure I may be stabilized by addition of small amounts of basic compounds, for example of sodium hydroxide. For instance, the glycerol solution of compound X is significantly stabilized to long term storage by addition of 0.1% of sodium hydroxide.

The compounds of Structure I can be converted to solid solutions by quick solidification by cooling of their solution in compounds which are solid at room temperature. For example, if compound VI is added, in amount of up to 3% to melted polyethylene glycol, and the resulted solution is cooled quickly, preferably in thin film, a solid solution of compound VI is formed. This solution has significantly higher storage stability than the neat compound VI. The stability of the solid solutions can be further enhanced by addition of traces of strong bases, as for example, of sodium hydroxide. The preferred solids for the preparing of solid solution of compounds of Structure I have melting points above 40° C. and below 120° C., are well soluble in aqueous media, are neutral in chemical character, and have no adverse effect on the sample to be treated by the process, or on its intended use.

Our experiments and the data presented in the examples of this invention show that representative compounds of Structure I quickly form covalent adducts with RNA and DNA oligonucleotides, and inactivate high titer of various pathogens (enveloped and non-enveloped, DNA and RNA viruses, G+ and G− bacteria, mycoplasma, fungi and protozoa) in different kind of media, such as growth media, whole blood, red blood cell concentrate, plasma and serum, at low concentrations (100-500 μM) and at different temperatures (20 to 40° C.).

According to the method of the present invention the contaminants in the sample are treated with neat compound of Structure I, or with a composition containing one or more compounds of Structure I, where the composition can be formulated as a liquid, solution, gel, solid, powder, particles, or can be encapsulated, dissolved, dispersed, pulverized, micronized, or converted to nano-particles, or in other formulated forms or in combinations thereof. The solvent for the compositions of the compounds of Structure I may water, aqueous buffers, or aqueous salt solutions, organic solvents, such as, but not limited to, dimethylsulfoxide, dimethylacetamide, ethanol, iso-propanol, acetone, polyethylene glycol(s) of different molecular masses, glycerol, propylene glycol, benzyl alcohol, or mixtures thereof, liquidities gasses, or mixtures thereof. The solvents can contain various organic or inorganic additives, stabilizers, activators, or adjuvants.

In embodiments of the present invention, the sample containing a contaminant is treated with compound(s) with Structure I for a period of time from 30 sec to 72 hours, preferably from 20 min to 24 h and even more preferably from 60 min to 8 h, and at temperatures from 0 to 100° C., preferably from 10 to 60° C., and even more preferably from 20 to 40° C.; and at pH from 1 to 14, preferably from 4 to 9 and even more preferably from 6 to 8; and at concentrations from 10 nM to 10 mM, preferably from 1 μM to 1 mM, still more preferably from 100 μM to 500 μM.

The contaminant inactivation effect of the compound(s) of Structure I increases with the increase of their concentration, dose or amount, treatment time, and temperature. At the same time, possible undesired effect on the treated sample also may increase with the compound concentration, dose or amount, time and temperature of treatment. The user of the method can determine the optimal concentration, dose or amount of compound(s) of Structure I, time and temperature of treatment based on the type and properties of the treated media and the nature and type of pathogens or undesired organisms present into it, and the desired level of their inactivation. For example, utilities that are stable to temperature, such as biofouling heat exchangers, can be treated at elevated temperature, for instance 60° C. and up, and for extended periods of time, for instance 24 h and more. At the same time, the optimal treatment temperature for a sensitive sample, such as for instance, platelets concentrate may be room temperature, and the treatment time may be restricted to 1-2 h or less, while for heat tolerant samples, such as heat-treated animal sera, the optimal temperature may be 40° C. or more, at a treatment time of 1-6 h. The user can determine the optimal concentration, dose, or amount of compound(s) of Structure I, and the time and temperature of treatment by experimentation, using the approaches disclosed herein, and similar approaches known to one skilled in the art.

The optimal treatment parameters (concentration, time, temperature) may depend not only on the properties of the treated sample and the type and nature of the pathogens or other undesired organisms present in it, but also on the desired degree of their inactivation/reduction, which may depend on the intended use of the treated sample. For example, if the treated sample is animal sera with intended use as supplement to cell growth media, the required level of viruses that can infect that cells may be below one infectious particle per used dose, which may require reduction/inactivation level of more than 9 logs, whereas if the treated utility is industrial piping with the purpose controlling of biofilm formation or biofouling, one or two logs of microorganism reduction may be sufficient.

The method of the invention provides, through selecting the compound(s) of Structure I and the treatment parameters (concentration/dose/amount, time, temperature, pH, formulation) means for pathogen(s) or undesired organisms, and in some cases, to all pathogens or undesired organisms present into the sample inactivation form 50% to up to full sterilization of the treated sample.

On the other hand, the structure, mechanism of action, and our experiment, indicate that compounds of Structure I are cytotoxic, and should be removed, or their cytotoxicity abrogated, for the safe use of the treated sample or for the safety of the treated organism.

In some embodiments of this invention, the alkylating properties of the compounds of Structure I, and therefore their cytotoxicity resulting from those alkylating properties can be reduced or removed by treatment of the sample, where residual compound of Structure I is present, with small nucleophilic molecules or ions, such as, but not limited to, thiosulfate, preferably sodium thiosulfate, thiophosphate, preferably sodium thiophosphate, thiourea or substituted thioureas, such as monomethyl-, N,N- or N,N′-dimethyl-, trimethyl-, or tetramethylthiourea, thiocarboxylic acids, such as thioacetic acid (CH₃C(O)SH), thiopropionic acid, thiooxalic acid, thiomalonic acid, thiosuccinic acid, dithiocarboxylic acids, such as dithioacetic acid (CH₃C(S)SH), thiocarbonate O-esters, such as ethyl thiocarbonate, dithiocarbonate O-esters, such as ethyl dithiocarbonate, or mercaptanes, or thiols, such as, but not limited to, 2-mercaptoethanol, 3-mercaptopropane-1,2-diol (1-thioglycerol), 2-thioglycerol, 1,2- or 1,3-dithioglycerol, 2-aminoethanethiol, 2-(methylamino)ethanethiol, 2-(dimethylamino)ethanethiol, 2-mercapto-N,N,N-trimethylethanaminium salts, (methyl sufony)methanethiol, (ethyl sulfonyl)methanethiol, sulfonyldimethanethiol, thioglycolic acid (HSCH₂CO₂H), 2-mercaptosuccinic acid, aromatic or heterocyclic thiols, such as thiophenol, furan-2-thiol, 2-thiopyridine, 1H-imidazole-2-thiol, 1H-imidiazole-5-thiol, thiobarbituric acids, thiosalicylic acid or 4-mercaptobenzoic acid. Some examples of preferred thiol compounds are presented below:

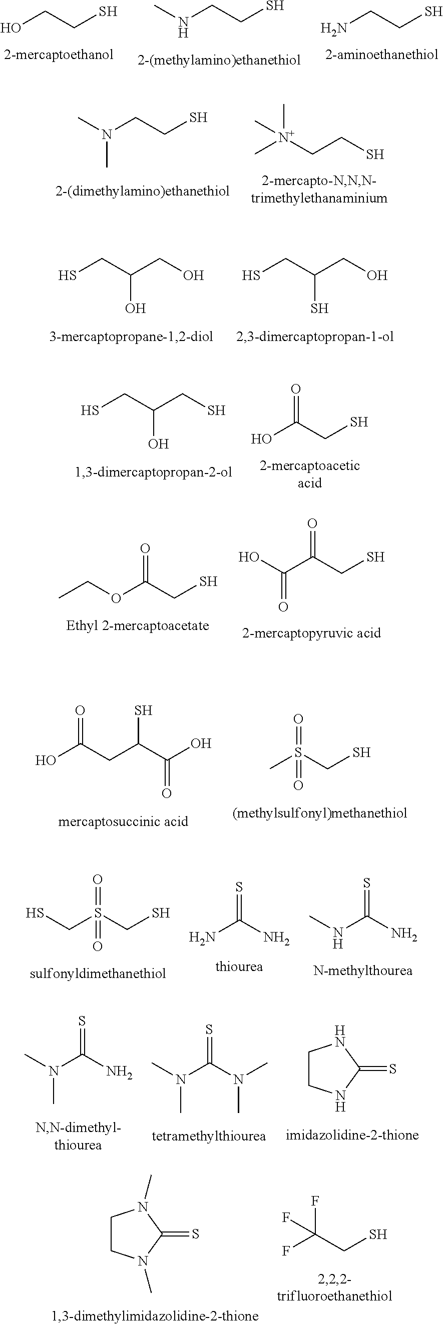

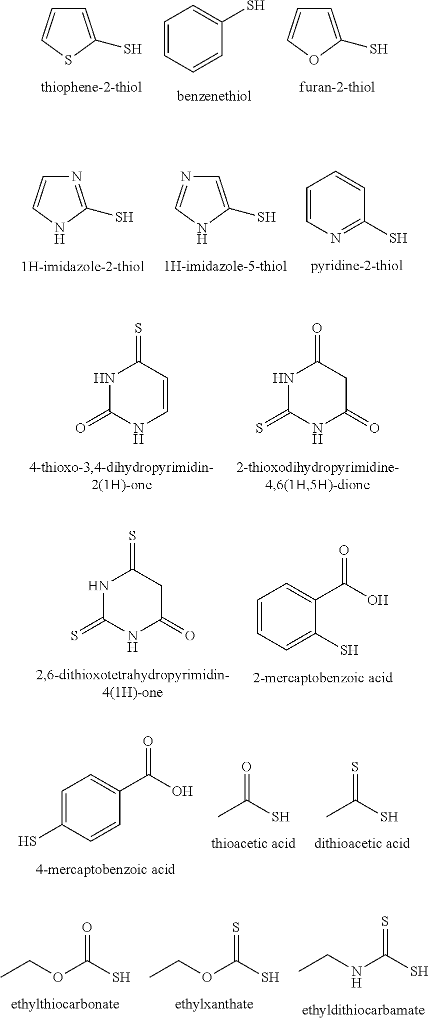

As it is shown in the examples, the small nucleophilic molecules react with the compounds with Structure I by opening their aziridine rings, thus eliminating their ability to alkylate nucleic acids. The rate of this reaction depends on the temperature, pH, and concentration, and on the nucleophilicity of the small nucleophilic molecules.

The nucleophilicity of the thiols increases significantly with the deprotonation of the thiol, i.e. their nucleophilicity is mainly due to the deprotonated, anionic form of the thiol (Danehy, J. P.; Noel, C. J. The Relative Nucleophilic Character of Several Mercaptans toward Ethylene Oxide. Journal of the American Chemical Society 1960, 82, 2511-2515). In general, the nucleophilicity of anionic nucleophiles of the same type, and in particular, the nucleophilicity of the thiol nucleophiles increases with their basicity, i.e. nucleophiles with higher pKₐ will have more nucleophilic anionic form than nucleophiles with lower pKₐ (more acidic nucleophiles). At the same time the concentration of the deprotonated (anionic) from of a nucleophile decreases with the increase of the difference between the pKₐ of the nucleophile and the pH of the medium, i.e. decreases with the increase of the nucleophile pKₐ at above the pH of the medium.

In some embodiments of this invention, the preferred thiol type of neutralizer of the compounds of Structure I have pKₐ close to the pH of the media in which the inactivation takes place, i.e., if the neutralization takes place at pH 7, or close to pH 7, the preferred thiol type neutralizer have pKₐ close to 7, which will provide a best compromise between the increase of the nucleophilicity of the anionic form of the neutralizer with the increase of its basicity and the decrease of the concentration of the anionic form with the increase of its pKₐ above the pH of the media. This teaching is supported by our experiments, in which the half-life of one representative of compounds of Structure I with formula X in presence of 10 mM of thiophenol (pKₐ=6.52) was determined to be below 1 min, whereas the half-life of the same compound under the same conditions in presence of glutathione (pKₐ of SH group=8.75) was 450 min.

In another embodiment of the present invention, the preferred thiol type of neutralizer of the compounds of Structure I has a thiol group which is directly attached to a carbon atom which is a part of a double bond, or an aromatic system, or has full or partial sp² type of hybridization.

In yet another embodiment, the preferred thiol type of neutralizer of the compounds of Structure I has at least one electron-accepting group, such as sulfone group (—S(O₂)—R), or sulfoxide group (—S(O)—R), or ester group (—C(O)OR) or amide group (—C(O)NH₂, —C(O)NHR, —C(O)NR₂), where R is any alkyl or substituted alkyl group, which electron-accepting group is attached to the carbon atom to which the SH group is attached.

In some embodiments of the invention, the residual compound(s) of Structure I in the treated sample, composition, surface, device or organism are neutralized by contacting with the neutralizing compound(s) or with solutions of the neutralizing compound(s) in appropriate solvent(s), such as, but not limited to, water, aqueous buffer or aqueous salts solutions, organic solvent, such as, but not limited to, dimethylsulfoxide, dimethylacetamide, ethanol, iso-propanol, acetone, polyethylene glycol(s) of different molecular masses, glycerol, propylene glycol, benzyl alcohol, or mixtures thereof for the time necessary for the desired neutralization or degree of neutralization to take place, preferably for less than 72 h, more preferably for less than 24 h, and even more preferably for less than 8 h, and yet even more preferably for less than 4 h, and at temperatures from 0 to 100° C., preferably from 10 to 60° C., and even more preferably from 20 to 40° C., and at pH from 1 to 14, preferably from 4 to 9 and even more preferably from 6 to 8. The concentrations of the neutralizing compound in the treated sample can be up to 1 M, preferably up to 0.1 M, and even more preferably up to 10 mM.

It is understood that the optimal conditions for the fastest and most efficient neutralization of the residual compound of Structure I in the treated media is different and depend on the type of media, and the type of neutralizing compound, and they can be reasonably selected and optimized experimentally by a person with ordinary skill in the art by using the disclosed hereby or similar experimental methods.

The desired neutralization, or degree of reduction of the amount of the residual compound(s) of Structure I is less than 50%, preferably more than 2 times, even more preferably by more than 10 times, i e, 1 log, and even more preferably by more than 2 logs, still more preferably by at least 3 logs, and still more preferably by at least 4 logs, still more preferably by at least 5 logs, still more preferably by at least 6 logs, still more preferably by at least 7 logs, still more preferably by at least 8 logs, still more preferably by at least 9 logs, still more preferably by at least 10 logs or more.

In some embodiments of the invention the product(s) of neutralization of the compound(s) with Structure I, i.e., the products of their reaction with the neutralizing compound(s), or the products of reaction of compounds with Structure I with the components of the treated sample may have undesired properties for the intended use. In other cases, the neutralizing compounds may have undesirable properties. In all those cases, the products of neutralization or products of reaction, or the neutralizing compounds can be removed from the treated sample, or their amount can be reduced, by treatment of the sample with a solid phase agent which is insoluble in the treated media, and which solid phase agent chemically reacts with and covalently binds, or absorbs, or otherwise sequesters the products of neutralization or reaction of the compound(s) of Structure I and/or the neutralizing compound(s). After the treatment, the solid phase agent can be removed from the treated media by filtration, centrifugation, sedimentation or other appropriate physical means. Alternatively, the solid phase agent may be in contact with the treated media through a membrane, pouch or other appropriate physical barrier, which is permeable by the products of neutralization or the products of reaction of the compound(s) of Structure I with the components of the treated sample, or the neutralizing compound(s) and is not permeable by the solid phase agent.

The solid phase agent may be a porous organic polymer of micro-, or macroporous, or gel type, or it can be any highly porous solid of organic or inorganic type, such as, but not limited to amorphous carbon, activated carbon, charcoal, silica gel, titania, circonia, or it may be a non-porous solid with high dispersity, i.e., of small particle size that provides for high surface to volume ratio. The solid phase agent may also be of mixed type, for instance, solid non-porous particles, which are covered with a layer of porous material.

The organic polymer, preferably cross-linked, can be a polystyrene polymer, or polyacrylate polymer, or polymethacrylate polymer, or polyurethane polymer, or polyamide polymer, or dextran polymer, such as, but not limited to Sephadex®, or agarose polymer, such as but not limited to Sepharose®, or a cellulose based polymer, or modified cellulose based polymer, such as but not limited to carboxymethylcellulose, or diethylaminoethyl cellulose, or methylcellulose, or other polysaccharide, or any other linear, branched, or cross-linked homo- or hetero-polymer or block copolymer, with iso- or atactic configuration, or with other tacticity, or may be any other appropriate macromolecule that is not soluble in the treated media.

For the treatment of aqueous based media, a hydrophilic organic polymer, or polymer which is wettable, or can expand, or swell in aqueous based media is highly preferred.

In some embodiments the solid phase agent chemically reacts with, and covalently binds the products of neutralization or reaction of the compound(s) of Structure I and/or the neutralizing compound(s). For example, epoxy-modified resins, such as epoxy-modified polyacrylate resins, such as Lifetech™ ECR8215M, or epoxy-modified agarose resin, such as Praesto® Epoxy300, both resins manufactured by Purolite Ltd, Bala Cynwyd, PA, USA, react easily with nucleophilic compounds, and specifically with the nucleophilic compounds used as neutralizers of the compounds of Structure I in this disclosure, as for example with sodium thiosulfate as disclosed by Axen et al. in Preparation of modified agarose gels containing thiol groups, Acta Chem. Scand. B 1975, 29, 471. In this reaction the nucleophilic neutralizer opens the epoxy ring and attaches covalently to the polymer molecule. In another example, polymers, functionalized with functional groups containing electrophilic sulfur atom, such as S-methanesulfonates (P—S—S(O₂)CH₃, where P denotes the polymer molecule), or S-thiosulfate esters (P—S—S(O₂)O⁻M⁺, where P denotes the polymer molecule, and M denotes metal cation) react easily with thiols, such as the neutralizers of the compounds of Structure I of thiol type according to the reaction:

(P—S—S(O₂)O⁻M⁺+RSH→P—S—SR+M+SO₃²⁻

resulting in attachment of the thiol type neutralizer to the polymer trough a disulfide bond. Those type of polymers, their preparation, and the reactions are disclosed by Roth and Theato in RSC Polymer Chemistry, Ser. 6 (2013): Thiol-X in Polymer and Material Science, Chapter 4: Thiol-Thiosulfonate Chemistry in Polymer Science, pages 76-94 and in the references cited therein, all of which are incorporated herein by reference. If the treated sample contains proteins, or other macromolecules that can react with the electrophilic functional groups of the solid phase agent, the solid phase agent is contacted with the matrix through a semi-permeable membrane, which is permeable for small molecules and impermeable for macromolecules, such as dialysis membranes with cut-off of from 1000 to 10000 Da.

In another embodiment, the solid phase agent absorbs the products of neutralization or products of degradation or products of reaction with the matrix components of the compound(s) of Structure I and/or the neutralizing compound(s). Example of such type of solid phase agent is activated carbon, or charcoal, which absorbs with high affinity polyamine type of compounds (Cohen, S. S., A Guide to the Polyamines, Oxford Univ. Press, 1988), and also absorbs with high affinity sulfur containing organic compounds, such as the thiol type of neutralizers, such as, but not limited to, thiophenol, thioanisole, furan-2-thiol, thiosalicylic acid, 4-thiobenzoic acid, dithioacetic acid, or thioglycolic acid.

In another embodiment the solid phase agent absorbs the products of neutralization or reaction of the compound(s) of Structure I by forming multiple ion pairs with them. The compounds of Structure I, the products of their neutralization, and the products of their decomposition or reaction with the matrix components have multiple (more then 3) aliphatic nitrogen atoms, which atoms are protonated at neutral or acidic pH. In result of that, the compounds are polycationic, i.e. they have 3 or more positive charges at neutral, close to neutral, or at acidic pH.

The solid phase agents that contains multiple negatively charged groups can form multiple ion pairs with the polycationic compounds and absorb them through electrostatic interactions. Such solid phase agents can be cation exchange resins, such as strong cation exchange resins, preferably sulfo-groups or sulfate-groups containing cation exchange resins, or weak cation exchange resins, preferably carboxy-groups containing cation exchange resins. Examples of such cation exchange resins are Dowex® 50X2-200, Amberlite® IR-120 of Dow Chemicals, or NRW160 of Purolite.

The exchangeable cation associated with the cation exchange resins is selected to be compatible with, or not-detrimental to the sample or its use, and is preferably sodium for biological materials. The ion-exchange capacity of the resin should be at least 0.01 meq/ml, preferably, at least 0.1 meq/ml and even more preferably, at least 1 meq/ml.

There are numerous types of cation exchange resins, based on different polymer type, degree of cross-linking, degree of functionalization and porosity, and degree of purity and leachables release. One with ordinary skills in the art is be able to select an ion-exchange resin which is compatible with, and does not present harmful effect to the treated media, and at the same time have high degree of functionalization and retention of the neutralized compounds.

In another embodiment of the present invention, excess of neutralizers of compound of Structure I of the anionic type, such as thiosulfate, thiophosphate, thiocarboxylic acids, thioacetate, thioglycolate, thiolactate, dithiocarboxylic acid salts, 2-mercaptoacetate, 2-mercaptosuccinate, 2-mercaptopropionate, thiosalicylic acid, 4-mercaptobenzoic acid are removed from the treated sample or media by using a solid phase agent which has multiple cationic groups covalently attached to it, such as an anion-exchange resin. The anion-exchanger may be a weak, but is preferably a strong anion exchanger, and may have primary, secondary, or tertiary amino groups or quaternary ammonium group attached to it, which are ion-paired with appropriate anionic groups, such as, but not limited to, chloride, sulfate, succinate, lactate or other cationic groups that are compatible with and not detrimental to the treated sample and to its properties.

In an embodiment of the present invention, after contaminant inactivation by treatment with compounds of Structure I, the residual compound(s) of Structure I are removed from the sample by treatment with a solid phase agent which reacts with and covalently binds the compound(s) of Structure I. The solid phase agent can contain reactive groups that react with, and open the aziridine rings of the compound(s) of Structure I. The solid phase agent is with general structure XVII:

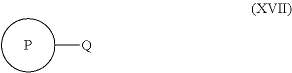

wherein
Q is a reactive group that chemically reacts and covalently binds the compound(s) of structure I; and
P is the solid phase agent matrix, which can be a porous organic polymer of micro-, or macroporous, or gel type, or it can be any highly porous solid of organic or inorganic type, such as, but not limited to amorphous carbon, activated carbon, charcoal, silica gel, titania, circonia, or it may be a non-porous solid with high dispersity, i.e. of small particle size that provide for high surface to volume ratio, or it may be of mixed type, for instance, solid non-porous particles, which are covered with a layer of porous material.

The organic polymer, preferably cross-linked, can be a polystyrene polymer, or polyacrylate polymer, or polymethacrylate polymer, or polyurethane polymer, or polyamide polymer, or dextran polymer, such as, but not limited to Sephadex®, or agarose polymer, such as but not limited to Sepharose®, or a cellulose based polymer, or modified cellulose based polymer, such as but not limited to carboxymethylcellulose, or diethylaminoethyl cellulose, or methylcellulose, or other polysaccharide, or any other linear, branched, or cross-linked homo- or hetero-polymer or block copolymer, with iso- or atactic configuration, or with other tacticity, or may be any other appropriate macromolecule that is not soluble in the treated media.

For the treatment of aqueous based media, a hydrophilic organic polymer, or polymer which is wettable, or can expand, or swell in aqueous based media is highly preferred.

The reactive groups Q are preferably nucleophilic groups, such as, but not limited to thiosulfate, —OS(O)(O⁻)S⁻, or thiosufonate —S(O)(O⁻)S⁻, or mercapto or thiol groups, —SH, —CH₂SH, —CH₂CH₂SH, —CF₂CH₂SH, —OCH₂CH₂SH, —NH₂CH₂CH₂SH, —NH(Me)CH₂CH₂SH, —N(Me₂)CH₂CH₂SH, —COCH₂SH, —S(O₂)CH₂SH, thiourea, —NHC(S)NH₂, or substituted thiourea groups, thiocarboxylic acid, —C(O)S⁻, dithiocarboxylic acid, —C(S)S⁻, thiocarbonate O-esters, —OC(O)S⁻, dithiocarbonate O-esters, or xanthates, —OC(S)S⁻, thiophosphonate, —PO(OH)SH, and thiophosphate, —OPO(OH)SH, o-, m-, or p-thiophenyl group, —C₆H₄SH, thiosalicylate group, m-, or p-thiobenzoate group, —O₂CC₆H₄SH, or their salt forms.

In a preferred embodiment, Q is an —SH group which is directly connected to a double bond, or aromatic structure, or fully or partially sp² hybridized carbon atom.

In another preferred embodiment the —SH group has pKₐ of dissociation to —S⁻ and H⁺ that is less than 10, preferably less than 9, and most preferably less than 8.

In another embodiment the solid phase agent has the general structure XVIII:

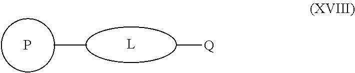

wherein:
P and Q are as in XVII, and L is a linker or a branched linker connecting the group Q with the solid phase agent matrix P, and where L can be linear of branched or dendrimeric and may contain one or more than one Q groups attached to it. Examples of L are divalent atom, or group of linearly connected atoms, which may be the same or different, and which are attached to the matrix P and to one of more groups Q, and may, or may not be connected to other atoms or groups of atoms. Particular examples of L can be oxygen, or sulfur atom, imino (NH) group, methylene, ethylene, propylene, ethoxyethylene groups, oligo- or polyoxiethylene, oligo- or polyester, or polyamide type linker. Especially preferred are polyethylene oxide type of linkers with length from 2 to 10000 monomer units, preferably from 8 to 200 monomer units.

In another embodiment, the solid phase agent contains not only nucleophilic groups Q, but also accessory groups K and it is depicted in general structures XIX and XX. The groups K do not react with, and covalently attach the compound(s) of Structure I. Instead, they assist the reaction of groups Q, with the compound(s) of Structure I.

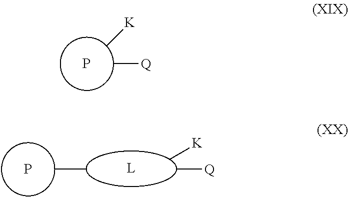

The function of the groups K can be, without being limited, to enhancing the nucleophilicity of the groups Q through the so-called neighboring effect, or neighboring electron pair effect, or by enhancing of the deprotonation of the nucleophilic groups Q, thus increasing of the number of the more nucleophilic anionic groups Q⁻, or by H-bonding to the nucleophilic groups Q, or by interacting with, and lowering of the energy of the transition state formed between compound(s) of Structure I and the nucleophilic group Q, or by non-covalent binding or ion-pairing with the compound(s) of Structure I thus increasing their local concentration, or by protonating, or complexing with the aziridine nitrogens of compound(s) of Structure I thus increasing their reactivity.

A reaction of an example of compound of Structure I with an example of a solid phase agent of structure XVIII is depicted below:

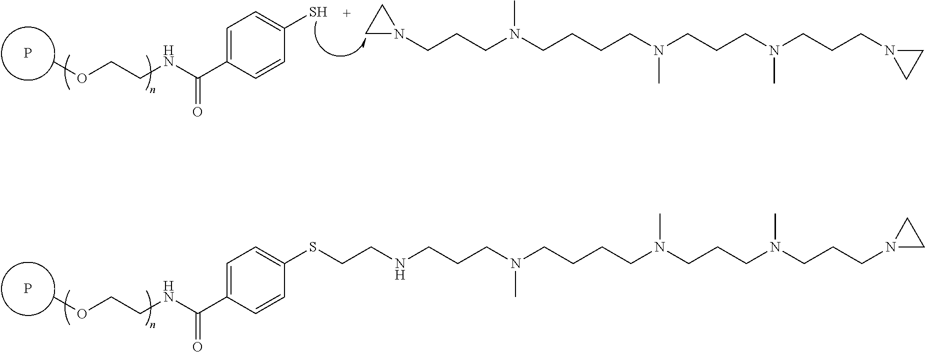

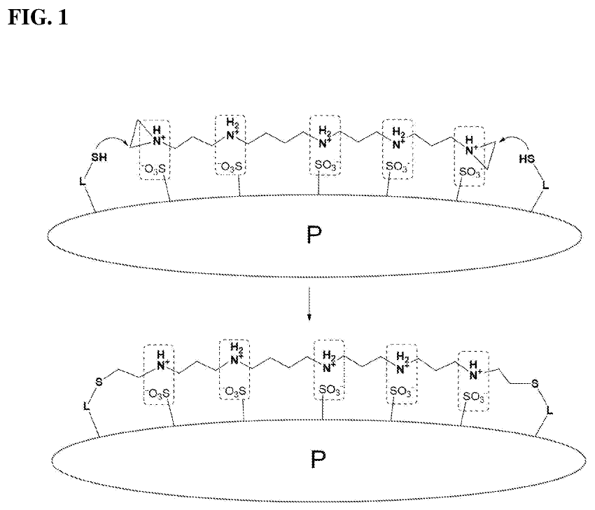

FIG. 1 illustrates the interaction of a representative compound of Structure I with a solid phase agent that has nucleophilic thiol groups attached through a linker L, and accessory anionic sulfo-groups directly attached to the polymer P matrix. The compound with Structure I is bound through multiple electrostatic interactions with the sulfo groups and is brought in the proximity of the nucleophilic SH groups, which attack the carbon atoms of the protonated, and therefore activated aziridine ring, opening it and covalently attaching the product of neutralization of compound of Structure I to the solid phase agent.

In another embodiment, the accessory groups K in structures XIX and XX is a hydrophilic group which has the function of enhancing the wettability or swelling of the matrix of the polymer P in aqueous environment. In many cases, the pathogen containing sample can have high aqueous content. Such examples are blood, blood products or components, other bodily fluids, interstitial fluid, cell growth culture or media, vaccine products or intermediates, or other biologics. Many polymers are of a hydrophobic nature, and therefore, without proper modification, may exclude aqueous-based fluids from their internal pore space, i.e., they are not wettable or cannot swell in such an environment, thus preventing the reactive groups Q from reacting with the compounds of Structure I. Introduction of sufficient number of hydrophilic accessory groups K can enhance the wettability of the interior of the porous solid phase agent, thus making reactive groups Q accessible for the aqueous solution containing compounds of Structure I. Examples of such hydrophilic groups can be, without being limited to, sulfo-, or sulphonyl groups, depicted in FIG. 1, or carboxylic groups, which have the additional advantage that they can bind through ion-pairing the polycationic compounds of Structure I. Other such hydrophilic groups can be hydroxy groups, or polyol groups, such as 2-hydroxyethyloxy (HOCH₂CH₂O), 2,3-dihydroxypropyloxy (HOCH₂CH(OH)CH₂O—), or oligo- and polyethylene glycol moieties with different number of monomer units.

Polymer matrix P of the solid phase agent, having Structures XVII to XX, can have an undesired effect on some components of some samples. For instance, the surfaces of many polymers, such as polystyrenes, polyurethanes, polymethacrylates, and polyamides can bind proteins from biologics, and biological fluids, or can disturb their conformation, structure and/or activity, activate the clotting cascade factors and the blood platelets, or elicit immune response. Modifying of such polymers by attachment of ethylene glycol oligomer or polymers of sufficient length and density can ameliorate or eliminate those problems. This approach, sometime referred by the skilled in the art as “pegylation” has been applied to many biopolymers, most often therapeutic proteins, as well as polymers which are in contact with biological fluids in vivo or in vitro as described by Harris M. J. (Ed.) in Poly(Ethylene Glycol) Chemistry. Biotechnical and Biomedical Applications, Plenum Press, New York and London, 1992 and references cited therein.

According to an embodiment of the present invention, the solid phase agent is divinylbenzene cross-linked polystyrene modified with nucleophilic reactive groups Q as described above and with polar groups which are ethylene glycol oligomers, or polyethylene glycols with molecular mass from 150 to 100,000 Da, preferably from 2,000 to 40,000 Da, and even more preferably from 4,000 to 20,000 Da and with density of up to one group at every monomer unit.

In another embodiment the polymer is acrylate or metacrylate polymer containing nucleophilic reactive groups Q and polar groups which are polyols, such as, but not limited to 2-hydroxyethyl, 2,3-dihydroxypropyl, di-, tri-, tetra-, penta-, or oligo-, or polyethylene glycol, and the polar groups are attached to the C-1, or the carbonyl group of the acrylate or metacrylate polymer in a density sufficient to achieve the desired hydrophilicity or other advantageous properties, which may be, without being limited to, lack of immunogenicity, or lack of thrombogenicity, or lack of binding or affinity to proteins, or receptors, or other components of the treated sample or composition or bodily fluids.

In another embodiment, the residual compound(s) with Structure I are removed by treatment of the sample with solid phase agent which has multiple anionic groups attached to it and binds the compounds with Structure I electrostatically through the formation of multiple ion-pair interactions with the positively charged nitrogen atoms of the compound(s) with Structure I. Such solid phase agents, and such approach, is disclosed herein for the removal of the products on neutralization of the compounds of Structure I. Since the compounds of Structure I are polycationic at close to neutral, neutral, or acidic pH, the same approach and solid phase agents can be used for the removal of the residual compounds of Structure I from the treated sample, media, composition, utility or organism.

In another embodiment of the invention, the residual compounds of Structure I are removed from the treated sample by contact with a solid phase agent which absorbs the compounds of Structure I. Such solid phase agent include, without being limited to, activated carbon, charcoal, amorphous carbon, amorphous silica, silica gel, amorphous alumina, titania or zirconia, or other solid phase agent which has absorbing affinity and capacity for the compounds of Structure I. The solid phase agent used for absorbing of the compound of Structure I preferably has high surface area to mass ratio, which may be achieved by using either a porous, micro-, or nano-porous solid, or highly dispersed non-porous solid. The porous absorbing solid phase agent may be shaped as powder, bulk solid, or particles of different size and shape, from micron size to 10 mm size. The preferred particle size is from 50 μm to 5 mm, and even more preferably from 0.1 mm to 0.5 mm, which particle size range provides for sufficiently sort diffusion time of the absorbed compounds to the bulk or the particle, and sufficiently high filtration or sedimentation rate of the particles for their removal.

In another embodiment, the absorbing solid phase agent may be brought in contact with the treated media, not directly, but though a semi-permeable barrier, which provides for the passage of the compounds that are intended to be absorbed, and does not allow the passage of components of the media, for which interaction with the solid phase agent is undesirable, as for examples proteins, or other macromolecules. Examples of such semi-permeable barrier are modified cellulose membranes or other dialysis membrane with molecular weight cutoff that allows for the diffusion of the compound(s) of Structure I prevents the diffusion of molecules with higher molecular weight, such as biopolymers.

In an embodiment of the method described herein, the method is used for inactivation of viruses, which may be enveloped, non-enveloped, DNA or RNA viruses, retro viruses, bacteriophages, or any other viruses. Examples of such viruses include, but is not limited to, hepatitis B (HBV), hepatitis C (HCV), human immunodeficiency virus (HIV; Types 1 and 2), malaria, syphilis, brucellosis, babesiosis, leptospirosis, arboviral infections (e.g., Colorado tick fever), relapsing fever, Chagas disease (Trypanosoma cruzi), West Nile virus (WNV), Human T-lymphotropic virus type I, and viral hemorrhagic fever (e.g., Ebola virus and Marburg virus).

In an embodiment of the method described herein, the method is used for the inactivation of prokaryotes such as archaea or bacteria, including Gram-positive and Gram-negative bacteria, spore forming bacteria and bacterial spores, or mycoplasma. Examples of pathogenic bacteria, and antimicrobial-resistant bacteria that can be treated with the methods provided herein include, without being limited to: Clostridium difficile (C. difficile), Enterobacteriaceae (CRE) bacteria, Neisseria gonorrhoeae, Campylobacter, Acinetobacter, Fluconazole-Resistant Candida, Extended Spectrum Enterobacteriaceae (ESBL), Tuberculosis (TB), Drug-Resistant Salmonella Serotype Typhi, Vancomycin-Resistant Enterococcus (VRE), Multi drug-Resistant Pseudomonas Aeruginosa, Drug-Resistant Non-Typhoidal Salmonella, Drug-Resistant Streptococcus Pneumoniae, Drug-Resistant Shigella, Methicillin-Resistant Staphylococcus Aureus (MRSA), Vancomycin-Resistant Staphylococcus Aureus, Erythromycin-Resistant Group A Streptococcus, Clindamycin-Resistant Group B Streptococcus, and others.

In another embodiment, the method is used for inactivation of eukaryote, single-, or multicellular eukaryote, including, but not limited to, fungi, protozoa, single- or multicellular parasite including helminths, schistosomes or nematodes or their eggs, single or multicellular algae and of crustacean.

The methods provided herein may be used for treatment of undesirable biological structures, including without limitation, of bacterial biofilms or other microorganism biofilms, lichens, encrustations or biofouling accumulations.

The method of the invention can be used to inactivate not only pathogenic microorganisms, but also non-pathogenic cells, such as leukocytes, when their presence in the treated sample is not desirable, as for instance in transfusable blood or blood products.

The methods provided herein may be used for inactivation of not only viruses, prokaryotes, and eukaryotes, but also for the inactivation of other infectious agents, such as prions, particularly when their pathogenic activity or infectivity is dependent on the presence or the activity of nucleic acids, in particular of ribonucleic acids as disclosed by Botsios, S. and Manuelidis, L. in “CID and Scrapie Require Agent-Associated Nucleic Acids for Infection”, J. Cell Biochem., 2016, 117, 1947-58 and by Supattapone, S. in “Synthesis of high titer infectious prions with cofactor molecules”, J. Biol. Chem., 2014, 289, 19850-4.

The methods provided herein may be used for the treatment of a sample, composition, media, utility or organism. The sample may be human or animal blood, leuko-depleted blood, whole blood, blood products, including plasma, serum, red blood cells or red blood cell concentrate, platelets or platelets concentrate, serum or plasma components, factors or enzymes, transfusion blood and blood components intended for transfusion, apheresis blood components, bodily fluids, animal sera, including sera used as cell culture additives, medium originated from eukaryotes or prokaryotes, vaccines, vaccine preparation compositions, suspension of microorganisms for preparation of whole pathogen killed vaccine; cosmetic and pharmaceutical compositions, beverage, food; or utilities, utensils, devices or their surfaces; or organisms, including animal, mammal or human organisms and parts thereof, including biological samples, and biopsies. The method can be used for treatment of biologics, including but not limited to, antibodies, immunoglobulins, hormones, enzymes, growth factors, coagulation factors, albumins or complement system components. The utilities can be, without limitation, medical or veterinary devices, including disposable devices, and instruments. The utility includes, without limitation, industrial or household equipment, appliances, apparatuses, mechanisms, machinery, or materials, or any other articles where pathogens or other organisms' presence may be undesirable or need to be controlled. The utility also includes without limitation, pipe, duct, hose, pipeline, vent, heat exchanger, sewer, channel, or any other fluid or gas conduit, or any surface which is in contact with aqueous fluid, such as sea vessels, screens, or filters, where pathogens, microorganisms, or other organisms' presence is undesirable or in need of control, as for example in biofouling.

The method for pathogen inactivation may be performed in transfusion blood or blood products, in which the treatment with the compound(s) of Structure I and the following treatment for their removal, inactivation, and products or inactivation and/or inactivators' removal is done in a sterile, partially, or fully closed system.

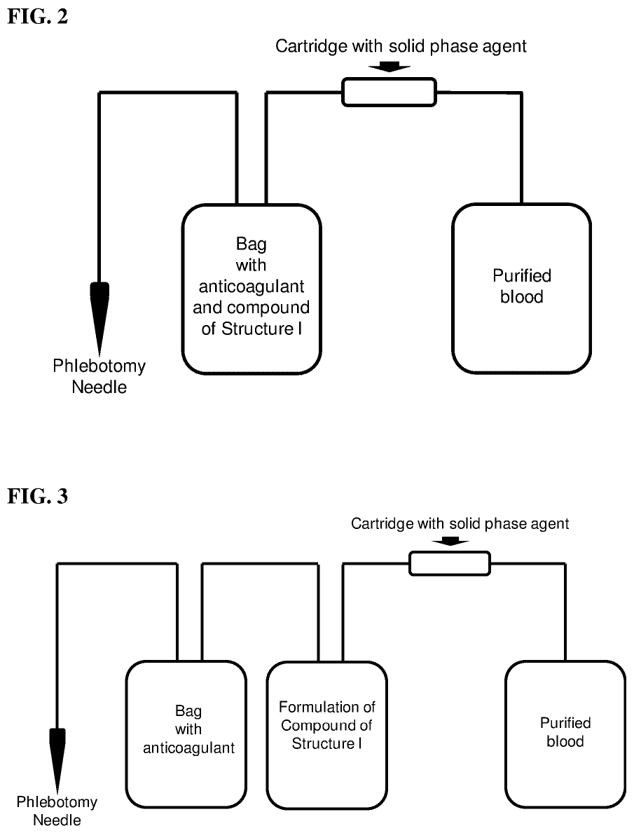

In some embodiments, the compound of Structure I is loaded in a blood collection bag together with the anticoagulant solution as illustrated in FIG. 2.

In other embodiments, the compound of Structure I formulated as liquid or solid formulation is loaded in a separated blood bag, as illustrated in FIG. 3.

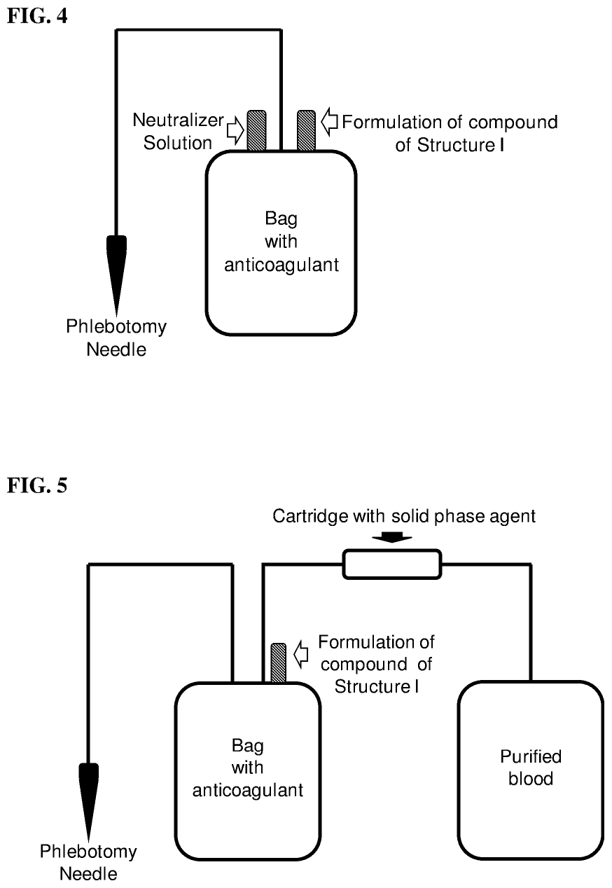

In other embodiments, the compound of Structure I, formulated as liquid or solid formulation is pre-loaded in a small container, which is attached to the blood collection or blood treatment bag and separated from it by a breakable seal as illustrated in FIGS. 4 to 9.

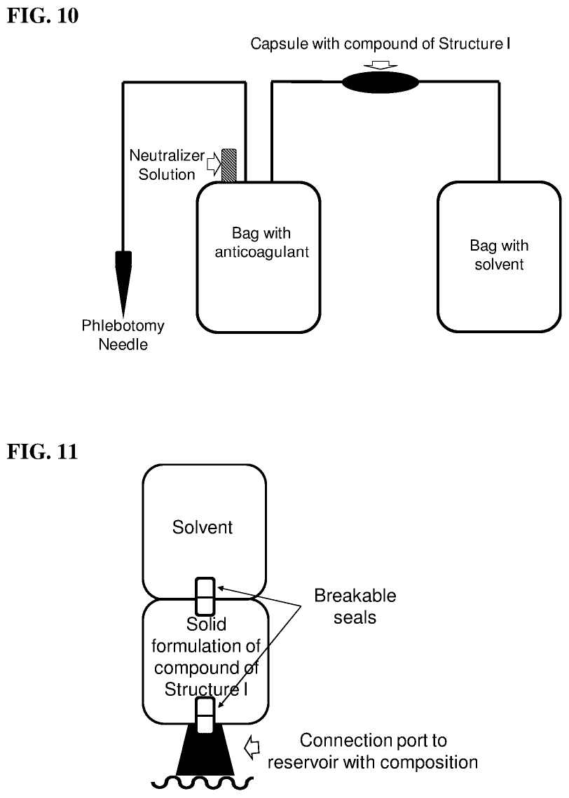

In other embodiments, the compound of Structure I is loaded in a capsule, which is connected through a breakable seal to a container with solution and with another breakable seal to the blood treatment bag as illustrated in FIG. 10.

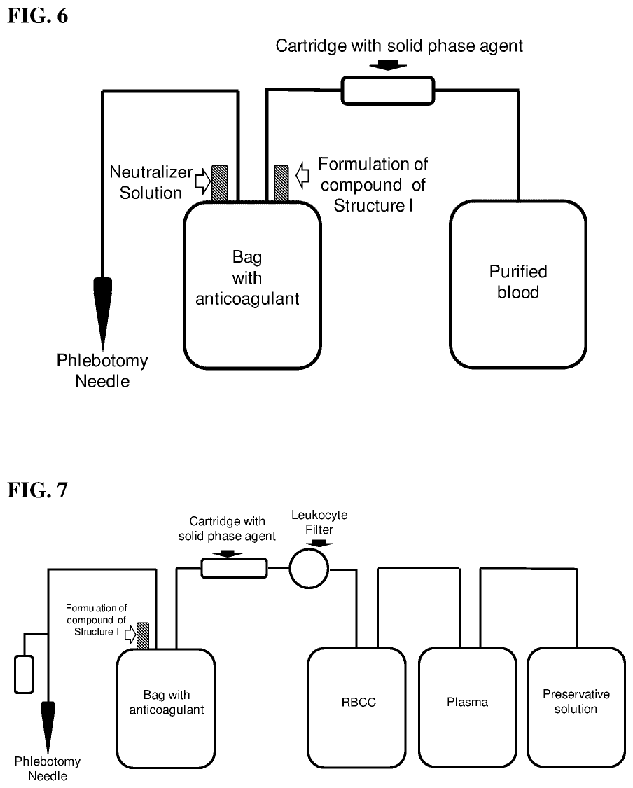

In some embodiments, the solution or liquid formulation of the neutralizer is placed in a container, which is attached to the blood treatment bag through a breakable seal, as illustrated in FIGS. 4 and 6, or can be placed directly in a neutralization treatment blood bag. The solid phase agent for removal of the residual compound of Structure I or the products of its neutralization or of the neutralizators can be placed in a cartridge, wherein the cartridge is connected through breakable seals to a treatment and to a receiving bag, as illustrated in FIGS. 2, 3, 5, 6, 7 and 8 or can be placed in a blood bag in form of free beads, or in semi-permeable container (pouch), as illustrated in FIG. 9.

The method for using the whole blood unit closed processing system illustrated in FIG. 2 is: Step 1—collection of blood by phlebotomy needle in collection bag containing anticoagulant and compound of Structure I; Step 2—Incubation for pathogens inactivation; Step 3—removal of the residual compound of Structure I by passing of the treated blood through a cartridge containing a solid phase agent and collection of the purified blood in the purified blood bag.

The method for using the whole blood unit closed processing system illustrated in FIG. 3 is: Step 1—collection of blood by phlebotomy needle in a collection bag containing anticoagulant; Step 2—Transfer of the anticoagulated whole blood in the treatment bag containing the solid formulation of the compound of Structure I, mixing and incubation for pathogens inactivation; Step 3—removal of the residual compound of Structure I by passing of the treated blood through a cartridge containing a solid phase agent and collection of the purified blood in the purified blood bag.

The method for using the whole blood unit processing closed system illustrated in FIG. 4 is: Step 1—collection of blood by phlebotomy needle in a bag containing anticoagulant; Step 2—unsealing of a capsule containing liquid formulation of the compound of structure I and adding the formulation to the blood; Step 3—incubation of the blood with the compound of Structure I; Step 4—breaking of the capsule and addition of the liquid formulation of the inactivators, mixing and incubation for neutralization of the compound of Structure I.

The method for using the whole blood unit closed processing system illustrated in FIG. 5 is: Step 1—collection of blood by phlebotomy needle in collection bag containing anticoagulant; Step 2—unsealing of a capsule containing liquid formulation of the compound of Structure I and adding the formulation to the blood; Step 3—mixing and incubation of the blood with the compound of Structure I; Step 4—removal of the residual compound of Structure I by passing treated blood through a cartridge containing a solid phase agent and collection of the purified blood in the purified blood bag.

The method for using the whole blood unit processing closed system illustrated in FIG. 6 is: Step 1—collection of blood by phlebotomy needle in a bag containing anticoagulant; Step 2—unsealing of a capsule containing liquid formulation of the compound of structure I and adding the formulation to the blood; Step 3—incubation of the blood with the compound of Structure I; Step 4—breaking of the capsule and addition of the liquid formulation of the inactivators, mixing and incubation for neutralization of the compound of Structure I; Step 5—removal of the products of neutralization of the compound of Structure I by passing treated blood through a cartridge containing the solid phase agent.

The method for using of the whole blood unit processing closed system illustrated in FIG. 7 is: Step 1—collection of blood by phlebotomy needle in a bag containing anticoagulant; Step 2—unsealing of a capsule containing liquid formulation of the compound of Structure I and adding the formulation to the blood; Step 3—incubation the blood with the compound of Structure I; Step 4—removal of the residual compound of Structure I and leukofiltration by passing of the treated blood through a cartridge containing the solid phase agent and a leukofilter; Step 5—centrifugation of the purified leukodepleated blood in the RBCC bag; Step 6—transferring of the separated plasma to the plasma bag; Step 7—transferring of the preservative solution to the red blood cells and mixing to prepare blood cells concentrate.

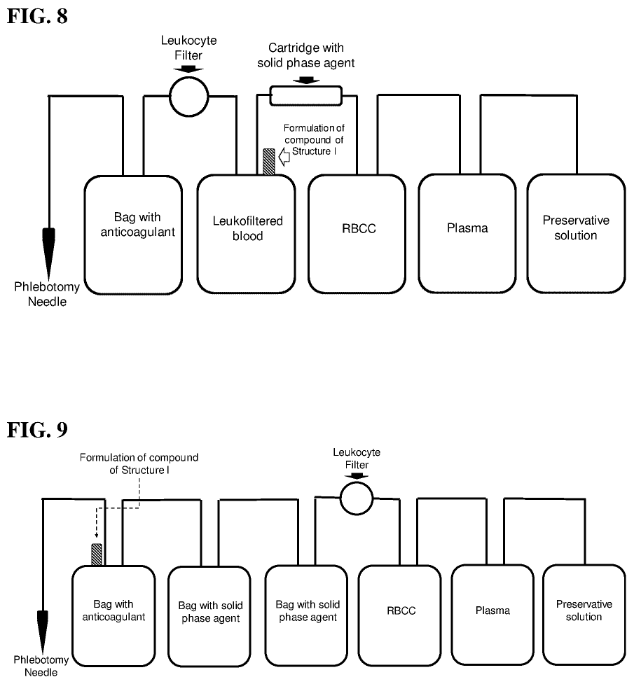

The method for using the whole blood unit processing closed system illustrated in FIG. 8 is: Step 1—collection of blood by phlebotomy needle in a bag containing anticoagulant; Step 2—leukodepletion of the whole blood by filtering through a leukofilter into LF blood bag; Step 3—unsealing of a capsule containing liquid formulation of the compound of Structure I and adding the formulation to the leukofiltered blood in LF blood bag; Step 4—mixing and incubation the blood with the compound of Structure I; Step 5—removal of the residual compound of Structure I and by passing of the treated blood through a cartridge containing a solid phase agent; Step 6—centrifugation of the purified leukodepleated blood in the RBCC bag; Step 7—transferring of the separated plasma to the plasma bag; Step 8—transferring of the preservative solution to the red blood cells and mixing to prepare blood cells concentrate.

In some embodiments of the invention, reduction of the residual compound of Structure I to the desired level by a single treatment with a solid phase agent may not be achieved. In such cases, two or more subsequent treatments with the solid phase agent may be required, as it is illustrated in FIG. 9.

The method for using of the whole blood unit processing closed system illustrated in FIG. 9 is: Step 1—collection of blood by phlebotomy needle in a bag containing anticoagulant; Step 2—unsealing of a capsule containing liquid formulation of the compound of Structure I and adding the formulation to the blood; Step 3—mixing and incubation of the blood with the compound of Structure I; Step 4—removal of the residual compound of Structure I by transferring of the treated blood to the first bag with solid phase agent (either as free flowing beads, or packed in semi-permeable pouch) and incubation; Step 5—second removal of the residual compound of Structure I after the first removal step by transferring of the blood to the second bag with solid phase agent (either as free flowing beads, or packed in semi-permeable pouch) and incubation; Step 6—leukofiltration by passing of the treated blood through a leukofilter to the RBCC bag; Step 7—centrifugation of the purified leukodepleated blood in the RBCC bag; Step 8—transferring of the separated plasma to the plasma bag; Step 9—transferring of the preservative solution to the red blood cells and mixing to prepare blood cells concentrate.

The method for using of the whole blood unit processing system illustrated in FIG. 10 is: Step 1—collection of blood by phlebotomy needle in a bag containing anticoagulant; Step 2—unsealing of a capsule containing formulation of the compound of Structure I and dissolving the compound of Structure I in solvent from solvent bag; Step 3—addition of the solution of the compound of Structure I in the collected blood, mixing and incubation; Step 4—Addition of the neutralizer solution and incubation to neutralize the residual compound of Structure I.

Another example of a container using a solid formulation of a compound of Structure I connected through a breakable seal to a container of the solvent for dissolving of the formulation and through another breakable seal to a container with the sample to be treated is illustrated in FIG. 11.

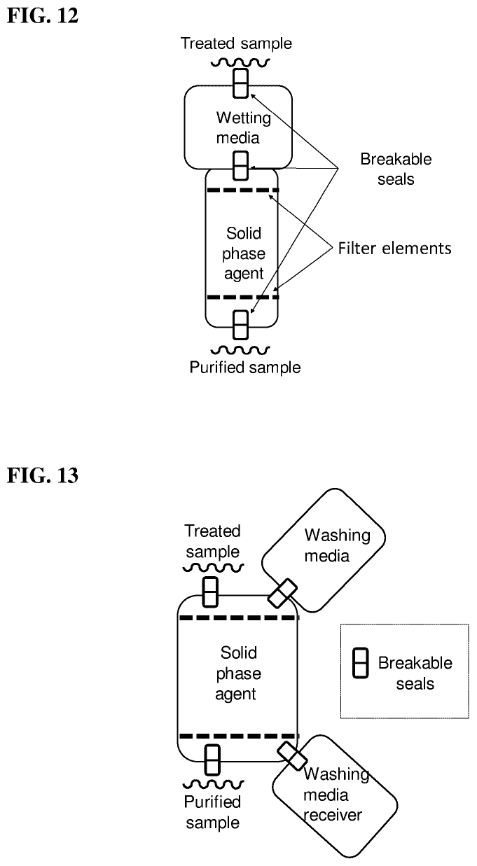

In another embodiment, the solid phase agent is packed in a cartridge and stored in said cartridge in dry form and is pre-wetted and/or rinsed prior use by liquid composition compatible with the treated sample and its method of use. As for an example, FIG. 12 illustrates a closed system comprising a cartridge packed with dry solid phase agent that is contained between two filtering elements. The cartridge is connected through breakable seals to a container containing the wetting media and through another breakable seal to the container for purified sample. The wetting media container is connected through a breakable seal to a container for treatment of the sample with compound(s) of Structure I. Breaking of the seal between the cartridge and the container with the wetting media and transferring of the media in the cartridge provides for the solid phase agent wetting. Breaking of the remaining seals allows for the passage of the treated sample through the wetted solid phase agent.

In another embodiment, the solid phase agent is rinsed under sterile conditions before use. Such rinsing may be important to minimize or eliminate leachables that may accumulate in the solid phase agent during the storage period. The washing is done preferably with a composition that is compatible with the solid phase agent, the treated sample and its intended use. FIG. 13 illustrates a closed system where the solid phase agent, packed in a cartridge, is rinsed by solvent contained in a container connected to the solid phase agent cartridge through a breakable seal. The washing media is then collected in another integrated container after breaking the seal between the cartridge and the container. The two breakable seals are then re-sealed by appropriate clips or resealing devices such as T-Seal (Terumo tube sealing device). Breaking of the remaining seals allows for the passage of the treated sample through the washed solid phase agent.

In some embodiments, the solid phase agent is contained in the cartridge/columns between permeable barriers on both or on one end of the cartridge/column. The barriers allow for the passing of the treated sample through the cartridge, but do not allow for the passing of the solid phase agent. Examples of such barriers are, without limitation, filters/screens, disks made of sintered material, mesh, sieve or textile, or any other porous material, or material with opening or channels with a size smaller than the size of the solid phase agent particles. Such barriers are indicated in FIGS. 12 and 13 with interrupted lines.

In another embodiment, the disclosed closed system for pathogen inactivation according to the method is sterilized by UV or gamma irradiation, thermal treatment, high or low pH solvent treatment, or other chemical treatment, such as with ethylene oxide, ozone, bleach, glutaraldehyde, formaldehyde, hydrogen peroxide, peracetic acid or silver compounds, or by other methods known to one skilled in the art. The liquid formulation of the compounds of Structure I and their neutralizers may be sterilized by filtration, UV or gamma irradiation, thermal treatment, or other methods known to one skilled in the art. The solid phase agent may be sterilized by UV or gamma irradiation, thermal treatment, high or low pH solvent treatment, chemical treatment, either before or after packing in a cartridge or other container or semi-permeable pouch, and either before or after integration in the closed system.

The examples of pathogen reduction closed systems in FIGS. 2 to 13 are provided for illustrative purposes and are not intended to limit the scope of the invention.

In some embodiments, the pathogen(s) are present in an organism, which organism may be an animal, a mammal, including primate, rodent, sea mammal, or any wild or domesticated animal or a human. In these embodiments, the treatment with compounds of Structure I is done in vivo. This in vivo treatment is done by intravenous, oral, topical, rectal, subcutaneous, intramuscular administration, by inhalation, or by combination thereof, and the treatment can be done by a single administration, by multiple administrations, or by continuous administration and at dose(s) sufficient to achieve the desired pathogen's reduction. Such in vivo treatment may be followed or combined with in vivo treatment with an inactivator of the compound of Structure I such as, but not limited to sodium thiosulfate.

In other embodiments, the treatment of the organism with compound of Structure I is done in vivo; and the neutralization/and or removal of the compound(s) of Structure I or the removal of the products of their neutralization or degradation is done ex vivo, by treatment of bodily fluids of the organism, such as blood or plasma, followed by their return (transfusion) back to the organism. Such ex vivo treatment may be done in batch, by periodical removal of portion of a bodily fluid, treatment, and transfusion, or by continuous withdrawal, treatment and transfusion. It this later case, the use of an apheresis process, and continued treatment of apheresis plasma is preferred. The neutralization or removal of the compounds of Structure I may be done by passing through a cartridge containing a solid phase agent which sequesters the compound(s), or by mixing with a solution of a neutralizing agent, followed by incubation, which may be followed by passing through a cartridge with a solid phase agent for sequestering of the products of neutralization and/or the neutralizing agent.

In other embodiments, the treatment of the pathogen-containing organism is done by ex vivo treatment of said organism's bodily fluids. This treatment may be done in batch, by periodical removal of portion of a bodily fluid, treatment, and transfusion; or by continuous withdrawal, treatment and transfusion. It this later case the use of an apheresis process and continued treatment of apheresis plasma is preferred. The ex vivo treatment is done by adding of appropriate amount of formulation of compound(s) of Structure I to the bodily fluid and incubation, which may be followed, preferably, by treatment for removal or neutralization of the residual compound(s) of Structure I and/or, optionally, by treatment for removal of the products of inactivation or degradation of the compounds of Structure I, followed by transfusion of the purified bodily fluid back to the organism. The treatment for removal or neutralization and/or removal of the products of neutralization of the compounds(s) of Structure I is done as described above for in vivo treatment with compound(s) of Structure I.

In a preferred embodiment of the method for in vivo or ex vivo treatment of organism with compound(s) of Structure I at least one of the pathogens, present in the organism, and targeted for inactivation by the treatment is resistant to one or more antipathogen treatments.

### EXAMPLES

### Example 1

### Synthesis of the compound VI, N¹,N⁴-bis(3-(aziridin-1-yl)propyl)-N¹,N⁴-dimethylbutane-1,4-diamine

A. Synthesis of aziridine: 2-Chloroethylamine hydrochloride, 58.4 g (0.503 mol) was dissolved in 100 ml water. The solution was added dropwise with stirring to a solution of 56.4 g sodium hydroxide in 20 mL of water. After additional stirring for 2.5 h at 50° C. aziridine was purified by distillation under partial vacuum. Solid NaOH was added in portions to the distillate under vigorous stirring and cooling at temperature 0-8° C. The mixture was stirred at this temperature for 30 min. The liquid was decanted from the solid NaOH, and the top layer was separated to give 22.5 g of wet aziridine. This material was dried by addition of small portions of powdered KOH and decanting after each portion, until KOH retained dry appearance. The resulted dry aziridine stored under KOH pallets at −20° C. Yield, 16.02 g, 74% of clear liquid.

B. Synthesis of 2-(1-aziridinyl)propanal mono-methyl acetal, IV: Acrolein, 6.65 g, 7.93 ml, 0.120 mol was added to 100 ml MeOH. The solution was flushed with Ar. and cooled under Ar in dry ice bath. Aziridine, 4.99 g, 6.00 ml, 0.124 mol was added dropwise and on stirring. The dry ice bath was removed, and the reaction mixture was left to room temperature. Thus obtained solution of 2-(1-aziridinyl)propanal mono-methyl acetal, IV was stored sealed under Ar and at −20° C. ¹H NMR (300 MHz, CD₃OD) δ: 4.66 (t, J=5.54 Hz, 1H), 3.36 (s, 3H), 2.30-2.44 (m, 2H), 1.79-1.93 (m, 2H), 1.76-1.79 (m, 2H), 1.30-1.33 (m, 2H). ¹³C NMR (75 MHz, CD₃OD) δ: 97.9, 57.5, 36.5, 26.6.

C. Synthesis of N¹,N⁴-bis(3-(aziridin-1-yl)propyl)-N1,N4-dimethylbutane-1,4-diamine, VI: The methanol solution of compound IV from step B was cooled in ice bath. N,N′-Dimethylbutane-1,4-diamine, 5.85 g, 50.4 mmol was added dropwise and on stirring. The bath was removed, and after 30 min sodium borohydride, 10 g was added on portions on stirring and cooling at −4-+4° C. After 4 hours at rt and aqueous work up and extraction with ether the product was purified by silica gel chromatography. The fractions containing the product were evaporated and the residue was subjected to vacuum distillation to give 3.84 g compound VI as a light-yellow oil. ¹H NMR (300 MHz, C₆D₆) δ: 2.43 (t, J=7.2 Hz, 4H), 2.30 (m, 4H), 2.13 (t+s, J=6.7 Hz, 10H), 1.75 (m, 4H), 1.55 (m, 4H), 1.51 (m, 4H), 0.79 (m, 4H). ¹³C NMR (75 MHz, C₆D₆) δ: 60.74, 58.55, 56.49, 42.52, 28.77, 27.50, 26.11. MS (Electrospray, positive mode) m/z: 283.1, calc. [M+H]⁺ 283.2.

### Example 2

### Synthesis of Compound XVI, 3-(aziridin-1-yl)-N-(3-(aziridin-1-yl)propyl)-N-methylpropan-1-amine

Compound XVI was synthesized as in Example 1, using 3.91 g, 4.35 ml 40% solution of methylamine in water instead of N,N′-dimethylputrescine. After fractional vacuum distillation 2.48 g of compound XVI were obtained as light oil. ¹H NMR (500 MHz, C₆D₆) δ: 2.43 (t, J=7.0 Hz, 4H), 2.12 (s, 3H), 2.11 (t, J=7.0 Hz, 4H), 1.74 (m, 4H), 1.54 (m, 4H), 1.51 (m, 4H), 0.77 (m, 4H). ¹³C NMR (75 MHz, C₆D₆) δ: 60.00, 55.72, 41.78, 28.01, 27.50, 26.79. MS (Electrospray, positive mode) m/z: 198.1, calc. [M+H]⁺ 189.2.

### Example 3

### Synthesis of Compound X, N¹-(3-(aziridin-1-yl)propyl)-N⁴-(3-03-aziridin-1-yl)propyl)(methyl)amino)-propy 1)—N¹,N⁴-dimethylbutane-1,4-diamine

A. Synthesis of N¹,N⁵,N¹⁰-trimethylspermidine: Spermidine, 5.70 g, 6.16 ml, 39.3 mmol was mixed with ethyl formate, 61.1 g, 66.6 ml, 0.824 mol, and the mixture was refluxed for 30 h, and then evaporated under vacuum to give N¹,N⁵,N¹⁰-triformylspermidine, 9.32 g, as oil. Lithium aluminium hydride, 9.00 g was added to dry tetrahydrofuran, 300 ml. N¹,N⁵,N¹⁰-Triformyl spermidine, 9.00 g was added dropwise under Ar and on stirring. The reaction mixture was refluxed for 4 h, and then cooled to rt. Water, 22 ml was added dropwise on cooling and efficient mechanical stirring (frothing), followed by 90 ml 50% potassium hydroxide solution in water. After vigorous stirring for 1 h, tetrahydrofuran, 150 ml was added and the layers were separated. The bottom layer was extracted with 150 ml tetrahydrofuran, and the extract was combined with the top layer. The combined organic layers were evaporated under vacuum, and the residue was dissolved in diethyl ether, 75 ml and dried overnight over solid potassium hydroxide. The dry ether solution was evaporated and the residue was subjected to fractional vacuum distillation to give 5.30 g of N¹,N⁵,N¹⁰-trimethylspermidine. H NMR (300 MHz, C₆D₆) δ: 2.53 (t, J=6.7 Hz, 2H), 2.45 (t, J=6.6, 2H), 2.22-2.35 (m, 4H), 2.30 (s, 3H), 2.28 (s, 3H), 2.12 (s, 3H), 1.58 (m, 2H), 1.46 (m, 4H). ¹³C NMR (75 MHz, C₆D₆) δ: 58.28, 56.52, 52.44, 51.03, 42.21, 36.83, 28.21, 28.19, 25.67. MS (Electrospray, positive mode) m/z: 188.1, calc. [M+H]⁺ 188.2.

B. Synthesis of Compound X: Compound X was synthesized as per Example 1, using 3.71 g, 4.43 ml, 67 mmol acrolein; 56 ml methanol; 2.79 g, 3.35 ml, 69 mmol aziridine; 5.30 g, 28.1 mmol of N¹,N⁵,N¹⁰-trimethylspermidine instead of N,N′-dimethylputrescine, and 5.58 g sodium borohydride. After work up and fractional vacuum distillation, 2.99 g of compound X were obtained as off-white oil. ¹H NMR (300 MHz, C₆D₆) δ: 2.39-2.45 (m, 4H), 2.32-2.38 (m, 4H), 2.27-2.31 (m, 4H), 2.14 (s, 6H), 2.13 (s, 3H), 2.10-2.15 (m, 4H), 1.73 (quintet, J=7.0 Hz, 4H), 1.58-1.68 (m, 2H), 1.54-1.56 (m, 4H), 1.47-1.53 (m, 4H), 0.80-0.82 (m, 4H). ¹³C NMR (75 MHz, C₆D₆) δ: 60.74, 58.59, 58.55, 56.60, 56.56, 56.51, 56.48, 42.65, 42.54, 28.76, 27.50, 26.43, 26.14, 26.10. MS (Electrospray, positive mode) m/z: 354.1, calc. [M+H]⁺ 354.3.

### Example 4

### Synthesis of the Compound XIV, N¹,N⁴-di(3-((3-(aziridin-1-yl)propyl)-(methyl)amino)propyl)-N¹,N⁴-dimethylbutane-1,4-diamine

A. Synthesis of N¹,N⁵,N¹⁰,N⁴-tetramethylspermine: N¹,N⁵,N¹⁰,N¹⁴-tetramethylspermine was prepared as per Example 3 A, from spermine, 1.60 g, 7.86 mmol, through tetraformylspermine, followed by reduction with lithium aluminium hydride, 2.00 g in 50 ml dry tetrahydrofuran, and was isolated after aqueous work up and fractional vacuum distillation as 1.59 g of off-white oil. H NMR (300 MHz, C₆D₆) δ: 2.53 (t, J=6.7 Hz, 4H), 2.34 (t, J=6.9, 4H), 3.30 (s, 6H), 2.28 (m, 4H), 2.13 (s, 6H), 1.59 (quintet, J=6.8 Hz, 4H), 1.50 (m, 4H), 0.87 (bs, 2H). ¹³C NMR (75 MHz, C₆D₆) δ: 57.92, 56.25, 50.75, 41.94, 36.53, 27.88, 25.37. MS (Electrospray, positive mode) m/z: 258.1, calc. [M+H]⁺ 258.3.

B. Synthesis of Compound XIV: Compound XIV was synthesized as per Example 1, using 10 mmol of 3-(aziridin-1-yl)propanal in 9 ml methanol; 0.80 g, 3.1 mmol of N¹,N⁵,N¹⁰,N¹⁴-tetramethylspermine instead of N,N′-dimethylputrescine, and 0.77 g sodium borohydride. After aqueous work up, fractional vacuum distillation, and silica gel chromatographic purification, 0.398 g of compound XIV were obtained as off-white oil. ¹H NMR (300 MHz, C₆D₆) δ: 2.44 (t, J=7.0 Hz, 4H), 2.34-2.40 (m, 8H), 2.31 (m, 4H), 2.15 (s, 6H), 2.14 (s, 6H), 2.11-2.16 (m, 4H), 1.75 (quintet, J=7.0 Hz, 4H), 1.65 (quintet, J=7.4 Hz, 4H), 1.53 (m, 4H), 1.47-1.53 (m, 4H), 0.79-0.81 (m, 4H). ¹³C NMR (75 MHz, C₆D₆) δ: 60.77, 58.64, 56.64, 56.60, 56.54, 42.65, 28.78, 27.51, 26.46, 26.18. MS (Electrospray, positive mode) m/z: 425.2, calc. [M+H]⁺ 425.4.

### Example 5

### Reactivity Toward Nucleic Acids

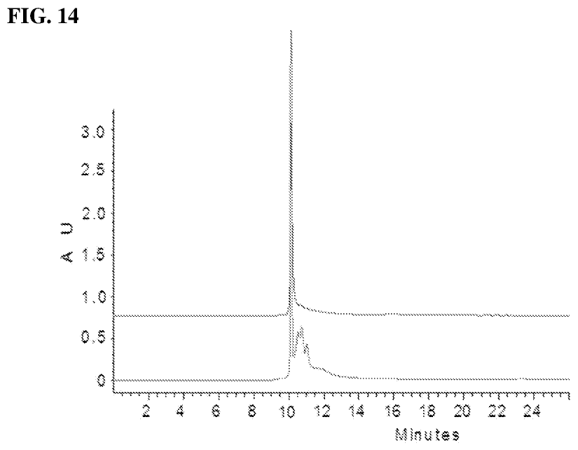

Reactivity toward nucleic acids was followed by the reaction of 10 μM 21-mer synthetic oligodeoxyribonucleotide—5′ ATA CCT CAT GGT AAT CCT GTT—3′ (SEQ ID NO:1), comprising all four nucleobases in its sequence with 200 μM Compound X in PBS (pH 6.7) at 37° C. FIG. 14 illustrates the HPLC analysis of the incubation mixture after 0 h (top chromatogram) and 6 h (bottom chromatogram) incubation at 37° C. The peak corresponding to the oligonucleotide diminishes and compounds with higher retention time appear, clearly demonstrating the appearance of covalent adducts of Compound X with the oligonucleotide.

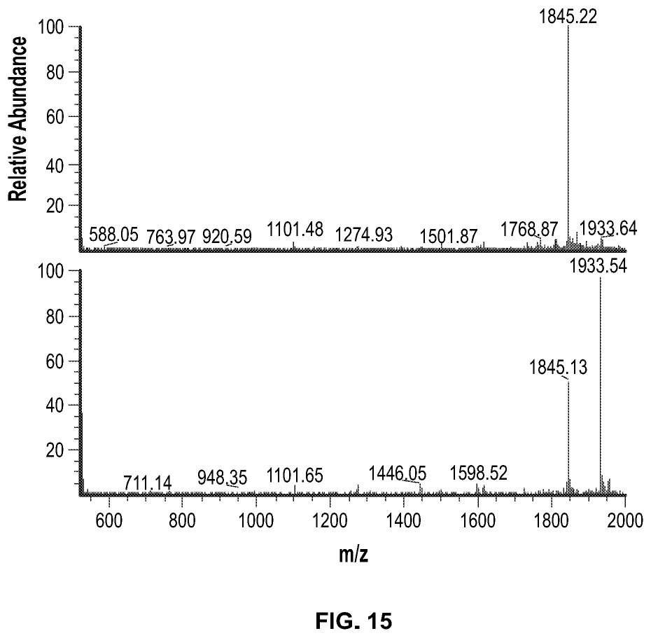

The reaction of a synthetic 23-mer oligoribonucleotide (UGG ACU CCG AUA ACG GAG UAU GU (SEQ ID NO:2)), 100 μM with Compound X, 100 μM in PBS at pH 7 and at room temperature was studied by mass spectrometry. The results are shown in FIG. 15, where the top panel is the mass spectrum of the oligonucleotide before the treatment, and in the bottom panel is the mass spectrum of the reaction 6 min after the addition of compound X. The 1845.22 m/z peak in the top panel is due to the oligonucleotide ion with a charge state of minus 4, (M−4H)/4 and corresponds to a neutral molecule with mass of 7384.9 Da (calculated oligonucleotide mass, 7384.0 Da). In the bottom spectrum, an additional peak appears after 6 min incubation with compound X, with m/z of 1933.54 corresponding to the neutral molecule with mass of 7738.2 Da. The molecular mass of the covalent mono-adduct of Compound X with the oligonucleotide is 7737.3 Da.

### Example 6

Lack of Reactivity of Compounds of Structure I with Cytochrome C

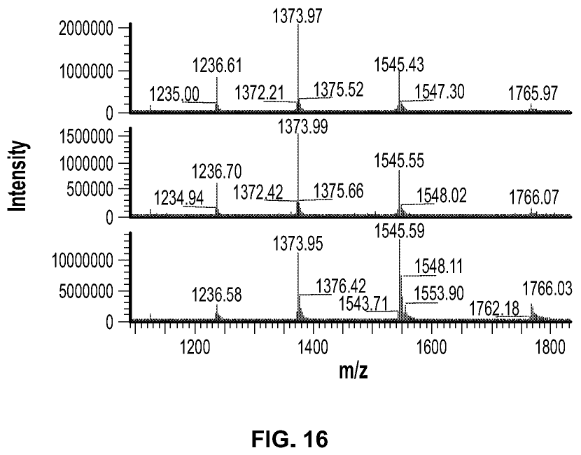

Using alkylating molecules to inactivate pathogens in blood product has potentially harmful side effect—their reaction with proteins may create neoantigens, i.e., they may become haptens. To assess the hapten potential of the compounds of Structure I, their ability to modify Cytochrome C was studied. Cytochrome C (MW of 12384 Da) was selected as a model protein because it contains a number of amino acids with nucleophilic side chains: 19 Lys, 2 Cys, 3 Asp, 9 Glu, 3 His, and 4 Tyr, which are potential targets for alkylation by the compounds of Structure I. Cytochrome C, 0.1 mg/mL (8 μM) solution in phosphate buffered saline was incubated with 0 (control), 0.1, 1, and 10 mM of compounds VI or X at pH 7.0 for 30 h at 40° C. Aliquots of the incubation mixtures were analyzed at 1, 4 and 30 h by electrospray mass spectrometry in the positive ionization mode with direct infusion into a LCQ Advantage mass spectrometer (Thermo-Finnigan, San Jose, CA) for the formation of covalent adducts of the protein with the test compounds. The results show unambiguously the absence of covalent adducts of both test compounds VI and X with cytochrome C at any of the concentrations and time points (see FIG. 16 for representative mass spectra).

### Example 7

Lack of Reactivity of the Compounds of Structure I with Virus Surface Proteins

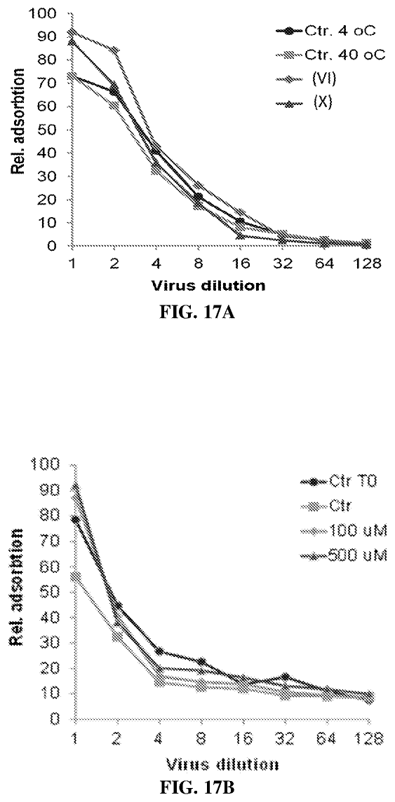

The potential of the compounds of Structure I to modify pathogens' proteins was evaluated using respiratory syncytial virus (RSV) as a model pathogen and RSV's fusion (F) was selected for testing for modifications. The F protein is a large (574 amino acids) viral envelope-associated surface glycoprotein, which plays an important role in host recognition and virus insertion. This protein was selected because of its high sensitivity and instability, and the availability of monoclonal antibodies specific to different antigenic epitopes and sensitivity to F protein conformational changes. Sucrose gradient-purified RSV was treated with compound VI and compound X, both at 100 μM concentration for 4 hours at 40° C. The residual compounds VI and X were neutralized as described in Example 16. Controls included mock-treated RSV incubated for 4 hours at 40° C. and non-treated virus kept at 4° C. ELISA assay was performed according procedure described by Schmidt et al, J Virol. 2014; 88(17):10165-76. doi: 10.1128/W1.01250-14. PubMed PMID: 24965456. Eight serial 1:2 dilutions in PBS were plated (50 μl/well) in triplicates into 96-well plates, and incubated overnight at 4° C. The wells were washed with PBS and blocked with PBS/1% BSA. Anti-protein F antibody was added, and the mixture was incubated for 2 h, followed by washings and the addition of anti-mouse IgG HRP conjugate. After another round of washing, TMB substrate and sulfuric acid were added and readings were conducted using ELISA Reader SPECTRAmax PLUS (Molecular Devices, Sunnyvale, CA). In FIGS. 17A and 17B, the results of anti-F antibody binding to compounds VI and X-treated RSV determined by ELISA are presented. From these experiments, it was clear that treatment with compounds VI and X under conditions which completely inactivated the virus, did not change the degree of recognition of the F protein by a highly specific, conformationally sensitive monoclonal antibody, indicating that no modification to the F protein by the treatment occurred.

### Example 8

### Bacterial Inactivation by Compound VI, Compound X, and Compound XIV in Bacteria Growth Medium

A panel of G+F and G− bacteria were inactivated in their respective growth medium using compound VI, compound X, and compound XIV. All cells were grown in corresponding media to middle log phase, collected by centrifugation, re-suspended in Ringer's solution (RS), and treated at RT with 100 μM of compound VI, compound X, and compound XIV, which were added to the suspension as 100× concentrate in RS. Controls received RS only. At the end of the incubation, unreacted compound VI, compound X, and compound XIV were neutralized with 10 mM sodium thiophosphate during incubation at RT for 30 min. The viable cells were enumerated using serial dilutions by standard colony agar plate count. Table 1 summarizes the typical results of 1 h treatment of E. coli, P. fluorescens, Y. enterocolitica, B. cereus, S. aureus, and S. epidermidis with compound VI, compound X, and compound XIV. Clearly, even after 1 h treatment, the reduction in viable cells was observed for all three compounds. Compound X and compound XIV showed significantly higher potency than compound VI. With these two compounds, two species were inactivated to below the limit of detection (1.00 Log₁₀ CFU/mL).

TABLE 1
Bacterial inactivation at RT with 100 μM compounds VI, X, and XIV

| | Gram | Titer (Log₁₀CFU/mL) | | | | |
|---|---|---|---|---|---|---|
| Species | Type | Initial | Contr. | Cmpd. VI | Cmpd. X | Cmpd. XIV |
| E. coli | − | 8.08 | 8.11 | 4.60 | 3.98 | 3.51 |
| P. fluorescens | − | 5.40 | 5.72 | 2.78 | ≤1.00 | ≤1.00 |
| Y. enterocolitica | − | 9.04 | 9.11 | 6.72 | 4.85 | 5.04 |
| B. cereus | + | 6.80 | 7.00 | 2.45 | ≤1.00 | ≤1.00 |
| S. aureus | + | 8.80 | 8.70 | 7.08 | 6.70 | 5.08 |
| S. epidermidis | + | 9.20 | 9.20 | 8.11 | 7.32 | 6.32 |

### Example 9

### Viral Inactivation by Compound VI and Compound X

The porcine parvovirus (PPV) was inactivated by using compound VI and compound X. Treatments were conducted in RS (pH 6.9) at RT with 100 μM using compound VI and compound X and 10% virus spike. Residual compounds were quenched by incubation with 10 mM Na₂S₂O₃ for 2 hours at RT. Virus titers, expressed as Log₁₀TCID₅₀/mL, were determined using the standard endpoint dilution assay with permissive to PPV porcine testis cells. After the incubation of indicator cells for 6 days, infected wells were counted under microscope by visual inspection. To confirm the results, secondary infection using conditioned media from the first plate wells as samples was conducted.

Human respiratory syncytial virus (RSV) was inactivated by using Compound VI or Compound X. For that purpose, sucrose gradient-purified virus was treated with 100 μM of Compound VI and Compound X at RT. At 1, 4, and 6 h of incubation, aliquots were taken and quenched with 10 mM sodium thiophosphate for 30 min at RT. The virus titers were determined using standard 10× serial dilutions in a modified plaque assay. For mock-treated virus no significant changes in RSV infectivity were found even after 6 h incubation at RT (in different experiments, the reduction of titers were in the range of 0.11-0.36 Login PFU/mL).

Bovine viral diarrhea virus (BVDV) was inactivated by using compound VI and compound X. The protocol used for PPV inactivation was adopted from the BVDV inactivation except the indicator cells, which were bovine turbinate cells.

The results of the experiments are shown in Table 2. As much as 5 to 7 log reduction of the virus titers were observed, and all viruses were killed to below the limit of detection after 6 h incubation with compound VI.

TABLE 2
Kinetics of PPV, BVDV, and RSV inactivation at RT with 100M
compounds VI and X

| Incub. | PPV^{a} | | BVDV^{a} | | RSV^{b,c} | |
|---|---|---|---|---|---|---|
| Time, | Cmpd. | Cmpd. | Cmpd. | Cmpd. | Cmpd. | Cmpd. |
| h | VI | X | VI | X | VI | X |
| Ctr., | 6.17 | 6.17 | 5.22 | 5.22 | 7.60 | 7.60 |
| 6 h | | | | | | |
| 1 | 2.95 | 3.09 | 3.27 | 3.88 | 4.52 | 4.95 |
| 3 | 0.85 | 2.72 | 1.30 | 2.94 | 1.89 | 3.48 |
| 6 | BLD | 0.78 | BLD | 0.84 | BLD | 1.95 |

^{a}Titers are expressed as Log₁₀TCID₅₀/mL;
^{b}Titers are expressed as Log₁₀PFU/mL;
^{c}Time points were 2, 4, and 6 h

### Example 10

### Bacterial Inactivation by Compound VI and Compound X in Whole Blood of WB), Leukodepleted Blood (LB), and Red Blood Cells Concentrate (RBCC)

Two G− species, Y. enterocolitica and P. fluorescens, both psychrophiles, and two G+ bacteria, S. epidermids and B. cereus, all known blood contaminants, were used in this study. All blood samples were spiked with approximately 0.1% bacterial stock suspension prepared in RS and left for equilibration at RT for 30 min. Freshly grown overnight bacterial cultures were used for each spiking. Compound VI and compound X were added to spiked blood to final concentrations of 100, 250, and 500 μM. Control samples (Ctr) received solvent only. Incubation was carried out at RT for 6 h followed by the addition of an inactivator, 100× Na thiosulfate, and additional incubation at RT for 2 h. After the incubation and quenching, aliquots were taken for serial dilutions and plate drop-counting and the bacterial growth-promoting solution (containing tryptone, peptone, yeast extract and casamino acids) was added to the remaining volumes. The growth/no growth results were confirmed by streaking agar plates.

Table 3 reflects the results of typical inactivation experiments in WB, LB, and RBCC, respectively.

TABLE 3
Inactivation of selected bacteria by Compound VI and Compound X
in WB, LB, and RBCC for 6 li at RT.

| | | | | Compound | | | Compound | | |
|---|---|---|---|---|---|---|---|---|---|
| | | TO | | VI, 1AM | | | X, iiM | | |
| Species | medium | CFU/mL | Ctr | 100 | 250 | 500 | 100 | 250 | 500 |
| B. cereus | WB | 1.2-102 | + | + | − | − | − | − | − |
| | LB | 6.0.101 | + | − | − | − | − | − | |
| | RBCC | 1.2.102 | ± | + | − | − | ± | − | |
| P. fluorescens | WB | 2.8.102 | + | − | − | − | − | − | |
| | LB | 3.6.102 | + | − | − | − | − | − | |
| | RBCC | 2.6.102 | ± | − | − | − | − | − | |
| S. epidermidis | WB | 2.4.102 | + | − | − | − | − | − | |
| | LB | 1.6-102 | + | − | − | − | − | − | |
| | RBCC | 7.3.102 | + | − | − | − | − | − | |
| Y. enterocolitica | WB | 1.0.102 | + | − | − | − | − | − | |
| | LB | 1.0.102 | + | − | − | − | − | − | |
| | RBCC | 1.6-102 | + | − | − | − | − | − | |

“+” Growth;
“−” No growth

### Example 11

### Viral Inactivation by Compound VI and Compound X in Whole Blood (WB), Leukodepleted Blood (LB), and Red Blood Cells Concentrate (RBCC)

All blood samples were spiked with approximately 20% viral stocks prepared in RS and left for equilibration at RT for 30 min. Viral inactivation study with BVDV or PPV was performed similarly to bacterial inactivation protocol in Example 10. The virus titers (expressed as Log₁₀ TCID₅₀/mL) were determined at TO and after 6 h incubation as described in Example 9. Results for BVDV and PPV inactivation are presented in Table 4.

TABLE 4
Log Reduction (Standard Deviation) of BVDV and PPV by Compound VI and Compound X
in WB, LB, and RBCC

| | Conc. | WB | | LB | | RBCC | |
|---|---|---|---|---|---|---|---|
| Treatment | (μM) | BVDV | PPV | BVDV | PPV | BVDV | PPV |
| Control | 0 | 0.3 (0.2) | 0.4 (0.3) | 0.1 (0.1) | 0.3 (0.1) | 0.3 (0.1) | 0.2 (0.1) |
| Compound VI | 100 | 5.0 (0.9) | 5.4 (0.6) | 5.2 (0.6) | 5.3 (0.5) | 4.7 (1.0) | 5.6 (0.5) |
| | 250 | 6.0 (0.7) | 6.4 (0.4) | 6.0 (0.7) | 6.4 (0.2) | 5.9 (0.8) | 6.4 (0.5) |
| | 500 | 6.2 (0.4) | 7.2 (0.2) | 6.2 (0.4) | 7.2 (0.2) | 6.2 (0.4) | 7.1 (0.2) |
| Compound X | 100 | 4.3 (0.6) | 4.2 (0.6) | 3.7 (0.4) | 4.7 (0.9) | 3.0 (1.1) | 4.3 (0.6) |
| | 250 | 5.8 (1.0) | 5.8 (0.5) | 5.2 (0.4) | 5.8 (0.6) | 4.3 (1.3) | 5.7 (0.7) |
| | 500 | 6.2 (0.4) | 6.9 (0.1) | 6.2 (0.4) | 7.0 (0.3) | 5.3 (1.3) | 7.0 (0.3) |

### Example 12

### Inactivation of RSV by Compound XVI

Inactivation of RSV with different concentrations of Compound XVI at RT and 40° C. was performed as described in Example 9. The results are shown in Table 5.

TABLE 5
Concentration-dependent inactivation of RSV in Ringer's/Lactate
solution by Compound XVI (6 h)

| Parameters/ | | | Compound XVI (μM) | | | |
|---|---|---|---|---|---|---|
| Temperature | TO* | Ctr* | 100 | 250 | 500 | |
| Titer* | RT | 7.00 | 6.83 | 1.98 | 1.50 | <0.52 |
| Inactivation† | | NA | 0.17 | 5.02 | 5.50 | >6.48 |
| Titer* | 40° C. | 7.00 | 5.18 | <0.52 | <0.52 | <0.52 |
| Inactivation† | | NA | 1.82 | >6.48 | >6.48 | >6.48 |

*All titers are expressed as Log₁₀ PFU/mL
†Inactivation was calculated as a difference between TO titer and corresponding titer at specified treatment conditions.

### Example 13

### Inactivation of BVDV and PPV by Compound VI in Heat-Inactivated Fetal Bovine Serum (FBS)

Aliquots of FBS were spiked with 5% (vol/vol) of BVDV and PPV stocks and allowed to equilibrate for 60 min at RT. Compound VI, 10 mM in phosphate buffer (pH 6.9) was added to the spiked FBS to a final concentration of 10011M and all aliquots were treated as described in Table 6.

TABLE 6
Controls and treatment conditions for virus inactivation

| | |
|---|---|
| Control 1 | Virus stock spiked PBS |
| Control 2 | Spiked FBS without further incubation |
| Control 3 | Spiked FBS incubated at 40° C. for 60 min |
| Control 4 | Spiked FBS, incubated at 40° C. for 60 min and passed |
| | through solid phase agent cartridge |
| Treatment | Spiked FBS, treated with 100 μM Compound VI at 40° C. for |
| | 60 min, and passed through solid phase agent cartridge |

Virus-spiked serum samples were treated with 100 μM Compound VI at 40±1° C. for 60 min. Aliquots from all samples (Controls 1-4 and Treatment sample) were serially diluted (1:5 or 1:10) in DMEM without serum and 25 μL from each dilution were plated in triplicates onto their respective indicator cells in 96-well plates. Plates were incubated at 37° C. in a 5% CO2-incubator for 60 min to allow virus adsorption. To increase the limit of detection, non-diluted samples were additionally used to infect host cells in 24-well plates or in 10 cm Petri dishes. After the adsorption, all wells were filled with DMEM/5% FBS without aspiration of 25 μL dilutions and plates were further incubated at 37° C. in a CO₂-incubator for 6-7 days. The development of viral cytopathic effect in each well was detected by visual inspection and used to calculate the respective virus titers expressed as Log₁₀TCID₅₀/mL. The limit of detection was 0.2 infective particles per mL. In some cases, in order to confirm the results of the assay, supernatant from inoculated wells was collected after 6-7 days and used to infect fresh cells in 24-well plates.

The results of the experiments presented in Table 7 demonstrate that the treatment with compound VI effectively inactivated both BVDV and PPV to below the limit of detection of the assay.

TABLE 7
Inactivation of BVDV and PPV in FBS with 100 μM Compound
VI during 60 min incubation at 40° C.

| | PPV | BVDV |
|---|---|---|
| | (Log₁₀) TCID₅₀/mL ± SD) | (Log₁₀ TCID₅₀/mL ± SD) |
| Control 1 | 5.10 ± 0.20 | 4.87 ± 0.31 |
| Control 2 | 5.03 ± 0.31 | 4.73 ± 0.25 |
| Control 3 | 5.00 ± 0.20 | 4.37 ± 0.15 |
| Control 4 | 4.97 ± 0.25 | 3.70 ± 0.20 |
| Treatment | BLD* | BLD |

*BLD, Below the Limit of Detection, <−0.7 Logic) TCID₅₀/mL

### Example 14

### Compounds of Structure I as Protozoan and Fungal Inactivators

Inactivation of blood-borne parasites, Plasmodium falciparum 3D7 and Babesia divergens Rouen, was conducted in fresh human red blood cells for 24 hours at physiological temperature. Compound XIV at concentrations 250 μM displayed strong anti-parasitic activity reducing the number of viable plasmodium organisms by order of 7 plus logs and Babesia by 8 logs. Above six logs inactivation of Candida albicans, a representative of pathogenic fungi, and three logs inactivation of Tetrahymena thermophila, a model organism for ciliated protozoa, were achieved by compound XIV at 250 μM in their respective growth media.

### Example 15

### Neutralization of Compound X by Ethyl-2-Mercaptoacetate

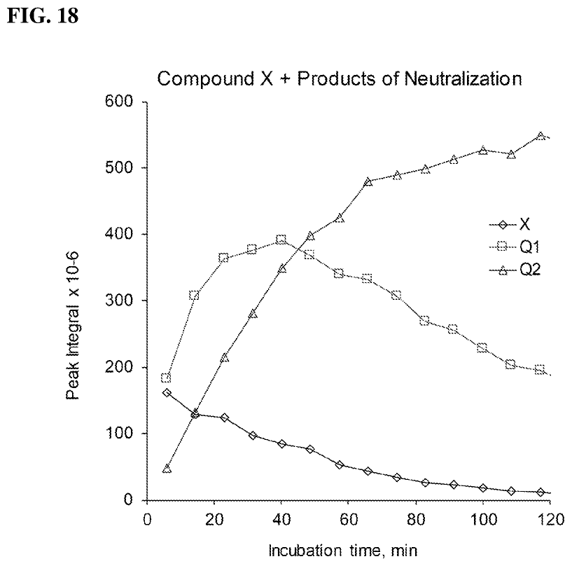

100 μM solution of compound X in phosphate buffered saline was incubated at room temperature with 10 mM of ethyl-2-mercaptoacetate, and the change of the concentration of compound X, as well as the formation of the intermediate compound of neutralization (XXI) and the final compound of neutralization XXII was determined by LCMS analysis of the mixture. The reaction scheme of neutralization is presented below. The peak areas of compound X, intermediate neutralization product, Q1 (compound XXI) and final neutralization product, Q2 (compound XXII) are presented in the Table 8 below, and in FIG. 18.

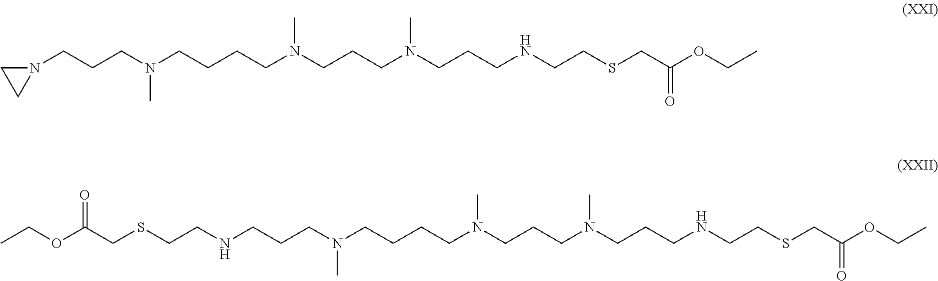

TABLE 8
LCMS analysis of the reaction of neutralization of compound
X by ethyl 2-mercaptoacetate in PBS and room temperature.

| | Peak Areas | | |
|---|---|---|---|
| Time, min | X | Q1, XXI | Q2, XXII |
| 6 | 161.9 | 181.6 | 48.7 |
| 14.5 | 128.8 | 307 | 131.9 |
| 23.1 | 123.1 | 363.5 | 214.4 |
| 31.6 | 96.3 | 375.8 | 280.7 |
| 40.2 | 85 | 390.1 | 349.3 |
| 48.7 | 76.9 | 368.5 | 397.1 |
| 57.3 | 52.9 | 338.6 | 424.3 |
| 65.8 | 42.7 | 331.1 | 479.4 |
| 74.3 | 33.1 | 306.5 | 489.3 |
| 82.9 | 26 | 268.6 | 498.6 |
| 91.4 | 23.2 | 256 | 513.2 |
| 100.0 | 17.4 | 227.8 | 527 |
| 108.5 | 14 | 202.2 | 520.5 |
| 117.1 | 11.5 | 195 | 548.8 |
| 125.6 | 7.6 | 174 | 536.5 |

### Example 16

### Neutralization of Residual Compound VI by Sodium Thiosulfate

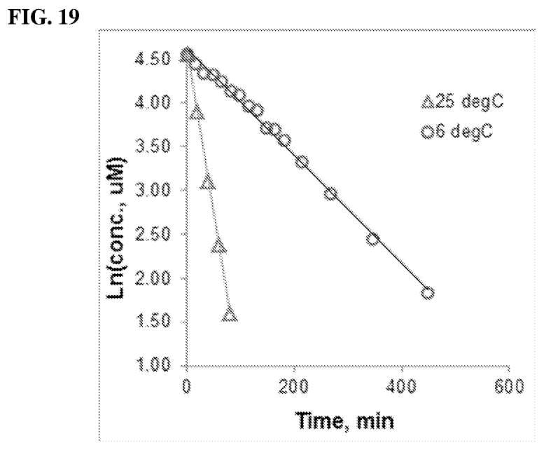

Studies of the reaction of sodium thiosulfate with the compounds of Structure I showed that Na₂S₂O₃ reacts quickly with the aziridine groups of the compounds, opening the ring and converting them to biologically well-tolerated thiosulfate esters, which are expected to be subject to fast renal excretion. The rate of reaction of Compound VI, 100 μM with 1 mM Na₂S₂O₃ in PBS was determined by LCMS analysis of the reaction mixture (FIG. 19). The reaction follows first-order kinetics with rate constants of 0.00614 min⁻¹ at 6° C., and 0.0379 mind at 25° C. At this reaction rate, the half-live of compound VI in 10 mM Na₂S₂O₃ and 25° C. will be 1.83 min, which after 2 h will result in 5.5×10⁻¹⁹ M residual compound VI concentration. LCMS analysis of the reaction product confirmed that it was the bisthiosulfate ester (compound XXIII formed by reacting of compound VI with two molecules of Na₂S₂O₃.

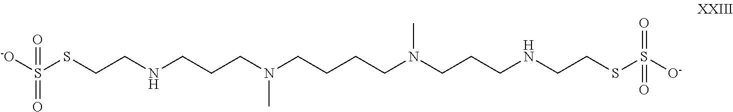

### Example 17

Neutralization of Compound X with Methyl Thiosalicylate

To 178 μL of phosphate buffered saline were added 2 μL of 10 mM solution of compound X in methanol and 20 μL of 100 mM solution of methyl thiosalicylate acid in methanol, which resulted in 100 μM inactivator and 10 mM methyl salicylate final concentrations. This solution was analyzed by liquid chromatography mass spectrometry for change of the concentration of the compound X and formation of the covalent adducts (Compounds XXIV and XXV) between compound X and the methyl thiosalicylate, that is schematically illustrated herein.

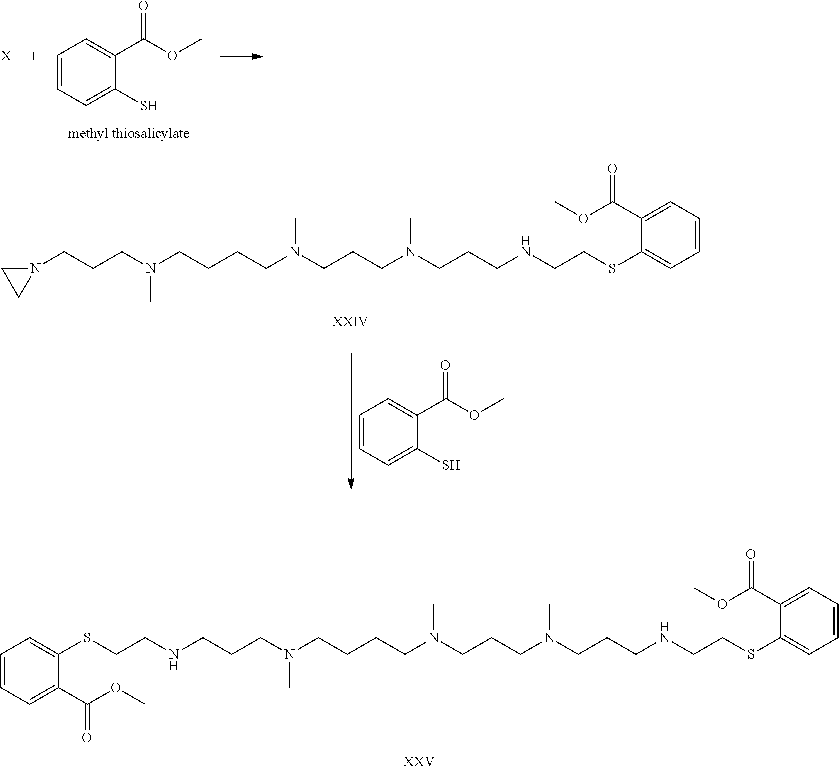

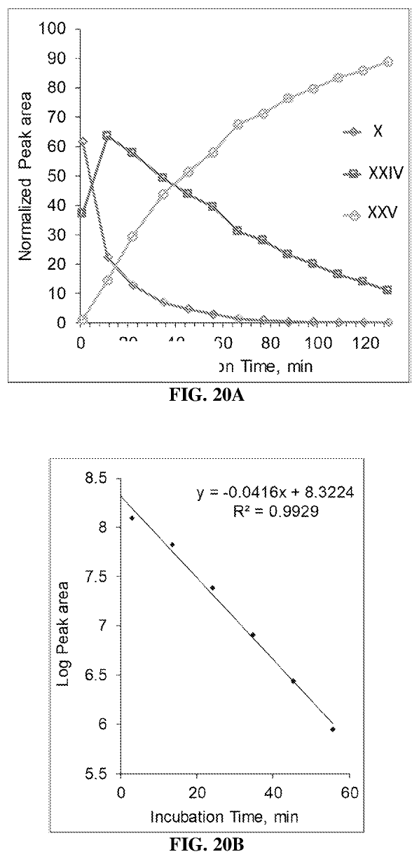

The results, plotted in FIG. 20A, show that the concentration of the compound X decreases due to formation of the intermediate compound XXIV, which further converts to compound XXV. The rate of neutralization of compound VI can be determined by plotting of logarithm of compound X concentration, determined by its peak area against the time of incubation. This plot, shown in FIG. 20B, reveals a liner dependence, indicating a first order reaction kinetics with a first order rate constant K=−0.0416 min⁻¹, corresponding to compound X half-life of T_{1/2}=16.6 min.

### Example 18

Neutralization of Compounds of Structure I with Thiophenol

To 178 μL of phosphate buffered saline were added 2 μL of 10 mM solution of compound X in methanol and 20 μL of 100 mM solution of thiophenol in methanol, resulting in 100 μM inactivator and 10 mM thiophenol final concentrations. This solution was analyzed by liquid chromatography mass spectrometry for change of the concentration of compound X and formation of the covalent adducts, compound XXVI and compound XXVII between the compound X and the thiophenol, that is schematically illustrated herein.

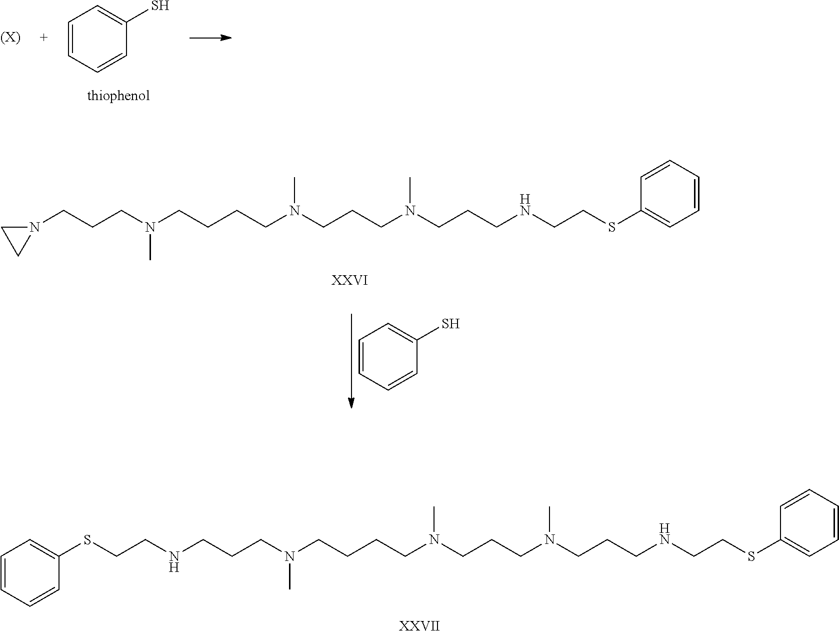

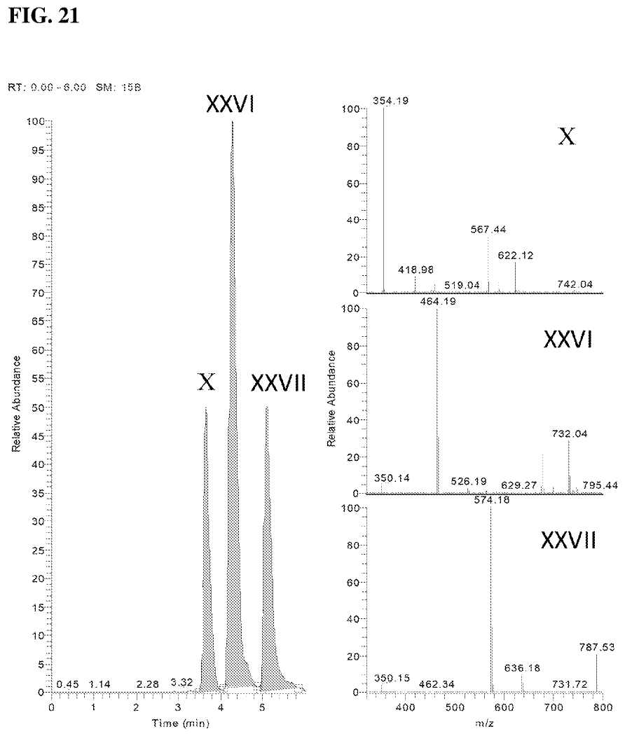

In FIG. 21 is shown the result of the LCMS analysis of compound X with thiophenol at different time points. In the left panel of FIG. 21 is shown the total ion current mass chromatogram of the LCMS analysis where the peaks correspond to compounds X, XXVI and XXVII. In the right panel are shown the mass spectra of the corresponding peaks. The analysis reveals that after 1 min and 40 sec (100 sec) compound X is neutralized by a significant degree: the ratio of the peak areas of compounds X, XXVI and XXVII is 21:52:27, respectively. The ratio of those peaks after 10 min is 3:29:68, and after 20 min is 0.5:16:83.5 indicating quick conversion of compound X to mono- and di-covalent adducts XXVI and XXVII.

### Example 19

### Preparation of Solid Phase Agent Having Thiosulfonate Functional Groups XXVIII and its Use for Neutralization of Compound VI

Sulfonylchloride functionalized divinylbenzene crosslinked polystyrene resin (Sigma-Aldrich Cat. no. 498211-5g) was mixed with 5 ml 2M sodium hydrogen sulfide solution (prepared by saturation of sodium sulfide nonahydrate solution in water with hydrogen sulfide) under argon. The mixture was sonicated for ca. 3 min and then stirred at 55° C. for 4 h. After that the resin was filtered and washed three times with deaerated water, there times with deaerated methanol, and two times with deaerated ether. The resin was dried under stream of argon and then under vacuum. Obtained, 1.039 g dry resin (compound XXVIII), thiosulfonate functionalized polystyrene/divinylbenzene resin. An aliquot of compound XXVIII was added to a solution of compound VI, 100 μM in PBS. LCMS analysis demonstrated time dependent decreasing of the concentration of compound VI in the mixture.

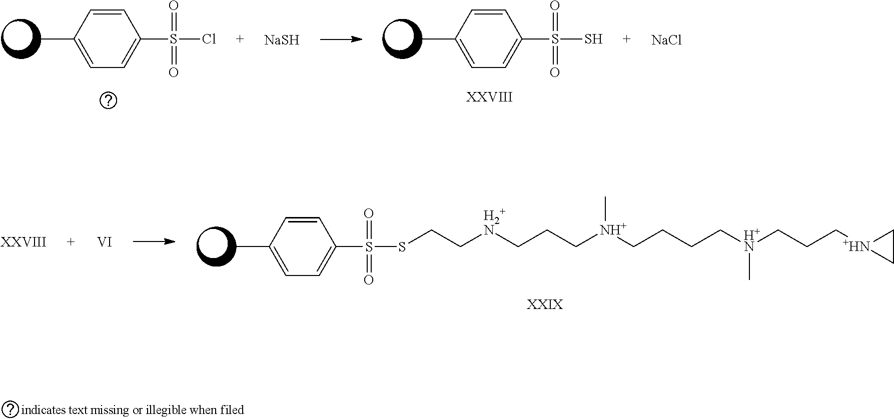

A reaction scheme of the preparation of the solid phase agent XXVIII and the reaction of neutralization and covalent sequestration of compound VI by the solid phase agent XXVIII with formation of compound XXIX (e.g. compound VI covalent adduct with the solid phase agent XXIX) is shown herein.

### Example 20

### Preparation of Mercaptophenyl Groups Functionalized Methacrylate Resin Based Solid Phase Agent XXX and its Use for Neutralization and Covalent Sequestration of Compound VI

4-Mercaptophenylacetic acid (Sigma-Aldrich catalog No. 653152-5G), 400 mg was dissolved with 2 ml dimethyl sulfoxide and the solution was left overnight at room temperature. The formed dimethyl sulfide was removed under 10 torr vacuum, and the excess of dimethyl sulfoxide was removed under 0.05 torr vacuum at 45° C. overnight. This resulted in quantitative yield of the disulfide of 4-mercaptophenylacetic acid as a waxy yellowish solid.

Aminoethyl groups functionalized methacrylate resin (Purolite Ltd, Llantrisant, Wales, UK, Product No. D6195, trade name Chromalite MAM2, 0.5 mmol amino groups per ml wet resin, 68% moisture), 300 mg, was dried by three evaporation from 2 ml dry N,N-dimethylformamide under vacuum at 35° C. The dry resin was suspended in 1 ml dry N,N-dimethylformamide and to this suspension was added a solution of the disulfide of 4-mercaptophenylacetic acid, 370 mg in 1 ml dry tetrahydrofuran. To this suspension was added, under stirring, benzotriazol-1-yl-oxytripyrrolidinophosphonium hexafluorophosphate, 172 mg, followed by dropwise addition of 172 μL of N,N-diisopropylethylamine and the reaction mixture was sealed under argon. After 24 h a solution of dithiothreitol, 330 mg in 1 ml deionized and deaerated water was added on stirring and after 10 min the resin was recovered by vacuum filtration and washed repeatedly with deaerated acetonitrile, tetrahydrofuran, methanol, 0.2 mM diethylenetriaminepentaacetic acid (DPTA) and purged with argon to obtain 314 mg of wet mercaptopheny groups functionalized methacrylate resin. The load of mercapto groups on the product, compound XXX, was determined using the Elman's procedure (Riener, C. K.; Kada, G.; Gruber, H. J., Anal. Bioanal. Chem., 2002, 373, 266-76) and was 0.21 mmol per gram of wet resin. The moisture content was 71%. An aliquot of compound XXX was added to a solution of compound VI, 100 μM in PBS. LCMS analysis of this mixture demonstrated time dependent decreasing of compound VI in the mixture.

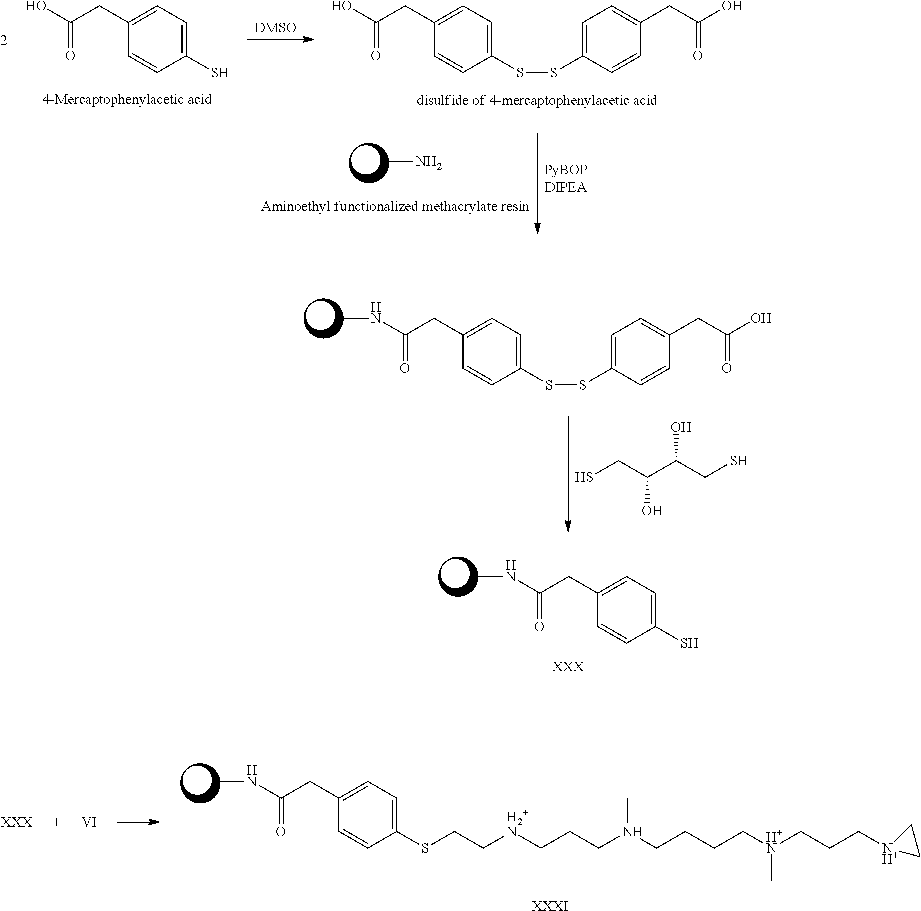

The above reaction scheme illustrates the synthesis of the solid phase agent XXX and its reaction with compound VI, with formation of compound XXXI (e.g. compound VI covalent adduct XXXI with the solid phase agent XXX).

### Example 21

### Preparation of Thiophenol Groups Functionalized Polyethylene Glycol Grafted Polystyrene-Divinylbenzene Resin XXXII and its Use for Neutralization of Compound VI

4-Mercaptophenylacetic acid, 900 mg was added to a solution of 1.60 g of triphenylmethyl chloride in 50 ml anhydrous dichloromethane. The mixture was stirred under argon for 3 h at RT. Water, 30 ml, was added, and the mixture was stirred for 5 min. The dichloromethane layer was separated, dried over sodium sulfate and evaporated under vacuum to give 2.3 g of crude product as a white solid. This material was purified by silica gel chromatography with a gradient from chloroform to chloroform/methanol 10:1 to give 1.62 g, 74% of 2-(4-(triphenylmethylthio)phenyl)acetic acid.

Tentagel S NH2 resin, 200 mg (Rapp Polymere GmbH, Tuebingen, Germany, product No. 530132, divinylbenzene cross-linked polystyrene resin grafted with amino group terminated polyethylene glycol) was swollen for couple of hours in 5 ml dry N,N-dimethylformamide and then the excess of solvent was pipetted off Benzotriazol-1-yl-oxytripyrrolidinophosphonium hexafluorophosphate, 151 mg, 2-(4-(triphenylmethylthio)phenyl)acetic acid, 119 mg, and anhydrous 1-hydroxybenzotriazole, 44 mg, were dissolved in 1.2 mL dry N,N-dimethylformamide. Diisopropylethylamine, 75 mg, 101 μL were added on stirring and after 1 min the resulting solution was added to the swollen resin. After 2 h shaking at RT, the resin was filtered and washed with N,N-dimethylformamide, 3×2 mL and dichloromethane, 3×2 mL, and then dried under stream of argon. The resin was suspended in 2 mL solution of triisopropylsilane, 2.5% and water, 2.5% in tetrahydrofuran. After 2 min the resin was filtered under argon and the deblocking was repeated. The resin was then filtered under argon, washed three times with 3 ml deaerated acetonitrile and dried under stream of argon to obtain 203 mg of mercaptophenyl groups bearing TentaGel S resin (e.g. compound XXXII). The mercapto groups load was determined using the Elman's procedure and was 0.12 mmol per gram of dry resin. An aliquot of compound XXXII was added to a solution of compound VI, 100 μM in PBS. LCMS analysis demonstrated time dependent decreasing of compound VI in mixture.

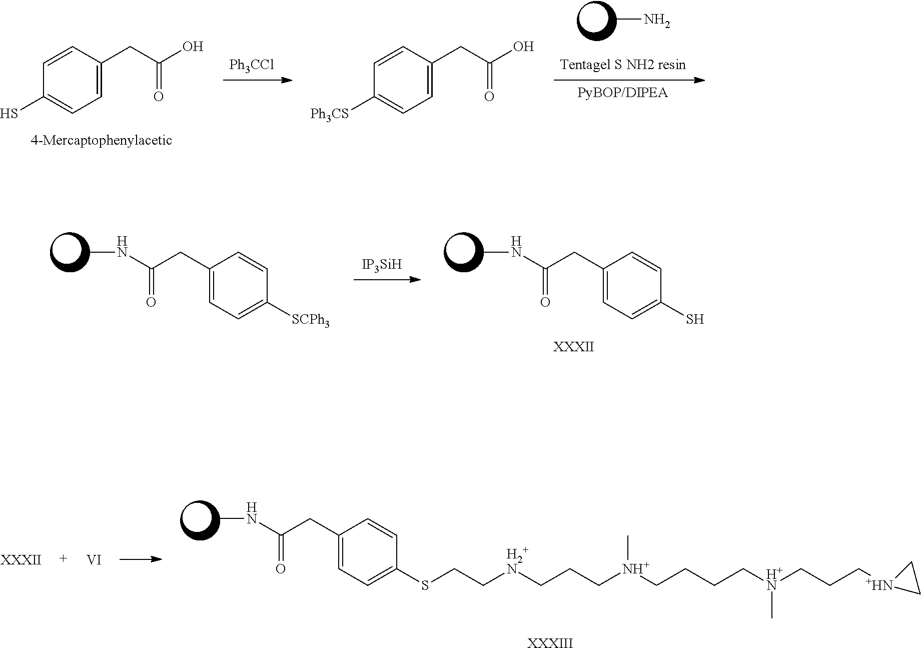

The above reaction scheme illustrates the synthesis of the solid phase agent XXXII and its reaction with compound VI, with formation of adduct compound XXXIII (e.g. compound VI covalent adduct XXXIII with the solid phase agent XXXII).

### Example 22

Preparation and Use of a Solid Phase Agent that Binds Compound(s) of Structure I and their Neutralization or Decomposition Products Through Ion Pairs Formation.

Purolite NRW160 polystyrene divinylbenzene cross-linked resin functionalized with sulfonic groups in the H⁺ form, 500 g was transferred into the Na⁺ form by the following steps: The beads were washed on a vacuum filter and under a sterile hood with 3 volumes of saturated NaCl solution, followed by two volume of 1 M NaOH. After NaOH sterilization beads were washed by sterile deionized water until the pH of the rinsings became neutral. The beads were incubated with two volumes of methanol for 15 min and after removing of methanol were rinsed again with three volumes of sterile deionized water. After final incubation in methanol (2 volumes) the alcohol was removed by filtration and beads were dried under the vacuum.

Dry beads, 50 mg were added to one mL of 100 μM solution of compound X in phosphate buffered saline. LCMS analysis of this mixture showed that the concentration of compound X in the supernatant was reduced to below 30 nM.

### Example 23

### Preparation of Solid Phase Agent Cartridges

Empty polypropylene cartridges 5×50 mm, 20×120 mm 20×200 mm (diameter×length, mm, Cat. Nos. PF-DLE-F0004; PF-DLE-F0025 and PF-DLE-F0040, Interchim, Montlucon Cedex, France) fitted with bottom polypropylene filter were loaded with solid phase agent. In the case of dry solid phase agent, the cartridges were filled ⅔ of their capacity in order to provide for the swelling of the beads upon wetting. The top of the cartridges was fitted with another polypropylene filter disk and the cartridges were sealed and stored at room temperature (dry solid phase agent) or refrigerated (wet solid phase agent). The cartridges can be integrated into the treatment closed systems, as illustrated in FIGS. 2, 3, 5-8.

### Example 24

Preservation of Cell Culture-Supporting Properties of Animal Sera Treated with Compound VI

Heat-Inactivated fetal bovine serum (FBS, Cat. No. 89510-188, VWR) and heat inactivated horse serum (HS, Cat. No. H1138, Sigma) were incubated with 100 μM of compound VI for 60 min at 40±1° C. in 50 mL sterile conical tubes. Treatment-control sera were incubated for 60 min at 40±1° C. with compound VI diluent only. After the incubation, compound VI was removed from treated sera using cartridges filled with solid phase agent, which were prepared as described in Examples 22 and 23. After the cartridge filtration the sera were filter-sterilized using 0.2μ syringe filters. Control sera were not incubated at 40° C. or exposed to solid phase agent but were filter-sterilized.

These sera were used to supplement cell growth media at three different concentrations, 5%, 10%, and 20%. The ability of these media to support growth of bovine turbinate cells (BTT, fibroblast morphology), porcine testis cells (PT, epithelial), and two human cell lines: A172 (glioblastoma, astrocyte-like cells) and MCF7 (epithelial breast cancer cells) was evaluated.

Cell Growth curves: BTT, PT, A172, and MCF7 cells at early stage of confluency were trypsinized and plated into 48-well plates in DMEM supplemented with treated or control sera as described above. Media were changed every day. Viable cells were counted every 24 h with standard hemocytometer using trypan blue exclusion. Results are presented as average number of cells per well. At least three wells were used for each dilution.

Clonal growth: BTT, PT, A172, and MCF7 cells at early stage of confluency were trypsinized, serially diluted (1:2) and plated in six replicates into 96-well plates in DMEM supplemented with treated or control sera as described above. Media were changed every two days for 16 days. The presence of clonal growth was determined by visual inspection of each well. Results are presented for the last four dilutions where cell growth was observed as number of wells with growth from the total of the six replicates for each dilution.

Long term culturing: BTT, PT, A172, and MCF7 cell lines were propagated in media supplemented with control, or treated FBS or HS (BTT cells only) in the manner described above for 10 passages at 3-4 days intervals. Cell and monolayer morphology were monitored daily using phase contrast microscopy.

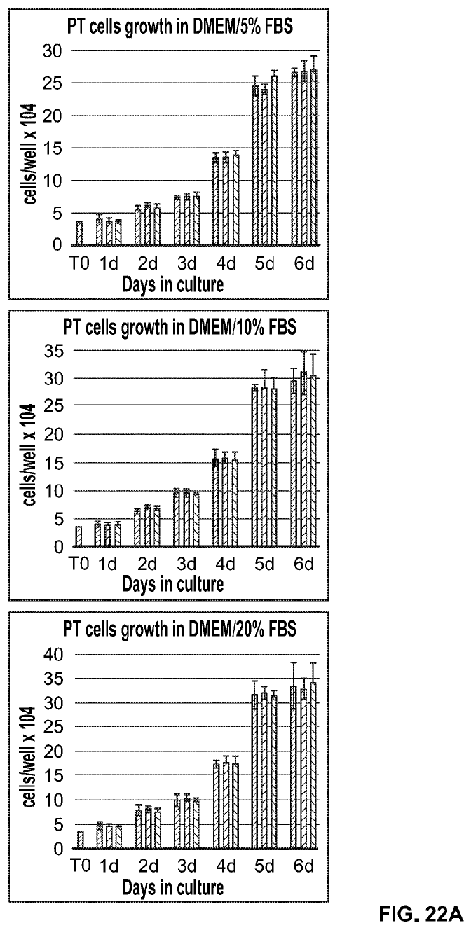

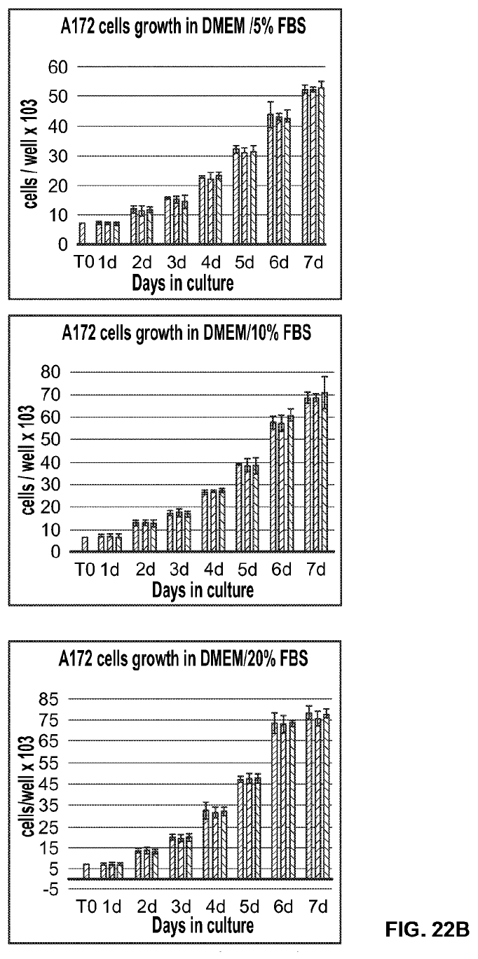

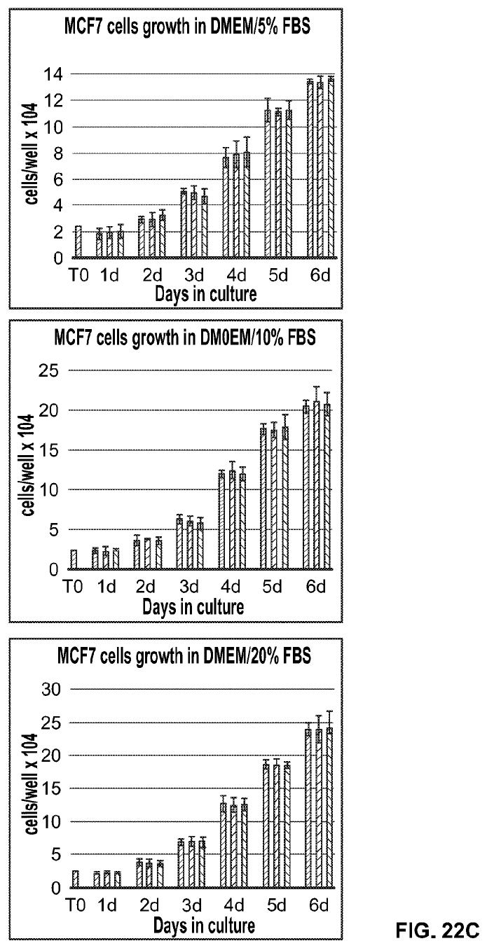

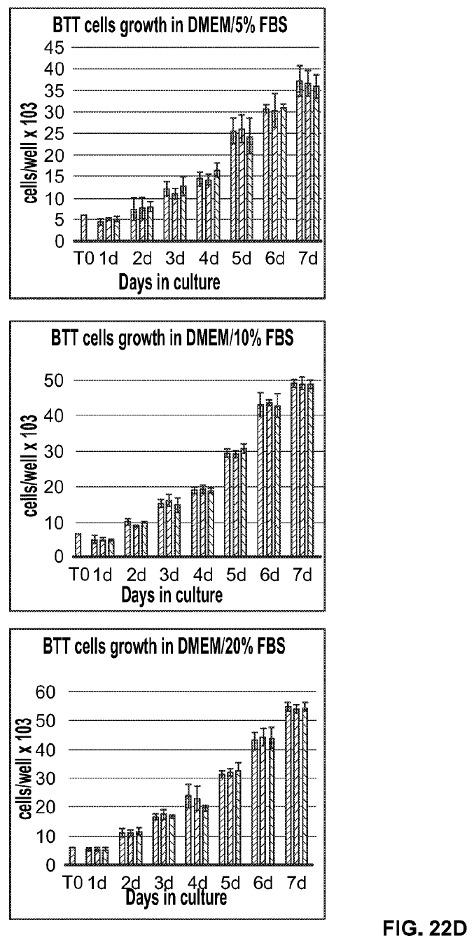

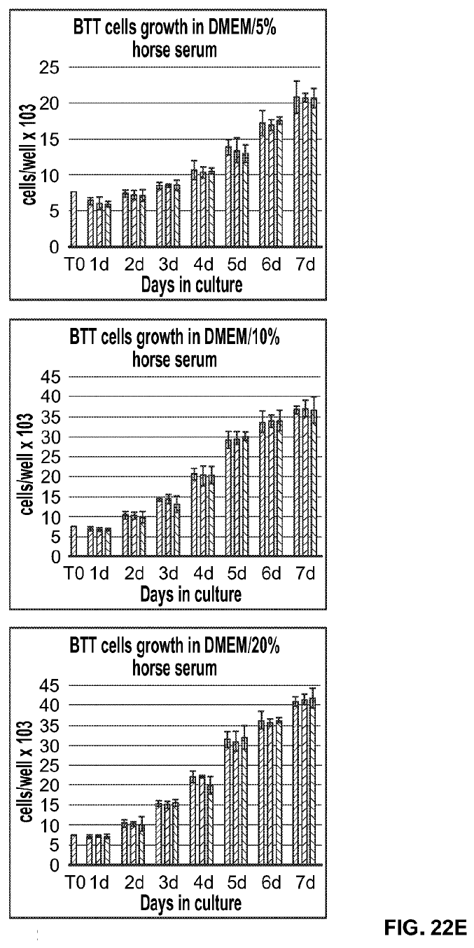

Cell Growth Results: Typical growth curves are presented in FIGS. 22A-22E. All growth curves displayed a similar pattern: a classic lag-phase was initially observed with all cell lines and in all media, gradually followed by a log-stage of growth. As expected, the highest growth rates were found for all cell lines cultured in medium with 20% serum. Growth in 10% serum-supplemented medium had intermediate values while cell proliferation in medium with 5% serum was greatly reduced. No statistically significant differences in growth rates between cells grown in the presence of control, mock-treated or compound VI-treated serum were found for all cell lines and for all serum concentrations.

FIGS. 22A-22E shows the effect of mock-treated and Compound VI-treated serum on the growth of four different cell lines in 48-well plates measured over 6-7-day periods. 22A, porcine PT cells; 22B, human A172 cells; 22C, human MCF-7 cells; 22D, bovine BTT cells grown in medium with FBS; 22E, bovine BTT cells grown in medium with HS. TO columns indicate cell numbers in time of plating; First columns in array of three (day 1 to 7)—number of cells in wells containing medium supplemented with control, non-treated serum; second columns in array of three (day 1 to 7)—number of cells in wells containing medium supplemented with mock-treated serum; Third columns in array of three (day 1 to 7) columns—number of cells in wells containing medium supplemented with Compound VI-treated serum. Each time point represents the mean of three wells. Error bars indicate the SD.

Clonal Growth Results: The ability to support cell growth at very low seeding density (clonal growth) is another important characteristic of the sera. Table 9 shows the presence of growth of the serially diluted cells in the four final dilutions. These results indicate that the clonal growth of all four cell lines was not affected by the serum treatment.

TABLE 9
Clonal growth of cells in medium supplemented with control
and Compound VI treated FBS. The presence of growth in the
last four dilutions is shown.

| | | Number of wells with | | | | |
|---|---|---|---|---|---|---|
| | | cell growth in the last | | | | Total for |
| | | four dilutions from | | | | the last |
| Cell line | Serum | total of six replicates | | | | 4 dilutions |
| PT | Control | 5 | 5 | 3 | 0 | 13 |
| | Treated | 6 | 6 | 2 | 0 | 14 |
| BTT | Control | 5 | 5 | 2 | 0 | 12 |
| | Treated | 5 | 4 | 1 | 1 | 11 |
| A172 | Control | 6 | 2 | 2 | 0 | 10 |
| | Treated | 6 | 4 | 1 | 0 | 11 |
| MCF7 | Control | 6 | 3 | 2 | 0 | 11 |
| | Treated | 5 | 2 | 2 | 1 | 10 |

Long Term Culturing Results: No visual differences were observed in cell growth/appearance or the morphology of intermediate or confluent monolayers between cells maintained in the media with Compound VI-treated serum and cells in control medium for 10 consecutive passages.

### Example 25

### Testing of the Ability of Compound VI Treated Fetal Bovine Sera to Preserve its Ability to Support Viral Development and Infectivity

Serially diluted Porcine Parvovirus (PPV, ATCC #VR-742) and bovine viral diarrhea virus (BVDV, ATCC #VR-534) stocks were added to porcine testis cells (PT, PT; ATCC #CRL-1746) and bovine turbinate cells (BTT, ATCC; #CRL-1390), respectively and, after adsorption, medium supplemented with control or compound VI-treated FBS prepared as described in Example 24 was added. Aliquots from all samples spiked with viruses (treated, mock-treated or non-treated serum) were serially diluted (1:5 or 1:10) in DMEM without serum and 25 μL from each dilution were plated in triplicates onto their respective indicator cells in 96-well plates. Plates were incubated at 37° C. in a 5% CO2-incubator for 60 min to allow virus adsorption. To increase the limit of detection, non-diluted samples were additionally used to infect host cells in 24-well plates or in 10 cm Petri dishes. After the adsorption, all wells were filled with DMEM15% FBS without aspiration of 25 μL dilutions and plates were further incubated at 37° C. in a CO2-incubator for 6-7 days. The development of viral cytopathic effect in each well was detected by visual inspection and used to calculate the respective virus titers expressed as Log₁₀TCID₅₀/mL. The limit of detection was 0.2 infective particles per mL. In some cases, in order to confirm the results of the assay, supernatant from inoculated wells was collected after 6-7 days and used to infect fresh cells in 24-well plates.

The results of virus titration shown in Table 10 indicate that control medium supplemented with untreated FBS and medium supplemented with compound VI-treated serum have essentially the same viral infection support properties in the tested cells.

TABLE 10
Comparison of viral titers determined in DMEM
supplemented with 5% control (untreated) FBS
versus DMEM/5% compound VI-treated FBS.

| | PPV | BVDV |
|---|---|---|
| Serum used | (Log₁₀) TCID₅₀/mL ± SD) | (Log₁₀) TCID₅₀/mL ± SD) |
| Control | 5.03 ± 0.21 | 4.53 ± 0.23 |
| Treated | 4.97 ± 0.25 | 4.60 ± 0.26 |

### Example 26

### Quality of Compounds of Structure I Treated Whole Blood and Red Blood Cells (RBCs)

Ten mL samples of whole blood or red blood cells concentrate (RBCC, 25 mL) were treated with 500 μM compound VI for 6 hours at RT. The residual compound VI was neutralized by the same volume of 10 mM sodium thiosulfate for 2 hours at RT. For controls, identical samples of whole blood or RBCC were treated with saline and sodium thiosulfate without compound VI or with saline only without compound VI and/or thiosulfate. Aliquots of whole blood and RBCC from each sample were subjected to complete blood count and biochemistry analysis using IDEXX Procyte Dx Hematology Analyzer and IDEXX Catalyst Dx Chemistry Analyzer according to manufacturer recommendations. The samples were analyzed immediately after the treatment and re-analyzed after one week for whole blood and every week for 5 weeks of storage at 4-6° C. for RBCC. The following parameters were measured: RBC number, hemoglobin, hematocrit, mean corpuscular volume, mean corpuscular hemoglobin, red cell distribution, reticulocyte count, platelets, mean platelet volume, white blood cells, neutrophils, lymphocytes, monocytes, eosinophils, basophils, chloride, potassium, sodium, glucose, and lactate concentrations. No differences in cellular or biochemical characteristics, within the accuracy and precision of the analyzer, between the treated samples and controls were found in all measured parameters (RBC number, hemoglobin, hematocrit, mean corpuscular volume, mean corpuscular hemoglobin, red cell distribution, reticulocyte count, platelets, mean platelet volume, white blood cells, neutrophils, lymphocytes, monocytes, eosinophils, basophils, chloride, potassium, sodium, glucose, and lactate concentrations) after weekly testing.

### ASPECTS OF THE INVENTION

The invention provides the below, non-limiting aspects:

Aspect I. A method for inactivation or reduction of pathogens or undesired organisms from a sample, comprising:
(i) treatment of the sample with a compound having Structure I:

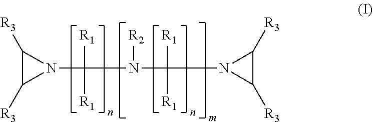

wherein:
each R₁ is independently selected for each occurrence from H, CH₃, CH₂CH₃, CH(CH₃)₂, Cl, F, an alkyl group, an alkenyl group, a phenyl group, an alkyloxy group, an acyloxy group, or substituted alkyl group,
each R₂ is independently selected for each occurrence from H, CH₃, CH₂CH₃, CH(CH₃)₂, an alkyl group, an alkenyl group, a phenyl group, a cycloalkyl group, an alkyloxy group, or substituted alkyl, substituted alkenyl, substituted cycloalkyl or substituted phenyl group, or a moiety of Structure II:

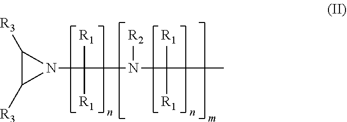

each R₃ is independently selected for each occurrence from H, CH₃, CH₂CH₃, CH(CH₃)₂, Cl, F, an alkyl group, an alkenyl group, a phenyl group, an alkyloxy group, an acyloxy group, or other substituted alkyl group;
each n is independently for each occurrence 3, 4, or 5;
each m is independently for each occurrence 1, 2, 3, 4, 5, 6, 7, 8, 9 or 10;
or a chemically acceptable salt, hydrate, or solvate thereof;
(ii) incubation for sufficient time for inactivation or reduction of pathogens or undesired organisms from the sample;
(iii) treatment of the sample with a one or more neutralizing agents which eliminate or reduce the toxicity or other undesirable properties of the compound with Structure I.

Aspect 2. The method according to Aspect 1, wherein the compound of Structure I has the Structure IA:

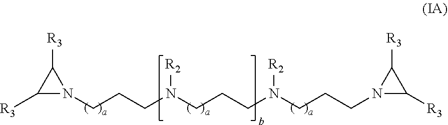

wherein:
each R₂ is independently selected for each occurrence from H, an alkyl group, CH₃, CH₂CH₃, CH(CH₃)₂, an alkenyl group, a phenyl group, a cycloalkyl group, an alkyloxy group, or substituted alkyl, alkenyl, cycloalkyl, phenyl group, or a moiety of Structure IIA:

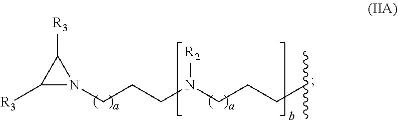

each R₃ is independently selected for each occurrence from H, Cl, F, an alkyl group, CH₃, CH₂CH₃, CH(CH₃)₂, an alkenyl group, a phenyl group, an alkyloxy group, an acyloxy group, or a substituted alkyl group;
each a is independently selected for each occurrence from 1, 2 or 3; and
each b is independently selected for each occurrence from 0, 1, 2, 3, 4, 5 or 6.

Aspect 3. The method according to Aspect 1, wherein the compound of Structure I has the Structure IB:

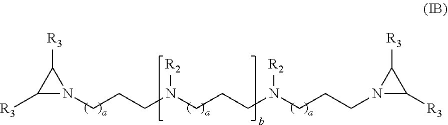

wherein:
each R₂ is independently selected for each occurrence from H, CH₃, CH₂CH₃, or CH(CH₃)₂;
each R₃ is independently selected for each occurrence from H, CH₃, CH₂CH₃, or CH(CH₃)₂;
each a is independently selected for each occurrence from 1, 2 or 3; and
b is selected from 0, 1, 2, 3, 4, 5 or 6.

Aspect 4. The method according to any one of Aspects 1 to 3, wherein the one or more neutralizing agents are nucleophilic compounds which eliminate the alkylating properties of the compound of Structure I, IA or IB by reacting with and opening of the aziridine rings of the compound of Structure I, IA or D3.

Aspect 5. The method of Aspect 4, wherein the one or more neutralizing agents are thiosulfates, preferably sodium thiosulfate, thiophosphates, preferably sodium thiophosphate, thiourea or substituted thioureas, thiocarboxylic acids and salts thereof, dithiocarboxylic acid and salts thereof, thiocarbonate salt, dithiocarbonate salt, salt of thiocarbonate O-esters, salt of dithiocarbonate O-esters, mercaptans or thiols, or their salts, or substituted mercaptans, or substituted thiols, or polymercaptan or polythiols and their salts, or any combination thereof, or organic polymer soluble in aqueous media which contains covalently attached to it mercapto, or thiol groups, thiosulfate, thiophosphate, thiourea, thiocarboxylic acid, dithiocarboxylic acid, thiocarbonate O-ester, dithiocarbonate O-ester groups, or combination thereof.

Aspect 6. The method of Aspect 5, wherein the one or more neutralizing agents is sodium thiosulfate, 2-mercaptoethanol, 2-(methylamino)ethanethiol, 2-aminoethanethiol, 2-(dimethylamino)ethanethiol, 2-mercapto-N,N,N-trimethylethanaminium and salts thereof, thiocarboxylic acids and salts thereof, thioacetic acid and salts thereof, thiopropionic acid and salts thereof, thiooxalic acid and salts thereof, thiomalonic acid and salts thereof, thiosuccinic acid and salts thereof, thioglycolic acid and salts thereof, thiolactic acid and salts thereof, dithiocarboxylic acids and salts thereof, dithioacetic acid and salts thereof, 2-mercaptoacetic acids and its salts, 2-mercaptopropionic acid and its salts, ethyl 2-mercaptoacetate, 2-mercaptosuccinic acid and its salts and esters, 2-(methylsulfonyl)methanethiol, (ethyl sulfonyl)methanethiol, sulfonyldimethanethiol, 2,2,2-trifluoroethanethiol, 1H-imidazole-5-thiol, imidazolidine-2-thione, 1,3-dimethylimidazolidine-2-thione, pyridine-2-thiol, 4-thioxo-3,4-dihydropyrimidin-2(1H)-one, 2-thioxodihydropyrimidine-4,6(1H,5H)-dione, 2-mercaptobenzoic acid and salts thereof, 4-mercaptobenzoic acid and salts thereof, thiophenol, 2-, 3-, or 4-mercaptoanisole, 2-mercaptopropane-1,2-diol, 2,3-dimercaptopropanol, or 1,3-dimercapto-2-propanol, and combinations thereof.

Aspect 7. The method of Aspect 5, wherein the mercaptan or thiol of the neutralizing agent has a pKa of dissociation of its —SH group between 4 and 10, preferably between 5 and 9, and even more preferably between 6 and 8, or close to the pH of the treated media.

Aspect 8. The method of Aspect 5, in which the mercaptan or the thiol of the neutralizing agent has a —SH group which is directly connected to a double bond, or aromatic structure, or fully or partially sp2 hybridized carbon atom.

Aspect 9. The method of Aspect 5, in which the neutralizing agent comprises at least one electron-accepting group, such as sulfone group (—S(O2)—R), or sulfoxide group (—S(O₂)—R), or ester group (C(O)OR) or amide group (—C(O)NH₂, —C(O)NHR, —C(O)NR₂), where R is any alkyl or substituted alkyl group, which electron-accepting group is attached to the carbon atom to which the SH group is attached.

Aspect 10. The method according to any one of Aspects 1 to 9, wherein the neutralizing agent is covalently bonded, optionally through a linking group, to a solid support.

Aspect 11. The method according to any one of Aspects 1 to 10, in which the one or more neutralizing agents are in contact with the sample containing a residual amount of the compound with Structure I for a period from one minute to 48 hours, preferably from 20 min to 24 h and even more preferably from 60 min to 8 h, and at temperatures from 0 to 100° C., preferably from 10 to 60° C., and even more preferably from 20 to 40° C., and at pH from 1 to 14, preferably from 4 to 9 and even more preferably from 6 to 8, and at concentrations of up to 1 M, preferably up to 0.1 M, and even more preferably at concentration of up to 10 mM.

Aspect 12. The method according to any one of Aspects 1 to 11, in which the concentration of the residual compound with Structure I is reduced after treatment with the neutralizing agent by at least 2 logs, preferably by at least 3 logs, and more preferably by at least 4 logs, still more preferably by at least 5 logs, still more preferably by at least 6 logs, still more preferably by at least 7 logs, still more preferably by at least 8 logs, still more preferably by at least 9 logs, still more preferably by at least 10 logs.

Aspect 13. The method according to any one of Aspects 1 to 12, wherein, after contacting of the residual compound of Structure I with the neutralizing agent, the products of neutralization or degradation of the compound of Structure I and/or the excess of the neutralizing agent are partially or completely removed from the treated sample by its treatment with a solid phase agent which is insoluble in the treated media, and which solid phase agent may be porous, microporous macroporous or gel type, or may be non-porous high dispersity and high surface area solid, and may be shaped as beads or particles of different size, from 1 μm to 1 cm, and which solid phase agent chemically reacts with and covalently binds, or absorbs, or otherwise sequester the products of neutralization or degradation of the compound(s) of Structure I and/or the neutralizing agent, followed by removal of the solid phase agent, preferably by filtration or sedimentation or centrifugation, or alternatively, the treatment is done by filtering of the media or composition through a cartridge containing the solid phase agent, or by contact of the media or composition with the solid phase agent through a permeable or a semi-permeable membrane, and the treatment can be done a single time, two times or multiple times, or until the desired reduction of the compounds of neutralization or degradation of compounds with Structure I is achieved, and which treatment can be done with a single solid phase agent, or with two or more different solid phase agents, either subsequently, or in a mixture.

Aspect 14. A method of Aspect 13, in which the solid phase agent absorbs the products of neutralization or degradation of the compound of Structure I and/or the excess of the neutralizing agent.

Aspect 15. A method of Aspect 14, in which the solid phase agent is activated carbon, or a reversed-phase resin, or porous or microporous hydrophobic organic polymer, such as polystyrene resin, or divinyl benzene cross-linked polystyrene resin, or polyacrylate or polymetacrylate resin modified with hydrophobic organic groups, such as C₄-C₁₈ alkyl groups.

Aspect 16. A method of Aspect 15, in which the solid phase agent is a cationite or anionite and forms ion-pairs with the product of neutralization or decomposition of compound of Structure I and/or the excess of the neutralizing agent, when the neutralizing agent is anionic or cationic under the pH of treatment.

Aspect 17. A method of Aspect 16, in which the cationite is an organic polymer, preferably cross-linked and bearing anionic groups such as sulfo, or sulfonic, or carboxylic groups, which are ion-pairing form with cations, such as sodium, potassium, or ammonium or substituted ammonium cations or with hydrogen cation.

Aspect 18. A method of Aspect 16, in which the anionite is an organic polymer, preferably cross-linked and bearing cationic groups, such as protonated amino, or alkyl substituted amino groups such as mono-, di- or trimethyl amine groups, or quaternary ammonium groups, such as tetramethylammonium groups, which groups are in ion-pairing form with anions, such as chloride, sulfate, citrate, or hydroxyl anions.

Aspect 19. A method of Aspect 13, in which the solid phase agent is a polymer, preferably cross-linked, which have attached to it thiosulfate groups ion-paired with acceptable cations, such as sodium and having the formula P—R—S—SO₃⁻Na⁺, where P is the polymer, R is a covalent bond or any divalent linker, and which groups react with the excess of the mercapto, or thiol type of neutralizing agent of formula R¹SH or R¹SH or R¹S⁻Cat⁺, where Cat⁺ is an acceptable cation, such as sodium by an exchange reaction resulting in covalent binding of the inactivator to the polymer through a disulfide bond as per the following formula P—R—S—S—R¹ and release of thiosulfate anion, S₂O₃²⁻; or the said polymer have epoxy or substitute epoxy attached to it, either directly or through a linker, and which epoxy groups react with the excess of the mercapto, or thiol type of neutralizing agent of formula R¹SH or R¹S⁻Cat⁺, where Cat⁺ is an acceptable cation, such as sodium, opening the epoxy groups and covalently attaching the neutralizing agent to the said polymer.

Aspect 20. The method according to any one of Aspects 1 to 19, wherein the sample is a composition, utility, surface, device or organism.

Aspect 21. The method according to any one of Aspects 1 to 19, wherein the sample is blood or blood products, bodily fluids, medium originated from eukaryotes or prokaryotes, vaccine preparation compositions, biologics or biologic preparations, clinical sample, biopsy, research sample, cosmetics, pharmaceutical compositions, disposables, instrument, aquatic fluid conduits, pipes, hoses, heat exchanges, or aquatic vessels and their surfaces.

Aspect 22. The method according to any one of Aspects 1 to 19, wherein the sample is blood or a blood product.

Aspect 23. A method for inactivation, reduction or removal of pathogens or undesired organisms from a sample comprising:
treatment of the sample with compound with Structure I:

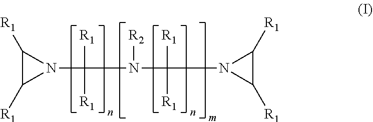

wherein:
R₁ is independently selected for each occurrence from H, Cl, F, an alkyl group, CH₃, CH₂CH₃, CH(CH₃)₂, an alkenyl group, a phenyl group, an alkyloxy group, an acyloxy group, or other substituted alkyl group,
R₂ is independently selected for each occurrence from H, CH₃, CH₂CH₃, CH(CH₃)₂, an alkyl group, an alkenyl group, a phenyl group, a cycloalkyl group, an alkyloxy group, or substituted alkyl, alkenyl, cycloalkyl or phenyl group, or moiety of Structure II:

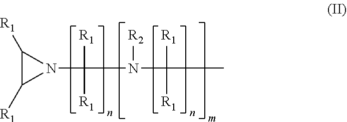

wherein;
n is independently for each occurrence 3, 4, or 5;
m is independently for each occurrence 1, 2, 3, 4, 5, 6, 7, 8, 9 or 10;
or a chemically acceptable salt, hydrate, or solvate thereof;
followed by incubation for sufficient time to allow for the desired effect of compound or compound with Structure I on the pathogens or undesired organisms to take place;
(ii) treatment of the sample with a solid phase agent which is not soluble in the treated media, and which solid phase agent may be porous, microporous macroporous or gel type, or may be a non-porous high dispersity and high surface area solid, and may be shaped as beads or particles of different size, such as from 1 μm to 1 cm, and which solid phase agent chemically reacts with and covalently binds, or absorbs, or otherwise sequesters the residual compound of Structure I or the product(s) of its degradation;
(iii) removal of the solid phase agent, preferably by filtration, sedimentation or centrifugation; or alternatively, the treatment is done by filtering of the sample through a cartridge containing the solid phase agent, or by contact of the sample with the solid phase agent trough a permeable or a semi-permeable membrane; and the said treatment can be done a single time, or two times or multiple time, or until the desired reduction of the compounds with Structure I or the products of its degradation is achieved, and which treatment can be done with a single solid phase agent, or with two or more different solid phase agents, either subsequently, or in a mixture.

Aspect 24. The method according to Aspect 23, wherein the compound of Structure I has the Structure IA:

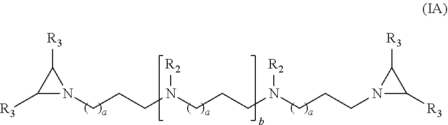

wherein:
each R₂ is independently selected for each occurrence from H, an alkyl group, CH₃, CH₂CH₃, CH(CH₃)₂, an alkenyl group, a phenyl group, a cycloalkyl group, an alkyloxy group, or substituted alkyl, alkenyl, cycloalkyl, phenyl group, or a moiety of Structure IIA:

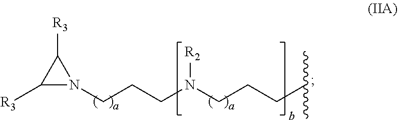

each R₃ is independently selected for each occurrence from H, Cl, F, an alkyl group, CH₃, CH₂CH₃, CH(CH₃)₂, an alkenyl group, a phenyl group, an alkyloxy group, an acyloxy group, or a substituted alkyl group;
each a is independently selected for each occurrence from 1, 2 or 3; and
each b is independently selected for each occurrence from 0, 1, 2, 3, 4, 5 or 6.

Aspect 25. The method according to Aspect 23, wherein the compound of Structure I has the Structure IB.

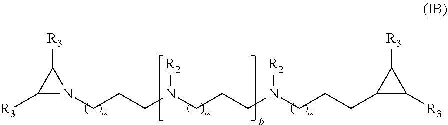

wherein
each R₂ is independently selected for each occurrence from H, CH₃, CH₂CH₃, or CH(CH₃)₂;
each R₃ is independently selected for each occurrence from H, CH₃, CH₂CH₃, or CH(CH₃)₂;
each a is independently selected for each occurrence from 1, 2 or 3; and
b is selected from 0, 1, 2, 3, 4, 5 or 6.

Aspect 26. A method according to any one of Aspects 23 to 25, in which the solid phase agent contains reactive groups, which chemically react with, and covalently bind the compound of Structure I.

Aspect 27. The method of Aspect 26, wherein the reactive groups, which can react with and open the aziridine rings of the compound of Structure I, are nucleophilic groups, such as thiosulfate, —OS(O)(O⁻)S⁻, or thiosufonate —S(O)(O⁻)S⁻, or mercapto or thiol groups, —SH, —CH₂SH, —CH₂CH₂SH, —CF₂CH₂SH, —OCH₂CH₂SH, —NH₂CH₂CH₂SH, —NH(Me)CH₂CH₂SH, —N(Me₂)CH₂CH₂SH, —COCH₂SH, —S(O₂)CH₂SH, -thiourea, —NHC(S)NH₂, or substituted thiourea groups, thiocarboxylic acid, —C(O)S⁻, dithiocarboxylic acid, —C(S)S⁻, thiocarbonate O-esters, —OC(O)S⁻, dithiocarbonate O-esters, or xanthates, —OC(S)S⁻, thiophosphonate, —PO(OH)SH, and thiophosphate, —OPO(OH)SH, o-, m-, or p-thiophenyl groups, —C₆H₄SH, thiosalicylate groups, m-, or p-thiobenzoate groups, —O₂CC₆H₄SH, or there salt forms.

Aspect 28. The method according to Aspect 27, in which the mercapto, or thiol or —SH group is directly connected to a double bond, or aromatic structure, or fully or partially sp² hybridized carbon atom.

Aspect 29. The method according to Aspect 27 or 28, in which the —SH groups have pKₐ of dissociation to —S⁻ and H⁺ of less than 10, preferably less than 9, and most preferably less than 8.

Aspect 30. The method according to any one of Aspects 23 to 29, wherein the solid phase agent is a porous, microporous, or a gel type of organic polymer.

Aspect 31. The method of Aspect 30, in which the organic polymer is a hydrophilic organic polymer, or polymer which is wettable, or can expand, or swell in aqueous based media.

Aspect 32. The method of Aspect 30 or 31, in which the organic polymer, preferably cross-linked, is a polystyrene polymer, or polyacrylate polymer, or polymethacrylate polymer, or polyurethane based polymer, or polyamide based polymer, or dextran based polymer, such as, but not limited to Sephadex®, or agarose based polymer, such as but not limited to Sepharose®, or a cellulose based polymer, or modified cellulose based polymer, such as but not limited to carboxymethylcellulose, or diethylaminoethyl cellulose, or methylcellulose, or other polysaccharide based polymer, or any other linear, branched, or cross-linked homo- or hetero-polymer or block copolymer, with iso- or atactic configuration, or with other tacticity, or may be any other appropriate macromolecule that is not soluble in the treated media.

Aspect 33. The method according to any one of Aspects 27 to 32, in which the nucleophilic groups can be one of different types and can be attached directly to the backbone of the polymer, or can be attached trough a divalent group, such as, but not limited to oxygen atom, sulfur atom, an —NH— group, methylene group, a mono- or disubstituted methylene group, ethylene, or substituted ethylene group, propylene or substituted propylene group, oxymethylene or oxyethylene group, or a di-, tri-, or polyvalent linker, such as, but not limited to oligo- or polyoxyethylene, oligo- or polyester, or polyamide type linker, which linker might be straight-chained or branched, or dendrimeric and may contain one or more than one or many nucleophilic groups attached to it.

Aspect 34. The method according to any one of Aspects 30 to 33, in which the polymer contains not only nucleophilic groups, but also groups which, without reacting with the compound of Structure I, assist its reaction with the nucleophilic groups by, but not limited to, enhancing the nucleophilicity of the nucleophilic group through the so called neighboring effect, or neighboring electron pair effect, or by enhancing of the deprotonation of the nucleophilic group, or by H-bonding to the nucleophilic group, or by interacting with, and lowering of the energy of the transition state formed between compound of Structure I and the nucleophilic group, or by non-covalent binding or ion-pairing with the compound of Structure I thus increasing their local concentration, or by protonating of the aziridine nitrogens of compound(s) of Structure I thus increasing their reactivity.

Aspect 35. The method according to any one of Aspects 30 to 34, in which the organic polymer has attached hydrophilic groups in sufficient number as to increase the polymer hydrophilicity or wettablility or improve the polymer properties, such as, but not limited to, inertness toward the components of the sample, or the composition, or organism, or biological fluids.

Aspect 36. The method of Aspect 35, in which the organic polymer is divinylbenzene cross-linked polystyrene and the polar groups are ethylene glycol oligomers, or polyethylene glycols with molecular mass from 150 to 100,000 Da, preferably from 2,000 to 40,000 Da, and even more preferably from 4,000 to 20,000 Da and with density of up to one group at every monomer unit, or sulfo groups (sulfonic acid groups, —SO₃⁻), or the polymer is acrylate or metacrylate polymer and the polar groups are polyols, such as, but not limited to 2-hydroxyethyl, 2,3-dihydroxypropyl, di-, tri-, tetra-, penta-, or oligo-, or polyethylene glycol, and the said polar groups are attached to the Cl, or the carbonyl group of the acrylate or metacrylate polymer in density sufficient to achieve the desired hydrophilicity or other advantageous properties, which might be, without being limited to, lack of immunogenicity, or lack of thrombogenicity, or lack of binding or affinity to proteins, or receptors, or other components of the treated sample or composition or bodily fluids.

Aspect 37. The method according to any one of Aspects 23 to 36, in which the solid phase agents forms multiple ion pairs with the positively charged nitrogen atoms of residual compound of Structure I.

Aspect 38. The method of Aspect 37, in which the solid phase agent is an organic polymer, micro-, or macroporous, or gel type organic polymer, preferably cross-linked and bearing anionic groups such as sulfo, or sulfonic, or carboxylic groups which are in ion-pairing form with cations, such as sodium, potassium, or ammonium or substituted ammonium cations or hydrogen cations.

Aspect 39. The method of Aspect 38, in which the polymer is a divinyl cross-linked polystyrene polymer, containing sulfonic groups in the sodium form and in density of up to 1.5 mili equivalents per gram polymer.

Aspect 40. The method of Aspect 38, in which the polymer is a diacrylate cross linked polyacrylate or methacrylate and the anionic groups are sulfonic or carboxylic groups in the sodium form and in density of up to 4 miliequivalent per gram of polymer.

Aspect 41. The method according to any one of Aspects 1 to 40, in which the pathogens or undesired organisms are: infections disease causing organisms, such as, but not limited to viruses, including enveloped and non-enveloped viruses, DNA or RNA viruses and bacteriophages, prions, prokaryote, bacteria, including Gram-positive or Gram-negative bacteria, spore forming bacteria or bacterial spores, mycoplasma, archaea, and bacterial films; eukaryote, single-, or multicellular eukaryote, including but not limited to, fungi, protozoa, single or multicellular parasite, helminths, schistosomes or nematodes or their eggs, single or multicellular algae and crustacean or any combination thereof including leaches, biofilms or biofouling systems.

Aspect 42. The method according to any one of Aspects 1 to 41, wherein the treated sample is selected from human or animal blood, leukodepleated blood, and blood products, including plasma, red blood cells, platelets, serum, or plasma components, factors or enzymes, transfusion blood and blood components intended for transfusion, apheresis blood components, bodily fluids, animal serum, including serum used as cell culture additive, medium originated from eukaryotes or prokaryotes, vaccine preparation compositions, cosmetic and pharmaceutical compositions; the utility can be any industrial or household equipment, appliances, apparatuses, mechanisms, machinery, or materials, or any other articles where pathogens, microorganisms, or other organisms presence might be undesirable or needs to be controlled; the surface can be the surface of utensils, devices or utilities, including pipe, duct, hose, pipeline, vent, heat exchanger, sewer, channel, or any other fluid or gas conduit, or any body's surface which is in contact with fluid, such as sea vessels, screens or filters where pathogens, microorganisms, or other organisms presence is undesirable or in need of control including biofouling; the organism can be an animal, mammal or human or parts thereof, including biological samples, preparations and biopsies.

Aspect 43. The method according to any one of Aspects 1 to 42, wherein the pathogen(s) or microorganism(s) are treated with a composition containing one or more compounds of Structure I, and where the composition can be formulated as a liquid, solution, gel, solid, powder, particles, or can be encapsulated, dissolved, dispersed, pulverized, micronized, or converted to nano-particles, or in other formulated forms or in combinations thereof.

Aspect 44. The method according to any one of Aspects 1 to 43, in which the sample or composition is treated with a compound with Structure I for a period of time from one minute to 48 hours, preferably from 20 min to 24 h and even more preferably from 60 min to 8 h, and at temperatures from 0 to 100° C., preferably from 10 to 60° C., and even more preferably from 20 to 40° C.; and at pH from 1 to 14, preferably from 4 to 9 and even more preferably from 6 to 8; and at concentrations from 10 nM to 10 mM, preferably from 1 μM to 1 mM, still more preferably from 100 μM to 500

Aspect 45. The method according to any one of Aspects 1 to 44, in which the titer of at least one of the pathogens or undesired organisms present in the treated sample is reduced by at least 50%, preferably by at least 1 log, more preferably by at least 2 logs, still more preferably by at least 3 logs, still more preferably by at least 4 logs, still more preferably by at least 5 logs, still more preferably by at least 6 logs, still more preferably by at least 7 logs, still more preferably by at least 8 logs, still more preferably by at least 9 logs, still more preferably by at least 10 logs or more.

Aspect 46. The method according any one of Aspects 1 to 45, in which the pathogen(s) or microorganism(s) are present in an organism, which organism may be an animal, a mammal or a human, and the treatment with compounds of Structure I or formulations of compounds of Structure I is done in vivo, by intravenous, oral, topical, rectal, subcutaneous, intramuscular administration, by inhalation, or by combination thereof, and the said treatment can be done by a single administration, by multiple administrations, or by continuous administration and at dose(s) sufficient to achieve the desired pathogen reduction.

Aspect 47. The method of Aspect 46, in which the removal, or neutralization, or inactivation of the compounds of Structure I and, optionally, the removal of the products of neutralization of the compounds of Structure I and/or the excess of the neutralizing agents is done by ex-vivo treatment of the bodily fluids of the organism, which bodily fluids are returned or transfused back to the organism.

Aspect 48. The method according to any one of Aspects 1 to 47, in which the pathogen(s) or microorganism(s) are present in an animal or human and the treatment with compound of Structure I, and the removal or neutralization of the compound of Structure I and, optionally, the products of their neutralization or degradation and/or the excess of the neutralizing agents is done ex-vivo by treatment of the bodily fluids of the animal or human, such as blood or plasma, which might be collected by apheresis and which fluids after treatment are returned or transfused back to the animal or human.

Aspect 49. A method according to any one of Aspects 1 and 48, in which at least one of the pathogens or undesired organisms is resistant to one or more anti pathogen treatments, or may not be susceptible to any treatment except to treatment by compounds with Structure I.

Aspect 50. The method according to any one of Aspects 1 to 49, in which the compound with Structure I is in a salt form with an organic or inorganic anion, preferably an anion of low nucleophilicity, such as sulfate, perchlorate, methansulfonate or tetrafluoroborate, or in the form of solid solution with a solid of good aqueous solubility and melting point below above 40 and below 120° C., such as, but not limited to polyethylene glycol with different molecular weights.

We claim:

1. A method for inactivation or reduction of pathogens or undesired organisms from a sample, comprising:
(i) treatment of the sample, with a compound having Structure I: 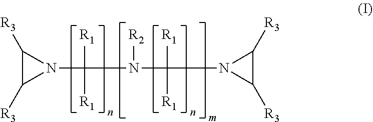
wherein:
each R₁ is independently selected for each occurrence from H, CH₃, CH₂CH₃, CH(CH₃)₂, Cl, F, an alkyl group, an alkenyl group, a phenyl group, an alkyloxy group, an acyloxy group, or substituted alkyl group,
each R2 is independently selected for each occurrence from H, CH₃, CH₂CH₃, CH(CH₃)₂, an alkyl group, an alkenyl group, a phenyl group, a cycloalkyl group, an alkyloxy group, or substituted alkyl, substituted alkenyl, substituted cycloalkyl or substituted phenyl group, or a moiety of Structure II: 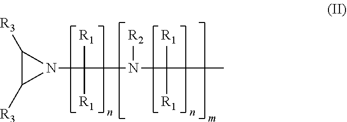
each R3 is independently selected for each occurrence from H, CH₃, CH₂CH₃, CH(CH₃)₂, Cl, F, an alkyl group, an alkenyl group, a phenyl group, an alkyloxy group, an acyloxy group, or other substituted alkyl group;
each n is independently for each occurrence 3, 4, or 5;
each m is independently for each occurrence 1, 2, 3, 4, 5, 6, 7, 8, 9 or 10; or a chemically acceptable salt, hydrate, or solvate thereof;
(ii) incubation for sufficient time for inactivation or reduction of pathogens or undesired organisms from the sample;
(iii) treatment of the sample (a) with one or more neutralizing agents which eliminate or reduce the toxicity or other undesirable properties of the compound with Structure I, or (b) with one or more solid phase agents which absorbs, or covalently binds the compounds with Structure I.

2. The method according to claim 1, wherein the compound of Structure I has the Structure IA: 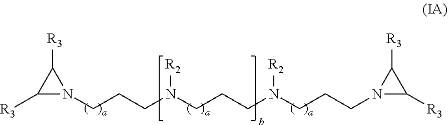
wherein:
each R₂ is independently selected for each occurrence from H, an alkyl group, CH₃, CH₂CH₃, CH(CH₃)₂, an alkenyl group, a phenyl group, a cycloalkyl group, an alkyloxy group, or substituted alkyl, alkenyl, cycloalkyl, phenyl group, or a moiety of Structure IIA: 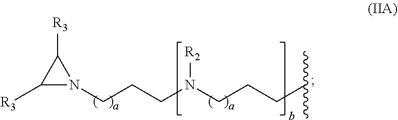
each R₃ is independently selected for each occurrence from H, Cl, F, an alkyl group, CH₃, CH₂CH₃, CH(CH₃)₂, an alkenyl group, a phenyl group, an alkyloxy group, an acyloxy group, or a substituted alkyl group;
each a is independently selected for each occurrence from 1, 2 or 3; and
each b is independently selected for each occurrence from 0, 1, 2, 3, 4, 5 or 6.

3. The method according to claim 1, wherein the compound of Structure I has the Structure IB: 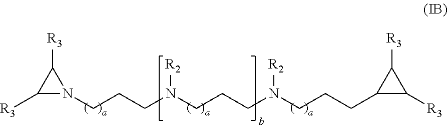
wherein:
each R2 is independently selected for each occurrence from H, CH₃, CH₂CH₃, or CH(CH₃)₂;
each R3 is independently selected for each occurrence from H, CH₃, CH₂CH₃, or CH(CH₃)₂;
each a is independently selected for each occurrence from 1, 2 or 3; and
b is selected from 0, 1, 2, 3, 4, 5 or 6.

4. The method according claim 1, where the one or more neutralizing agents are nucleophilic compounds which eliminate the alkylating properties of the compound of Structure I by reacting with and opening of the aziridine rings of the compound of Structure I.

5. The method of claim 4, wherein the one or more neutralizing agents are selected from the group consisting of thiosulfates, thiophosphates, thiourea or substituted thioureas, thiocarboxylic acids and salts thereof, dithiocarboxylic acid and salts thereof, thiocarbonate salt, dithiocarbonate salt, salt of thiocarbonate O-esters, salt of dithiocarbonate O-esters, mercaptans or thiols, or their salts, or substituted mercaptans, or substituted thiols, or polymercaptan or polythiols and their salts, or any combination thereof, or organic polymer soluble in aqueous media which contains covalently attached to it mercapto, or thiol groups, thiosulfate, thiophosphate, thiourea, thiocarboxylic acid, dithiocarboxylic acid, thiocarbonate O-ester, dithiocarbonate O-ester, or a combination thereof.

6. The method of claim 5, wherein the one or more neutralizing agents is selected from the group consisting of sodium thiosulfate, 2-mercaptoethanol, 2-(methylamino)ethanethiol, 2-aminoethanethiol, 2-(dimethylamino)ethanethiol, 2-mercapto-N,N,N-trimethylethanaminium and salts thereof, thiocarboxylic acids and salts thereof, thioacetic acid and salts thereof, thiopropionic acid and salts thereof, thiooxalic acid and salts thereof, thiomalonic acid and salts thereof, thiosuccinic acid and salts thereof, thioglycolic acid and salts thereof, thiolactic acid and salts thereof, dithiocarboxylic acids and salts thereof, dithioacetic acid and salts thereof, 2-mercaptoacetic acids and its salts, 2-mercaptopropionic acid and its salts, ethyl 2-mercaptoacetate, 2-mercaptosuccinic acid and its salts and esters, 2-(methylsulfonyl)methanethiol, (ethyl sulfonyl)methanethiol, sulfonyldimethanethiol, 2,2,2-trifluoroethanethiol, 1H-imidazole-5-thiol, imidazolidine-2-thione, 1,3-dimethylimidazolidine-2-thione, pyridine-2-thiol, 4-thioxo-3,4-dihydropyrimidin-2(1H)-one, 2-thioxodihydropyrimidine-4,6(1H,5H)-dione, 2-mercaptobenzoic acid and salts thereof, 4-mercaptobenzoic acid and salts thereof, thiophenol, 2-, 3-, or 4-mercaptoanisole, 2-mercaptopropane-1,2-diol, 2,3-dimercaptopropanol, or 1,3-dimercapto-2-propanol, and combinations thereof.

7. The method according to claim 1, wherein the neutralizing agent is covalently bound to a solid phase support.

8. The method according to claim 7, wherein the solid phase support is a porous, microporous, or a gel type of organic polymer.

9. The method of claim 8, in which the organic polymer is a hydrophilic organic polymer, or polymer which is wettable, or can expand, or swell in aqueous based media.

10. The method of claim 8, in which the organic polymer is selected from the group consisting of a polystyrene polymer, polyacrylate polymer, polymethacrylate polymer, polyurethane based polymer, polyamide based polymer, dextran based polymer, agarose based polymer, a cellulose based polymer, or a modified cellulose based polymer, diethylaminoethyl cellulose, methylcellulose, and polysaccharide based polymer.

11. The method according to claim 1, in which the nucleophilic groups of the neutralizing agent is attached directly to the backbone of the polymer, or is attached trough a divalent group, selected from an oxygen atom, sulfur atom, an —NH— group, methylene group, a mono- or disubstituted methylene group, ethylene, or substituted ethylene group, propylene or substituted propylene group, oxymethylene or oxyethylene group, or a di-, tri-, or polyvalent linker selected from an oligo- or polyoxyethylene, oligo- or polyester, or polyamide type linker, which linker is straight-chained or branched, or dendrimeric and contains one or more nucleophilic groups attached to it.

12. The method according to claim 1, wherein, after contacting of the residual compound of Structure I with the neutralizing agent, the products of neutralization or degradation of the compound of Structure I and/or the excess of the neutralizing agent are reduced or removed from the treated sample by its treatment with a solid phase agent which is insoluble in the treated media, which solid phase agent chemically reacts with and covalently binds, or absorbs the products of neutralization or degradation of the compound of Structure I and/or the neutralizing agent, followed by removal of the treated sample from the solid phase agent.

13. The method of claim 12, in which the solid phase agent absorbs the products of neutralization or degradation of the compound of Structure I and/or the excess of the neutralizing agent.

14. The method of claim 13, in which the solid phase agent is selected from the group consisting of activated carbon, reversed phase resin, porous or microporous hydrophobic organic polymer, divinyl benzene cross-linked polystyrene resin, polyacrylate or polymetacrylate resin modified with hydrophobic organic groups.

15. The method according to claim 1, in which the one or more neutralizing agents are in contact with the sample containing a residual amount of the compound with Structure I for a period from one minute to 48 hours, and at temperatures from 0 to 100° C., and at pH from 1 to 14, and at concentrations of up to 1 M.

16. The method according to claim 1, in which the concentration of the residual compound with Structure I is reduced after treatment with the neutralizing agent by at least 2 logs.

17. The method according to claim 1, in which the pathogens or undesired organisms are one or more of: infections disease causing organisms, viruses, enveloped and non-enveloped viruses, DNA or RNA viruses and bacteriophages, prions, prokaryote, bacteria, Gram-positive or Gram-negative bacteria, spore forming bacteria or bacterial spores, mycoplasma, archaea, and bacterial films; eukaryote, single-, or multicellular eukaryote, fungi, protozoa, single or multicellular parasite, helminths, schistosomes or nematodes or their eggs, single or multicellular algae and crustacean or biofilms or biofouling systems, or any combination thereof.

18. The method according to claim 1, wherein the sample is a composition, surface, device or organism.

19. The method according to claim 1, wherein the sample is blood or blood products, bodily fluids, medium originated from eukaryotes or prokaryotes, vaccine preparation compositions, biologics or biologic preparations, clinical sample, biopsy, research sample, cosmetics, pharmaceutical compositions, disposables, instrument, aquatic fluid conduits, pipes, hoses, heat exchanges, or aquatic vessels and their surfaces.

20. The method according to claim 1, wherein the sample is blood or a blood product.
